(19) United States
(12) Patent Application Publication
(10) Pub. No.: US 2022/0105170 A1
(43) Pub. Date: Apr. 7, 2022

(54) AFRICAN SWINE FEVER VACCINE

(71) Applicants: Phibro Animal Health Corporation, Teaneck, NJ (US); Life Science Research Israel Ltd., Ness-Ziona (IL)

(72) Inventors: Avner Finger, Pardes Hanna-Karkur (IL); Avi Zrachya, Airport City (IL); Ofer Cohen, Ness-Ziona (IL); Anat Zvi, Ness-Ziona (IL)

(73) Assignees: Phibro Animal Health Corporation, Teaneck, NJ (US); Life Science Research Israel Ltd., Ness-Ziona (IL)

(21) Appl. No.: 17/554,232

(22) Filed: Dec. 17, 2021

Related U.S. Application Data
(63) Continuation of application No. PCT/US2020/039846, filed on Jun. 26, 2020.
(60) Provisional application No. 62/941,381, filed on Nov. 27, 2019.
(60) Provisional application No. 62/868,483, filed on Jun. 28, 2019.

(51) Int. Cl. A61K 39/12; A61K 39/39; A61P 31/20; C12N 7/00

(52) U.S. Cl. CPC A61K 39/12; A61K 39/39; A61K 2039/55588; C12N 7/00; A61P 31/20

(57) ABSTRACT

Peptides predicted to be immunogenic against African swine fever virus (ASFV) and vaccine compositions that include the peptides are disclosed herein. In some embodiments, these compositions comprise or consist of one or more peptides comprising the amino acid sequence set forth in SEQ ID NOs: 2-2273. In other embodiments, the compositions comprise viral vectors or host cells, or combinations thereof, that comprise one or more of the peptides. In other embodiments, the compositions comprise nucleic acid molecules comprising one or more of the peptides. The compositions disclosed can include one or more additional components, such as, but not limited to, a carrier, an adjuvant, an additional therapeutic, or combinations thereof. Containers and kits that comprise the compositions are described. Uses of the compositions can include administration to an animal to induce an immune response in the animal, or to immunize the animal against ASFV. Administration can be accomplished using one or more of various methods as described herein, such as intramuscular or intranasal administration.

### CROSS REFERENCE TO RELATED APPLICATIONS

This application is a continuation of International Application No. PCT/US2020/039846, filed on Jun. 26, 2020, which was published in English under PCT Article 21(2), which in turn claims the benefit under 35 U.S.C. § 119(e) of the earlier filing dates of U.S. Provisional Applications, Nos. 62/868,483, filed on Jun. 28, 2019, and 62/941,381, filed on Nov. 27, 2019. The prior applications are incorporated herein by reference in their entireties.

### FIELD

This disclosure concerns embodiments of a composition comprising a peptide or mixture of peptides associated with the African swine fever virus (ASFV), or comprising one or more vectors comprising one or more such peptides, and embodiments of a method for administering such a composition or compositions to elicit an immune response against ASFV, and/or to mitigate or inhibit symptoms associated with viral infections.

### PARTIES TO JOINT RESEARCH AGREEMENT

Phibro Animal Health Holdings, Inc. and Life Science Research Israel Ltd. executed a Joint Research Agreement on or before the date subject matter disclosed and claimed by the present application was made, and such subject matter was made as a result of activities undertaken within the scope of the Joint Research Agreement.

### BACKGROUND

African swine fever (ASF), caused by African swine fever virus (ASFV), is one of the most serious viral diseases affecting domestic pigs, in part due to high infectivity and mortality rates. ASFV infection usually results in acute hemorrhagic disease with a mortality rate approaching 100% in domestic swine. The virus can be transmitted by ingestion, contact, or through ticks of the genus Ornithodoros.

ASFV was first identified in Kenya in the 1920s, and is endemic in Africa, where wild pig species act as reservoirs for the virus. In the 1950s, ASFV spread throughout Europe, including Spain, Portugal, Italy, and France, but was eradicated from these countries, except for the island of Sardinia, Italy, by the mid-1990s. However, the disease was introduced into Georgia in 2007, and then spread throughout Eastern Europe and Russia. The virus continued to spread worldwide and has now been reported in 37 countries or regions. In 2018, at least four countries, including Hungary, Bulgaria, Belgium, and China, reported their first ever ASFV outbreaks to the World Organization for Animal Health (OIE; http://www.oie.int/).

The first ASF case in China was reported on Aug. 3, 2018. By Jan. 19, 2019, at least 100 ASF cases had occurred in 23 provinces or regions across the country (http://www.oie.int/). ASF continues to spread throughout China, severely threatening the country's domestic swine population, which accounts for more than 50% of the swine population globally. ASFV is the only member of the Asfarviridae family and has a linear, double-stranded DNA genome. ASF is currently diagnosed in China by detecting viral genes using real-time PCR and partial genome sequence analysis. There is currently no effective vaccine to prevent ASF and the disease therefore poses a major threat to both the swine industry and global food security. SUMMARY

Certain embodiments of the present disclosure concern an immunogenic peptide or peptides associated with ASFV, and compositions comprising one or more such peptides selected from SEQ ID NOs. 2-2273. In particular embodiments, the peptides are expressed by the ASFV strain, China/2018/AnhuiXCGQ. A composition may comprise a nucleic acid molecule, host cell, and/or vector, such as a viral or bacterial vector, encoding one or more peptides selected from SEQ ID NOs. 2-2273.

Some embodiments of the present disclosure concern one or more immunogenic peptides of SEQ ID NOs. 2-2273, one or more constructs (for example, one or more amino acid sequences of SEQ ID NOs. 2310-2330), one or more domains (also referred to herein as “hotspots” as described in Example 3; for example, one or more amino acid sequences of SEQ ID NOs: 2331-2335), and/or one or more full- and/or partial-length ASFV proteins (for example, one or more proteins of SEQ ID NOs: 2323-2329 and/or nucleic acids of SEQ ID NOs. 2339-2345). A composition may comprise one or more vectors and/or cells and/or nucleic acid molecules comprising or encoding one or more of the peptides, constructs, domains, and/or full- and/or partial-length ASFV proteins.

Embodiments of a method for using disclosed peptides, constructs, compositions, isolated nucleic acids, vectors, and/or host cells are also provided. For example, one or more peptides, compositions, isolated nucleic acids, vectors, and/or host cells, may be administered, such as by oral, intramuscular, topical, and/or mucosal administration, to an animal, such as an ungulate, and even more particularly a swine, to stimulate an immune response, induce immunity in the animal, and/or reduce or ameliorate at least one symptom associated with a viral infection, such as viral infection associated with ASF. Such method can be used to treat or prophylactically vaccinate adult and/or juvenile animals. In some embodiments, a composition may include a pharmaceutically acceptable carrier, an adjuvant, an additional therapeutic, or a combination thereof. Additional therapeutics may include compounds or compositions that reduce or alleviate the symptoms of ASF, or other compositions, such as vaccines against other infections common in swine, particularly infections or conditions that may be exacerbated by ASF.

Certain embodiments comprise one or more peptides of SEQ ID NOs. 2-2273 wherein one or more amino acids of a peptide is substituted with another one or more amino acids, or wherein an amino acid in the peptide is inserted or deleted, or combinations thereof, provided that the resultant peptide or peptides are capable of inducing an immune response and/or ameliorating one or more symptoms associated with ASFV. A peptide may be produced by any suitable technique, including chemical synthesis and/or intracellular synthesis using recombinant techniques. Some embodiments comprise one or more peptides from 5 to at least 50 amino acids in length, such as, for example, 6-40, 8-30, 10-20, or 8-11 amino acids in length. A disclosed immunogenic peptide or peptides may be modified, for example, for the purpose of stabilizing peptide conformation, improving peptide stability against enzymatic degradation, improving peptide stability in vivo, or combinations thereof. Such modifications can include, for example, glycosylation, PEGylation, lipidation, cyclisation, acetylation, amidation, conjugation, D-amino acid incorporation, a similar modification, or combinations thereof.

Some disclosed embodiments concern one or more isolated nucleic acid molecules that encode the amino acid sequence of one or more peptides of SEQ ID NOs 2-2273, or that result from the substitution of some or any of the nucleotides of one or more of the nucleic acid molecules with other nucleotides, or from the insertion or deletion of one or more of such nucleotides, provided that the resultant peptides are capable of inducing an immune response and/or ameliorating one or more symptoms associated with ASF. Some embodiments concern a composition comprising one or more nucleic acid molecules that encode at least one peptide of SEQ ID NOs. 2-2273. A nucleic acid molecule encoding one or more peptides of SEQ ID NOs. 2-2273 may also encode additional components, such as, for example, expression control sequences, selection-related sequences, multiple cloning sites, similar sequences, or combinations thereof.

The peptides disclosed herein can be, and were, identified using various bioinformatics approaches, such as, for example, predictive algorithms that can identify high density clusters of putative immunogenic peptides and/or can identify potentially immunogenic peptides based on predicted WIC binding affinity. Immunogenicity of the disclosed peptides can be validated using various methods for measuring an immune response in vitro or in vivo, including, for example, ELISA and/or ELISpot assays, and/or observing symptom development in a challenged swine following vaccination. Such methods are known to those of ordinary skill in the art, and the present invention is not limited to using specific assays.

Multiple types and versions of vectors, nucleic acid molecules, and host cells encoding and/or expressing one or more peptides of SEQ ID NOs. 2-2273, one or more constructs (for example, one or more amino acid sequences of SEQ ID NOs. 2310-2330), one or more domains (also referred to herein as “hotspots” as described in Example 3; for example, one or more amino acid sequences of SEQ ID NOs: 2331-2335), and/or one or more full- and/or partial-length ASFV proteins (for example, one or more proteins of SEQ ID NOs: 2323-2329 and/or nucleic acids of SEQ ID NOs. 2339-2345). In some embodiments, one or more nucleic acid molecules encoding the one or more peptides, constructs, domains, and/or full- or partial-length ASFV proteins are incorporated into a viral vector, a host cell, and/or a larger nucleic acid construct, such as a plasmid, for administration to an animal. Methods of producing the vectors, nucleic acid molecules, and host cells are known to those of ordinary skill in the art, and the disclosure is not limited to using specific vector, nucleic acid molecule, or host cell production methods, or to specific vectors, nucleic acid molecules, or cell types.

Compositions comprising one or more vectors, and/or host cells, and/or nucleic acid molecules comprising one or more disclosed peptides, constructs, domains, and/or full- or partial-length ASFV proteins, for administration to an animal, such as mammals, including ungulates, and in particular embodiments to swine, also are disclosed. In some embodiments, one or more of the compositions may be used to elicit an immune response against ASFV and/or to immunize a subject against ASFV. A composition can be in a liquid solution or suspension, such as in PBS, water, an organic solvent or suspension aid, or another acceptable carrier. A composition can be in a dried, tablet, or powdered form, such as lyophilized or freeze dried, for direct administration to an animal, or alternatively can be reconstituted, for example with PBS, water, an organic solvent, or another acceptable carrier. A composition can also be in a gel or syrup form.

Disclosed immunogenic compositions may include other agents. Some embodiments concern a pharmaceutical composition comprising a therapeutically effective amount of a DNA construct encoding one or more disclosed peptides, constructs, domains, and/or full- or partial-length ASFV proteins, or of a vector encoding one or more of the peptides, constructs, domains, and/or full- or partial-length ASFV proteins, or of a cell comprising one or more of the peptides, together with one or more additional components. Additional components may include, but are not limited to, one or more adjuvants, carriers, and/or other therapeutics, such as, for example, other vaccines and/or compounds or compositions that reduce or alleviate the symptoms of ASF or conditions or infections that are exacerbated by ASF.

A composition may include two or more peptides of SEQ ID NOs. 2-2273 that are combined by polymerization to form an immunogenic polymer using one or more chemical methods, recombinant techniques, and/or enzymatic reactions. The peptides in the immunogenic polymer according to SEQ ID NOs. 2-2273 may be directly adjacent, or maybe separated by other sequences. A composition may include two or more disclosed peptides, constructs, domains, and/or full- or partial-length ASFV proteins that are combined by polymerization to form an immunogenic polymer using one or more chemical methods, recombinant techniques, and/or enzymatic reactions. The peptides, constructs, domains, and/or full- or partial-length ASFV proteins in the immunogenic polymer may be directly adjacent, or maybe separated by other sequences.

Also provided are cinnamon-derived compositions comprising a cinnamon extract, one or more fractions of a cinnamon extract, and/or one or more precipitates of a cinnamon extract. Certain embodiments concern an aqueous extract of cinnamon bark (Cinnamomum sp.), but other polar solvents may also be used. Useful extraction compositions may be made by any suitable process. Certain embodiments concern formation of an aqueous solution, which may then be centrifuged and a supernatant collected that includes an antiviral active fraction. A precipitate from the solution may also be formed, such as by evaporation or by adding a precipitation aid, such as, for example, a salt, such as a chloride salt.

Certain embodiments concern a pharmaceutical composition or a nutraceutical composition for the treatment of an infection comprising an effective amount of a cinnamon extract, one or more fractions of a cinnamon extract, and/or one or more precipitates of a cinnamon extract, together with a carrier suitable for pharmaceutical or nutraceutical compositions. Such compositions can also include one or more of the peptides, vectors, host cells, and/or nucleic acid molecules comprising one or more immunogenic peptides, constructs, domains, and/or full- or partial-length ASFV proteins disclosed herein. Such compositions can also include other components, such as at least one additional therapeutic or nutraceutic component. The compounds and/or compositions so formed have antiviral activity and can be administered by any suitable method as will be understood by a person of ordinary skill in the art, such as orally, nasally, parenterally, subcutaneously, and/or intramuscularly.

Also provided are embodiments of a method of treating a subject, such as an animal, particularly swine, that may have or be at risk of having ASF, with one or more disclosed peptides, constructs, domains, and/or full- or partial-length ASFV proteins, and/or one or more nucleic acids, vectors, host cells, or compositions comprising the one or more peptides, constructs, domains, and/or full- or partial-length ASFV proteins, or combinations thereof, as disclosed herein. An animal may be administered such compositions by one or more methods known to a person of ordinary skill in the art. Exemplary administration methods include, but are not limited to, topical, oral, subcutaneous, transdermal, intrathecal, intramuscular, intravenous, intraperitoneal, and similar administration routes, or combinations thereof. In certain embodiments, compositions may be administered as a single dose or as multiple doses (for example, boosters). Different administrations can include one or more different compositions, combinations of compositions, or amounts thereof. For example, the second administration can be with the same, or with a different composition than, the first composition administered.

The dose administered to a subject should be sufficient to induce a beneficial therapeutic response in a subject over time, or to inhibit ASFV infection. The beneficial therapeutic response may require one or more doses, such as 1, 2, 3, 4, 5, 6, 7, 8, 9, or 10 doses, and more typically 2-4 doses, administered at the same or different times. In some embodiments, one or more compositions comprising the peptide(s), vectors, nucleic acid molecules, or host cells described herein, or combinations thereof, can be administered to an animal to produce an immune response against ASFV, and/or to immunize an animal against ASFV. The dose may vary from subject to subject or may be the same. An appropriate dose can be determined by one of ordinary skill in the art using routine experimentation.

Also provided are embodiments of a method for administering one or more disclosed peptides, constructs, domains, and/or full- or partial-length ASFV proteins, or one or more nucleic acids, vectors, host cells, or compositions comprising the one or more peptides, constructs, domains, and/or full- or partial-length ASFV proteins, or combinations thereof, to an animal to elicit or stimulate an immune response in the animal. In one embodiment, the method includes vaccinating or immunizing an animal against ASFV using a composition comprising a viral vector expressing one or more disclosed peptides, constructs, domains, and/or full- or partial-length ASFV proteins. In other embodiments, an animal is administered one or more compositions comprising a viral vector expressing one or more disclosed peptides, constructs, domains, and/or full- or partial-length ASFV proteins, and is subsequently administered a vaccine comprising a live attenuated ASFV. Methods of determining whether an immune response has been elicited or stimulated are known to those of ordinary skilled in the art. In some embodiments, an immune response is achieved if there is an observed reduction in illness (such as reduction or amelioration of symptoms), reduction in viral titers, reduction in mortality rate, or a combination thereof.

Certain disclosed embodiments concern a neutralized virus composition, particularly a neutralized AFSV virus, wherein the virus is neutralized by contact with a cinnamon extract. The neutralized virus composition can be used to vaccinate a subject. For example, the method may comprise providing a cinnamon-extract-neutralized AFSV virus composition, and vaccinating a subject with the composition. The subject may be a mammal, such as an ungulate, and even more particularly may be swine.

Also provided are containers that comprise one or more of the disclosed peptides, constructs, domains, and/or full- or partial-length ASFV proteins, or one or more nucleic acids, vectors, host cells, or compositions comprising or encoding the one or more peptides, constructs, domains, and/or full- or partial-length ASFV proteins, or combinations thereof. A container may be reusable or disposable. Also provided are kits that include one or more such containers. The one or more containers in the kit can include one or more additional components. In some examples, the kits also include a device or devices that permit administration of one or more of the compositions, or of one or more of the additional components, or combinations thereof, to an animal. The foregoing and other objects, features, and advantages of the invention will become

more apparent from the following detailed description, which proceeds with reference to the accompanying figures.

### BRIEF DESCRIPTION OF THE DRAWINGS

FIG. 1 The complete genome of the ASFV China/2018/AnhuiXCGQ strain (GenBank Accession No. MK128995.1) was screened for CD8+epitopes in relation to the known SLA class I alleles of the Yorkshire, Landrace, and Duroc swine breed lines. Candidate peptides were evaluated according to four criteria: (1) predicted binding affinity of the peptide to SLA class I molecules; (2) position in highly dense clusters of putative epitopes as a method to enrich positive responders; (3) coverage of SLA alleles and prioritization of highly prevalent alleles; and (4) the nature of the source protein (giving precedence to immunogens). Out of 212,394 putative peptides, 2,272 were selected for further evaluation. ELISpot assays were used to further screen the 2,272 peptides.

FIG. 2 Provides Elispot results—Positive Pool Separation—concerns pools of peptides (approximately 8-9 peptides per pool) that were screened using ELISpot assays conducted using lymphocytes from 8 swine, denoted 2S, 3S, 5S, 7S, 10S, 14S, 6H, 7H. Thirty-three pools out of a total of 238 “positive” pools (those pools for which the number of spots met or exceeded a threshold) were selected. The 33 positive pools contained 267 peptides, to which 9 individual peptides identified as positive in the full screen were added (for a total of 276 peptides), for further testing.

FIG. 3 Provides Elispot results—Positive Pool Separation—concerning 276 peptides identified in the pool screen (FIG. 2) that were assessed individually using ELISpot assays. Concanavalin A (ConA) was used as a positive control, and a negative control (medium only) was used to calculate permissive and strict thresholds (wherein “average of medium” denotes the average number of spots in wells with medium only, calculated for each swine plate separately, and “STDEV_P” denotes standard deviation based on the entire population). Of the 276 peptides tested, 201 met or exceeded the permissive threshold calculated for these ELISpot assays (Appendix IV), and of the 201 peptides, 125 met or exceeded the stringent threshold (Appendix VIII). Of the 125 peptides that met or exceeded the stringent threshold, 77 were identified for

which at least 20 spots were counted (Appendix V). FIG. 4 The 77 peptides described in FIG. 3 were mapped to their locations within ASFV proteins (Appendices V-VI). Forty-four of the 77 peptides clustered within seven ASFV proteins (Appendix VII). The peptides of SEQ ID NOs: 619, 621, 633, 636, 639, 640, 645, 651, 652, 653, and 662 mapped to ASFV protein A238L, an IκB-like protein (GenBank Accession No. AYW34011.1).

FIG. 5 The peptides of SEQ ID NOs: 496, 497, 527, 529, 541, and 544 mapped to ASFV protein A224L (IAP-like protein p27; GenBank Accession No. AYW34004.1) (Appendix VII).

FIG. 6 The peptides of SEQ ID NOs: 377, 400, 404, 435, 447, 449, 455, 456, 457, 461, 462, 463, and 467 mapped to ASFV protein MGF_505-7R (GenBank Accession No. AYW34001.1) (Appendix VII).

FIG. 7 The peptides of SEQ ID NOs: 553, 554, 561, 578, 584, and 589 mapped to ASFV protein MGF_360-15R (GenBank Accession No. AYW34010.1) (Appendix VII).

FIG. 8 The peptides of SEQ ID NOs: 1248, 1253, and 1280 mapped to ASFV zinc finger protein B385R (GenBank Accession No. AYW34052.1) (Appendix VII).

FIG. 9 The peptides of SEQ ID NOs: 468, 469, and 478 mapped to ASFV protein MGF_505-9R (GenBank Accession No. AYW34002.1) (Appendix VII).

FIG. 10 The peptides of SEQ ID NOs: 67 and 69 mapped to ASFV protein MGF_110-3L (GenBank Accession No. AYW33963.1) (Appendix VII).

FIGS. 11-34 show Coomassie blue-stained gel and western blotting results for each of 54 constructs expressed in E. coli at either 22° C. (FIG. 11-21) or 37° C. (FIGS. 22-34), along with the expected molecular weight and specific one or more tags for each construct. Sequences of constructs labeled 1-54 are provided in SEQ ID NOs. 2310-2330. While each construct included a His-tag for detection purposes, certain constructs also included at least one additional fusion protein, such as HLT, Sumo, or MBP. In FIGS. 11, 14, 17, 18, 21, 22, 25, 27, 30, and 33, if only “His” is shown in column three of the table, the construct included a His-tag, but no fusion protein (constructs shown as including a fusion protein also included a His-tag). Proteins were collected and then separated using polyacrylamide gel electrophoresis. As depicted in the Coomassie blue-stained gels and western blots, “M” shows the marker lane denoting band molecular weights, “S” represents proteins collected from cell culture supernatants, and “P” represents proteins collected from cell pellets.

FIG. 11 shows a table that provides the expected molecular weight (in kDa) (column 2) and the tag and/or fusion protein (column 3) associated with each construct of column 1. Expression analysis results for constructs listed in FIG. 11 (constructs 1-14) are shown in the Coomassie blue-stained gel of FIG. 12 and the western blot of FIG. 13.

FIG. 12 provides an image of a Coomassie blue-stained gel showing proteins collected from the cell pellet (P) or supernatant (S) of E. coli cultures grown at 22° C. E. coli cultures each expressed one of constructs 1-14 (FIG. 11).

FIG. 13 shows an image of a western blot that corresponds to the Coomassie blue-stained gel of FIG. 12. Relative expression levels of constructs 1-14 (FIG. 11) are shown, as detected using anti-His antibodies.

FIG. 14 shows a table that provides the expected molecular weight (in kDa) (column 2) and the tag and/or fusion protein (column 3) associated with each construct of column 1. Expression analysis results for constructs listed in FIG. 14 (constructs 15-28) are shown in the Coomassie blue-stained gel of FIG. 15 and the western blot of FIG. 16.

FIG. 15 provides an image of a Coomassie blue-stained gel showing proteins collected from the cell pellet (P) or supernatant (S) of E. coli cultures grown at 22° C. E. coli cultures each expressed one of constructs 15-28 (FIG. 14).

FIG. 16 shows an image of a western blot that corresponds to the Coomassie blue-stained gel of FIG. 15. Relative expression levels of constructs 15-28 (FIG. 14) are shown, as detected using anti-His antibodies.

FIG. 17 shows a table and images of a Coomassie blue-stained gel and a corresponding western blot. The table (bottom) provides the expected molecular weight (in kDa) (column 2) and the tag and/or fusion protein (column 3) associated with each construct of column 1 (constructs 29-32). The Coomassie blue-stained gel (left) shows proteins collected from the cell pellet (P) or supernatant (S) of E. coli cultures grown at 22° C. The western blot (right) shows relative expression levels of constructs 29-32 as detected using anti-His antibodies.

FIG. 18 shows a table that provides the expected molecular weight (in kDa) (column 2) and the tag and/or fusion protein (column 3) associated with each construct of column 1. Expression analysis results for constructs listed in FIG. 18 (constructs 33-47) are shown in the Coomassie blue-stained gel of FIG. 19 and the western blot of FIG. 20.

FIG. 19 provides an image of a Coomassie blue-stained gel showing proteins collected from the cell pellet (P) or supernatant (S) of E. coli cultures grown at 22° C. E. coli cultures each expressed one of constructs 33-47 (FIG. 18).

FIG. 20 shows an image of a western blot that corresponds to the Coomassie blue-stained gel of FIG. 19. Relative expression levels of constructs 33-47 (FIG. 18) are shown, as detected using anti-His antibodies.

FIG. 21 shows a table and images of a Coomassie blue-stained gel and a corresponding western blot. The table (bottom) provides the expected molecular weight (in kDa) (column 2) and the tag and/or fusion protein (column 3) associated with each construct of column 1 (constructs 48-54). The Coomassie blue-stained gel (left) shows proteins collected from the cell pellet (P) or supernatant (S) of E. coli cultures grown at 22° C. The western blot (right) shows relative expression levels of constructs 48-54 as detected using anti-His antibodies.

FIG. 22 shows a table that provides the expected molecular weight (in kDa) (column 2) and the tag and/or fusion protein (column 3) associated with each construct of column 1. Expression analysis results for constructs listed in FIG. 22 (constructs 5-14) are shown in the Coomassie blue-stained gel of FIG. 23 and the western blot of FIG. 24.

FIG. 23 provides an image of a Coomassie blue-stained gel showing proteins collected from the cell pellet (P) or supernatant (S) of E. coli cultures grown at 37° C. E. coli cultures each expressed one of constructs 5-14 (FIG. 22).

FIG. 24 shows an image of a western blot that corresponds to the Coomassie blue-stained gel of FIG. 23. Relative expression levels of constructs 5-14 (FIG. 22) are shown, as detected using anti-His antibodies.

FIG. 25 shows a table that provides the expected molecular weight (in kDa) (column 2) and the tag and/or fusion protein (column 3) associated with each construct of column 1. Expression analysis results for constructs listed in FIG. 25 (constructs 15-24) are shown in the western blot of FIG. 26.

FIG. 26 shows an image of a western blot. Relative expression levels of constructs 15-24 (FIG. 25) are shown, as detected using anti-His antibodies.

FIG. 27 shows a table that provides the expected molecular weight (in kDa) (column 2) and the tag and/or fusion protein (column 3) associated with each construct of column 1. Expression analysis results for constructs listed in FIG. 27 (constructs 25-37) are shown in the Coomassie blue-stained gel of FIG. 28 and the western blot of FIG. 29.

FIG. 28 provides an image of a Coomassie blue-stained gel showing proteins collected from the cell pellet (P) or supernatant (S) of E. coli cultures grown at 37° C. E. coli cultures each expressed one of constructs 25-37 (FIG. 27).

FIG. 29 shows an image of a western blot that corresponds to the Coomassie blue-stained gel of FIG. 28. Relative expression levels of constructs 25-37 (FIG. 27) are shown, as detected using anti-His antibodies.

FIG. 30 shows a table that provides the expected molecular weight (in kDa) (column 2) and the tag and/or fusion protein (column 3) associated with each construct of column 1. Expression analysis results for constructs listed in FIG. 30 (constructs 1-4, 38-39, and 41-48) are shown in the Coomassie blue-stained gel of FIG. 31 and the western blot of FIG. 32.

FIG. 31 provides an image of a Coomassie blue-stained gel showing proteins collected from the cell pellet (P) or supernatant (S) of E. coli cultures grown at 37° C. E. coli cultures each expressed one of constructs 1-4, 38-39, and 41-48 (FIG. 30).

FIG. 32 shows an image of a western blot that corresponds to the Coomassie blue-stained gel of FIG. 31. Relative expression levels of constructs 1-4, 38-39, and 41-4 (FIG. 30) are shown, as detected using anti-His antibodies.

FIG. 33 shows a table that provides the expected molecular weight (in kDa) (column 2) and the tag and/or fusion protein (column 3) associated with each construct of column 1. Expression analysis results for constructs listed in FIG. 30 (constructs 49-54) are shown in the Coomassie blue-stained gel and the western blot of FIG. 34.

FIG. 34 shows images of a Coomassie blue-stained gel and a corresponding western blot. The Coomassie blue-stained gel (left) shows proteins collected from the cell pellet (P) or supernatant (S) of E. coli cultures grown at 37° C. E. coli cultures each expressed one of constructs 49-54 (FIG. 33). The western blot (right) shows relative expression levels of constructs 9-54 as detected using anti-His antibodies.

### SEQUENCE LISTING

The nucleic acid and amino acid sequences listed in the accompanying sequence listing are shown using standard three letter codes for amino acids, and standard letter abbreviations for nucleotide bases, as defined in 37 C.F.R. § 1.822. Only one strand of each nucleic acid sequence is shown, but the complementary strand is understood as included by reference to the displayed strand. The Sequence Listing is submitted as an ASCII text file, created on Dec. 17, 2021, 798, 720 bytes, and is incorporated by reference herein.

SEQ ID NO. 1 is the genomic nucleic acid sequence of ASFV strain China/2018/AnhuiXCGQ.

SEQ ID NOs. 2-2273 are amino acid sequences of peptides associated with ASFV, particularly immunogenic peptides that stimulate an immune response to ASFV.

SEQ ID NOs. 2274-2291 are exemplary DNA sequences that can encode the 18 peptides of Appendix VI. The nucleic acid of SEQ ID NO. 2274 can encode the peptide of SEQ ID NO: 67. The nucleic acid of SEQ ID NO. 2275 can encode the peptide of SEQ ID NO: 69. The nucleic acid of SEQ ID NO. 2276 can encode the peptide of SEQ ID NO: 70. The nucleic acid of SEQ ID NO. 2277 can encode the peptide of SEQ ID NO: 279. The nucleic acid of SEQ ID NO. 2278 can encode the peptide of SEQ ID NO: 435. The nucleic acid of SEQ ID NO. 2279 can encode the peptide of SEQ ID NO: 461. The nucleic acid of SEQ ID NO. 2280 can encode the peptide of SEQ ID NO: 469. The nucleic acid of SEQ ID NO. 2281 can encode the peptide of SEQ ID NO: 478. The nucleic acid of SEQ ID NO. 2282 can encode the peptide of SEQ ID NO: 486. The nucleic acid of SEQ ID NO. 2283 can encode the peptide of SEQ ID NO: 547. The nucleic acid of SEQ ID NO. 2284 can encode the peptide of SEQ ID NO: 548. The nucleic acid of SEQ ID NO. 2285 can encode the peptide of SEQ ID NO: 549. The nucleic acid of SEQ ID NO. 2286 can encode the peptide of SEQ ID NO: 561. The nucleic acid of SEQ ID NO. 2287 can encode the peptide of SEQ ID NO: 589. The nucleic acid of SEQ ID NO. 2288 can encode the peptide of SEQ ID NO: 639. The nucleic acid of SEQ ID NO. 2289 can encode the peptide of SEQ ID NO: 652. The nucleic acid of SEQ ID NO. 2290 can encode the peptide of SEQ ID NO: 653. The nucleic acid of SEQ ID NO. 2291 can encode the peptide of SEQ ID NO: 1253. In each exemplary DNA sequence, the letter ‘R’ represents adenine or guanine; ‘K’ represents guanine or thymine; ‘H’ represents adenine, cytosine, or thymine; ‘D’ represents adenine, guanine, or thymine; ‘Y’ represents cytosine or thymine; ‘S’ represents cytosine or guanine; B represents cytosine, guanine, or thymine; ‘N’ represents adenine, guanine, cytosine, or thymine; ‘M’ represents adenine or cytosine; ‘W’ represents adenine or thymine; and ‘V’ represents adenine, cytosine, or guanine.

SEQ ID NOs. 2292-2309 are exemplary RNA sequences that can encode the 18 peptides of Appendix VI. The nucleic acid of SEQ ID NO. 2292 can encode the peptide of SEQ ID NO: 67. The nucleic acid of SEQ ID NO. 2293 can encode the peptide of SEQ ID NO: 69. The nucleic acid of SEQ ID NO. 2294 can encode the peptide of SEQ ID NO: 70. The nucleic acid of SEQ ID NO. 2295 can encode the peptide of SEQ ID NO: 279. The nucleic acid of SEQ ID NO. 2296 can encode the peptide of SEQ ID NO: 435. The nucleic acid of SEQ ID NO. 2297 can encode the peptide of SEQ ID NO: 461. The nucleic acid of SEQ ID NO. 2298 can encode the peptide of SEQ ID NO: 469. The nucleic acid of SEQ ID NO. 2299 can encode the peptide of SEQ ID NO: 478. The nucleic acid of SEQ ID NO. 2300 can encode the peptide of SEQ ID NO: 486. The nucleic acid of SEQ ID NO. 2301 can encode the peptide of SEQ ID NO: 547. The nucleic acid of SEQ ID NO. 2302 can encode the peptide of SEQ ID NO: 548. The nucleic acid of SEQ ID NO. 2303 can encode the peptide of SEQ ID NO: 549. The nucleic acid of SEQ ID NO. 2304 can encode the peptide of SEQ ID NO: 561. The nucleic acid of SEQ ID NO. 2305 can encode the peptide of SEQ ID NO: 589. The nucleic acid of SEQ ID NO. 2306 can encode the peptide of SEQ ID NO: 639. The nucleic acid of SEQ ID NO. 2307 can encode the peptide of SEQ ID NO: 652. The nucleic acid of SEQ ID NO. 2308 can encode the peptide of SEQ ID NO: 653. The nucleic acid of SEQ ID NO. 2309 can encode the peptide of SEQ ID NO: 1253. In each exemplary RNA sequence, the letter ‘R’ represents adenine or guanine; ‘K’ represents guanine or uracil; ‘H’ corresponds to adenine, cytosine, or uracil; ‘D’ represents adenine, guanine, or uracil; ‘Y’ represents cytosine or uracil; ‘S’ represents cytosine or guanine; B represents cytosine, guanine, or uracil; ‘N’ represents adenine, guanine, cytosine, or uracil; ‘M’ represents adenine or cytosine; ‘W’ represents adenine or uracil; and ‘V’ represents adenine, cytosine, or guanine.

SEQ ID NOs. 2310-2330 are constructs that can, for example, be expressed in a host cell using one or more plasmid vectors (such as a pHLT, pSumo, and/or pMBP) or viral vectors (such as a pseudorabies virus vector) or similar. Thus, each construct of SEQ ID NOs. 2310-2330 may further comprise an N-terminal fusion protein, such as HLT, Sumo, or MBP. Exemplary fusion protein sequences that can be attached to one or more construct of SEQ ID NOs. 2310-2330 are provided in SEQ ID NOs. 2336-2338. Further, each construct may comprise a His-tag, such as an N-terminal His-tag connected to either the N-terminus of the construct (if the construct does not include a fusion protein) or of the fusion protein attached to the construct. Constructs can also further comprise a C-terminal linker (GSSG) and HiBiT tag (GSGWRLFKKLS). For each construct, domains (areas of peptide clustering within ASFV proteins, also termed “hotspots” as described in Example 3, and provided individually as SEQ ID NOs. 2331-2335), full- and/or partial-length ASFV proteins (as provided in SEQ ID NOs. 2323-2329), and/or peptides of SEQ ID NOs. 2-2273 are provided in the order in which they appear in the construct sequence.

SEQ ID NO. 2310 comprises domains 10.1 and 1.1, and corresponds to constructs 1 and 2.

SEQ ID NO. 2311 comprises domains 3.1 and 11.1, and corresponds to constructs 3 and 4.

SEQ ID NO. 2312 comprises domains 10.1, 3.1d, 11.1, and 1.1, and corresponds to constructs 5 and 6.

SEQ ID NO. 1213 comprises domains 10.1, 3.1d, 1.1, and 11.1, and corresponds to constructs 7 and 8.

SEQ ID NO. 2314 comprises domain 10.1; peptides of SEQ ID NOs. 70, 478, 469, and 486; domain 3.1d; peptides of SEQ ID NOs. 547, 548, 549, 1253, and 279; and domains 1.1, 11.1, and corresponds to constructs 9,10, and 55.

SEQ ID NO. 2315 comprises peptides of SEQ ID NOs. 639, 548, 653, 589, 67, 69, 561, 461, 279, 547, 435, 478, 652, 486, 1253, 70, 469, and 549, and corresponds to constructs 11-14.

SEQ ID NO. 2316 comprises peptides of SEQ ID NOs. 639, 548, 653, 589, 67, 69, 561, 461, 279, 547, 435, 478, 652, 486, 1253, 70, 469, and 549, with a spacer (GPGPG) separating each individual peptide sequence, and corresponds to constructs 15-18.

SEQ ID NO. 2317 comprises peptides of SEQ ID NOs. 639, 548, 653, 589, 67, 69, 561, 461, 279, 547, 435, 478, 652, 486, 1253, 70, 469, and 549, with a spacer (AAY) separating each individual peptide sequence, and corresponds to constructs 19-22.

SEQ ID NO. 2318 comprises peptides of SEQ ID NOs. 639, 548, 653, 589, 67, 69, 486, 561, 461, 279, 547, 435, 478, 1253, 70, 652, 469, and 549, corresponding to constructs 23-26.

SEQ ID NO. 2319 comprises peptides of SEQ ID NOs. 639, 548, 653, 589, 67, 69, 486, 561, 461, 279, 547, 435, 478, 1253, 70, 652, 469, and 549, with a spacer (GPGPG) separating each individual peptide sequence, and corresponds to constructs 27-30.

SEQ ID NO. 2320 comprises peptides of SEQ ID NOs. 639, 548, 653, 589, 67, 69, 486, 561, 461, 279, 547, 435, 478, 1253, 70, 652, 469, and 549, with a spacer (GPGPG) separating each individual peptide sequence, and corresponds to constructs 31-34.

SEQ ID NO. 2321 comprises peptides of SEQ ID NOs. 478, 279, 652, 1253, 469, 363, 462, 377, 400, 187, 404, 461, 463, 496, 589, 70, 486, 32, 278, 128, 435, 653, 456, 492, 561, 548, 468, 67, 447, 549, 449, 69, 639, 547, 455, 467, 101 and 457, and corresponds to constructs 35-37.

SEQ ID NO. 2322 comprises peptides of SEQ ID NOs. 478, 279, 652, 1253, 469, 363, 462, 377, 400, 187, 404, 461, 463, 496, 589, 70, 486, 32, 278, 128, 435, 653, 456, 492, 561, 548, 468, 67, 447, 549, 449, 69, 639, 547, 455, 467, 101, 457, 640, 645, 670, 553, 711, 662, 621, 633, 651, 541, 584, 529, 497, 544, 527, 636, 578, 619, 554, 1156, 1248, 1280, 1288, 1440, 2021, 2204, 1561, 1437, 1106, 1584, 1556, 1560, 743, 1531, 2112 and 1049, with a spacer (GPGPG) separating peptides 101 and 457, and with a spacer (AAY) separating peptides 619 and 554, and corresponds to constructs 38-40.

SEQ ID NO. 2323 comprises the ASFV protein of GenBank Accession No. AYW33963.1, and corresponds to constructs 41 and 48.

SEQ ID NO. 2324 comprises the ASFV protein of GenBank Accession No. AYW34001.1, and corresponds to constructs 42 and 49.

SEQ ID NO. 2325 comprises the ASFV protein of GenBank Accession No. AYW34002.1, and corresponds to constructs 43 and 50.

SEQ ID NO. 2326 comprises the ASFV protein of GenBank Accession No. AYW34004.1, and corresponds to constructs 44 and 51.

SEQ ID NO. 2327 comprises the ASFV protein of GenBank Accession No. AYW34010.1, and corresponds to constructs 45 and 52.

SEQ ID NO. 2328 comprises the ASFV protein of GenBank Accession No. AYW34011.1, and corresponds to constructs 46 and 53.

SEQ ID NO. 2329 comprises the ASFV protein of GenBank Accession No. AYW34052.1, and corresponds to constructs 47 and 54.

SEQ ID NO. 2330 is construct 56, which comprises the peptides of SEQ ID NOs. 639,548, 653, 589, 67, 69, 561, 461, 279, 547, 435, 478, 652, 486, 1253, 70, 469 and 549, with GPGPG spacer sequences between peptides 653 and 589, 652 and 486, and 1253 and 70and with AAY spacer sequences between 69 and 56, 279 and 547 peptides sequences.

SEQ ID NO. 2331 is domain 1.1.

SEQ ID NO. 2332 is domain 3.1.

SEQ ID NO. 2333 is domain 3.1d.

SEQ ID NO. 2334 is domain 10.0.

SEQ ID NO. 2335 is domain 11.1.

SEQ ID NO. 2336 is an exemplary Sumo fusion protein.

SEQ ID NO. 2337 is an exemplary MBP fusion protein.

SEQ ID NO. 2338 is the lipoyl domain from Bacillus stearothermophilus E2p, which is included in whole or in part the HLT fusion protein.

SEQ ID NO. 2339 is a nucleotide sequence encoding the ASFV protein of GenBank Accession No. AYW33963.1.

SEQ ID NO. 2340 is a nucleotide sequence encoding the ASFV protein of GenBank Accession No. AYW34001.1.

SEQ ID NO. 2341 is a nucleotide sequence encoding the ASFV protein of GenBank Accession No. AYW34002.1.

SEQ ID NO. 2342 is a nucleotide sequence encoding the ASFV protein of GenBank Accession No. AYW34004.1.

SEQ ID NO. 2343 is a nucleotide sequence encoding the ASFV protein of GenBank Accession No. AYW34010.1.

SEQ ID NO. 2344 is a nucleotide sequence encoding the ASFV protein of GenBank Accession No. AYW34011.1.

SEQ ID NO. 2345 is a nucleotide sequence encoding the ASFV protein of GenBank Accession No. AYW34052.1.

### DETAILED DESCRIPTION

Identification of ASFV cytotoxic T lymphocyte (CTL) epitopes relevant for inducing protective immunity in swine by vaccination is challenging in part due to the heterogeneity of the T cell population and to variations in swine leukocyte antigen (SLA) class I antigen-binding specificities. However, effective vaccines are needed to reduce the spread and impact of ASF in swine populations.

The ASFV genome includes a conserved central region (CCR) and both left and right variable regions, each of which contains different numbers of five multigene family (MGF) genes. CCR gene products are involved in viral replication and assembly as well as in modulating immune evasion and host cellular functions. Variability among ASFV genomes results primarily from MGF member loss or gain.

Swine can survive infection with less-virulent isolates of ASFV and may become chronically infected. Surviving animals are resistant to challenge with related isolates of the virus, indicating that domestic swine can develop protective immunity against ASFV. During asymptomatic, non-virulent ASFV infections, natural killer cell activity increases in swine, suggesting that this cell type plays a role in ASFV immunity. Further, CD8+lymphocyte depletion from ASFV immune swine abrogates protective immunity against related virulent viruses. This suggests that the presence of ASFV-specific antibodies alone is insufficient to protect against ASFV infection and that the CD8+ lymphocyte subset plays an important role in ASFV protective immunity.

The present disclosure concerns immunogenic peptides, and compositions comprising such peptides. The disclosed peptides are used to form immunogenic peptide compositions, and/or nucleic acid-, viral or bacterial vector-, or host cell-based vaccines, and/or combinations thereof, that elicit or stimulate an immune response against ASFV. Such immunogenic compositions can be administered to an animal in combination with additional therapeutics, such as compounds or compositions aimed at reducing or alleviating the symptoms of ASF, or other compositions such as vaccines against other infections common in swine.

### I. Abbreviations

ASF African swine fever

ASFV African swine fever virus

CCID⁵⁰ Cell culture infectious dose 50%

CCR Conserved central region

CTL Cytotoxic T lymphocyte

dpv Days post (initial) vaccination

ELISA Enzyme-linked immunosorbent assay

ELISpot Enzyme-linked immunosorbent spot assays

INF-γ Interferon-gamma

MDA Maternally-derived antibody

MGF Multi-gene family

MHC Major histocompatibility complex

MS Mass spectrometry

PBMC Peripheral blood macrophage cell

PCR Polymerase chain reaction

qPCR Quantitative polymerase chain reaction

SLA Swine leukocyte antigen

### II. Terms and Definitions

Unless otherwise noted, technical terms are used according to conventional usage as would be understood by a person of ordinary skill in the art. Definitions of common terms in molecular biology may be found in Lewin's Genes X, ed. Krebs et al, Jones and Bartlett Publishers, 2009 (ISBN 0763766321); Kendrew et al. (eds.), The Encyclopedia of Molecular Biology, Blackwell Publishers, 1994 (ISBN 0632021829); Robert A. Meyers (ed.), Molecular Biology and Biotechnology: A Comprehensive Desk Reference, Wiley, John & Sons, Inc., 1995 (ISBN 0471186341); and George P. Rédei, Encyclopedic Dictionary of Genetics, Genomics, Proteomics and Informatics, 3rd Edition, Springer, 2008 (ISBN: 1402067534).

The following explanations of terms and abbreviations are provided to better describe the present disclosure and to guide those of ordinary skill in the art to practice the present disclosure. As used herein, “comprising” means “including” and the singular forms “a” or “an” or “the” refer to one or more than one unless the context clearly dictates otherwise. The term “or” refers to a single element of stated alternative elements or a combination of two or more elements, unless the context clearly indicates otherwise.

All publications, patent applications, patents, and other references mentioned herein are incorporated by reference in their entirety for all purposes. All sequences associated with the GenBank Accession Nos. mentioned herein are incorporated by reference in their entirety as of the present application's priority date. In case of conflict, the present specification, including explanations of terms, will control.

Although methods and materials similar or equivalent to those described herein can be used to practice or test the present disclosure, suitable methods and materials are described below. The materials, methods, and examples are illustrative only and not intended to be limiting. Other features of the disclosure will be apparent to a person of ordinary skill in the art from the following detailed description and the claims.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is recited.

Amino acid residues in the disclosed sequence listing may be conservatively substituted or replaced by another residue with similar properties and characteristics. Typically, conservative substitutions have little to no impact on the activity of a resulting peptide. In one non-limiting example, a tyrosine residue in one peptide of a composition is substituted with a tryptophan residue. A peptide can be produced by chemical substitution to include one or more conservative amino acid substitutions, or can be produced by manipulating the nucleic acid sequence that encodes that peptide using, for example, standard procedures such as PCR or site-directed mutagenesis. Table 1 below provides conservative amino acid substitutions for expressly disclosed peptide sequences that are within the scope of the present disclosure.

TABLE 1
Conservative Amino Acid Substitutions

| Definition | Amino Acid | Symbol |
|---|---|---|
| Amino acids with aliphatic R- | Glycine | Gly-G |
| groups | Alanine | Ala-A |
| | Valine | Val-V |
| | Leucine | Leu-L |
| | Isoleucine | Ile-I |
| Amino acids with hydroxyl R- | Serine | Ser-S |
| groups | Threonine | Thr-T |
| Amino acids with sulfur-containing | Cysteine | Cys-C |
| R-groups | Methionine | Met-M |
| Acidic amino acids | Aspartic Acid | Asp-D |
| | Asparagine | Asn-N |
| | Glutamic Acid | Glu-E |
| | Glutamine | Gln-Q |
| Basic amino acids | Arginine | Arg-R |
| | Lysine | Lys-K |
| | Histidine | His-H |
| Amino acids with aromatic rings | Phenylalanine | Phe-F |
| | Tyrosine | Tyr-Y |
| | Tryptophan | Trp-W |
| Imino acids | Proline | Pro-P |

To facilitate review of the various embodiments of this disclosure, the following explanations of specific terms are provided:

Adjuvant: The term “adjuvant” as used herein means any substance or vehicle that enhances the effectiveness of a disclosed immunogenic composition, such as by enhancing the immune response to an antigen (for example an ASFV antigen) by an animal's immune system, such as a mammalian immune system. An adjuvant can be used to form a composition or compositions disclosed herein, for example as part of an ASFV vaccine composition. Adjuvants included in some embodiments of a composition disclosed herein can include, but are not limited to, aluminum salts, such as aluminum phosphate or aluminum hydroxide; various types of oils, such as vegetable oil, mineral oil, or cinnamon oil (See U.S. Patent No. 2006/0275515, “Antiviral preparations obtained from a natural cinnamon extract,” which is incorporated by reference herein); oil-in-water based adjuvants, such as Emulsigen®, Emulsigen®-D, Emulsigen®-DL90, Emulsigen®-P, Emulsigen®-BCL, Emulsimune®, or TS6; Amphigen®; pluronic polyols; saponin-based adjuvants, such as saponin, Quil A, and QS-21; nonionic block copolymers; microfluidized emulsions, such as 1MF59; water-in-oil adjuvants, such as ISA 720, ISA 71 VG, ISA 35, ISA 51, or ISA 50V; water-in-oil-in-water based adjuvants, such as ISA 206 or ISA 201 (such as Montanide ISA 201 VG); Freund's complete adjuvant; Freund's incomplete adjuvant; polylactide glycolide (PLGA); toll-like receptor (TLR) ligand-based adjuvants, such as TLR7/8 adjuvants, such as R848 (Resiquimod); Carbomer-based adjuvants, such as those containing 934P or 971P; polymer-based adjuvants, such as Carbigen™or PolygenTM; immune-stimulating complexes (ISCOMs); liposomes; polysaccharides; derivatized polysaccharides; oligonucleotides; cytokines; bacterial derivatives, such as trehalose-6,6-dibehenate (TDB) or cyclic diguanylate monophosphate (c-di-GMP); viral derivatives, such as polyinosinic-polycitidylic acid (poly (I:C)); or combinations thereof.

“Mucosally-adjuvanted” or “mucosal adjuvant” refer to an adjuvant or other compound, such as, for example, a polymer, that can interact with mucosal membranes and may stimulate an immune response. Additional information concerning mucosal adjuvants is provided by U.S. Patent No. 10,279,031, which is incorporated by reference herein. Mucous membranes include the optic (eye), oral, nasopharyngeal, anal, or vaginal membranes. The immune response that may be stimulated may include IgM, IgG, IgA, or a combination thereof. Compositions comprising such adjuvants may be applied to the mucosal membranes of an animal. Mucosal adjuvants may be “mucoadhesive,” in that they may adhere (generally non-covalently) to a mucosal membrane. Specific adjuvants with mucoadhesive properties include, but are not limited to, adjuvants comprising polymers, such as those comprising polyacrylic acids, such as Carbomers and Carbopols, or oil-in-water based adjuvants. Additionally, adjuvants containing nanoparticles may be used for intranasal administration. A person of ordinary skill in the art understands that a mucoadhesive adjuvant may contain one or a combination of any of the above adjuvants.

Administer, Administering, Administration: As used herein, administering a composition (e.g. an immunogenic composition) to an animal means to apply, give, or bring the composition into contact with the animal. Administration can be accomplished by a variety of routes, such as, for example, topical, oral, subcutaneous, transdermal, intrathecal, intramuscular, intravenous, intraperitoneal, intranasal, and similar routes, or combinations thereof.

As used herein, administering a composition mucosally includes directly administering the composition to an animal, such as by directly placing, such as, for example, spraying and/or dropping, the composition in the animal's mouth, nasal passages, or eye. Administering the composition mucosally also comprises providing the composition such that the animal administers the composition to itself, such as providing a composition for the animal to ingest. Exemplary methods of providing the composition include, but are not limited to, spraying the composition on the animal and/or otherwise topically applying the composition to the skin, or providing the composition in a form that the animal will eat. A person of ordinary skill in the art will understand that spraying may also facilitate direct administration because spray droplets may directly enter the mouth, nasal cavity, and/or eye of a swine. Another exemplary method of administering the composition to an animal is by intramuscular administration, such as, for example, by injection of a liquid formulation of the composition.

Disclosed compositions may be formulated for parenteral administration, such as, for example, by intradermal, intraarterial, intraperitoneal, intramuscular, subcutaneous, or intravenous routes, or combinations thereof. Examples of parenteral formulations of the compositions include, but are not limited to, suspensions that can be injected, solutions that can be injected, emulsions, and dry products that can be dissolved or suspended in an acceptable vehicle for injection. In addition, controlled-release parenteral formulations of the compositions can be prepared or administered, or both. Suitable materials for such administration include alcohols or a mixture or alcohols, such as a C₁-C₁₀ alcohol, such as ethanol, propanol, butanol, pentanol, hexanol, heptanol, octanol, nonanol, and/or decanol; polyols, such as polyethylene glycol; sterile water; glucose solution; saline solution; aqueous vehicles, such as, but not limited to, sodium chloride, dextrose, Dextrose Injection, Sodium Chloride Injection, Ringer's Injection, or Lactated Ringer's Injection, or combinations thereof non-aqueous vehicles such as, but not limited to, ethyl oleate, peanut oil, corn oil, cottonseed oil, sesame oil, or isopropyl myristate, or combinations thereof aqueous and non-aqueous isotonic sterile injection solutions, which can contain bacteriostats, buffers, antioxidants, or solutes that render the formulation isotonic within the blood of the recipient, or combinations thereof; and non-aqueous and aqueous suspensions that can be sterile and can include solubilizers, stabilizers, thickening agents, suspending agents, and preservatives, or combinations thereof. Formulations of the compositions can be presented in unit-dose or multi-dose containers, such as bottles, ampules, syringes, tubes, capsules, and vials.

African Swine Fever (ASF): “African swine fever” is caused by ASFV and typically presents as hemorrhagic fever. ASF is a highly contagious and deadly disease affecting both domestic and wild swine worldwide, with a mortality rate approaching 100% in domestic swine.

African Swine Fever Virus (ASFV): “African swine fever virus” is a virus that causes ASF in swine. The virus can be transmitted by ingestion, contact, or through ticks of the genus Ornithodoros. ASFV is the only member of the Asfarviridae family and has a linear, double-stranded DNA genome. In certain embodiments, the ASFV genome is 170-193 kbp and encodes 151-167 genes. The ASFV genome includes a conserved central region (CCR) of approximately 125 kbp and both left and right variable regions that each contain different numbers of five multigene family (MGF) genes. CCR gene products are involved in viral replication and assembly, and in modulating immune evasion and host cellular functions. Variability among ASFV genomes results primarily from MGF member loss or gain.

Multiple strains of ASFV have been identified, and nucleic acid sequences for ASFV are publicly available. For example, the ASFV strain identified as Ken06.Bus (GenBank Accession No. KM111295.1; incorporated by reference as present in GenBank as of the present application's priority date) provides an exemplary ASFV genome sequence.

Animal: “Animal” refers to a living multi-cellular vertebrate organism, a category that includes, for example, mammals and birds. The term mammal includes both human and non-human mammals, such as ungulates, and particularly swine. “Swine” (also referred to herein as “pigs”) includes members of genus Sus, such as Sus scrofa, such as Sus scrofa domesticus, such as the Yorkshire, Duroc, and/or Landrace swine breeds.

Antibody: An “antibody” is an immunoglobulin molecule produced by B lymphoid cells. Antibodies are evoked in humans or other animals by a specific antigen (immunogen). Antibodies are characterized by reacting specifically with the antigen in some demonstrable way. “Eliciting an antibody response” refers to the ability of an antigen or other molecule to induce the production of antibodies.

Antigen: “Antigen” refers to a compound, composition, or substance that can stimulate the production of antibodies or a T-cell response in an animal, including compositions that are injected or absorbed into an animal.

Viral antigens suitable for use in the present technology include inactivated (or killed) virus and/or a viral peptide, peptides, protein, or proteins, that may be isolated, purified or derived from a virus. Viral antigens can be derived from viruses propagated on a substrate, such as a cell culture or other substrate, or they may be derived or expressed recombinantly, or they may be synthesized. Typically, viral antigens include, but are not limited to, epitopes which are exposed on the surface of the virus during at least one stage of a life cycle. Viral antigens may be conserved across multiple serotypes or isolates. Viral antigens include antigens derived from one or more of the viruses disclosed herein.

Attenuated, Attenuation: An “attenuated” virus is a virus that is weakened and/or less virulent as compared to a non-attenuated form of the virus, which may be capable of causing disease. Attenuated viruses may stimulate an immune response and/or immunity but are not capable of causing disease. Replication of an attenuated virus in culture and/or a recipient may be the same as, similar to, or different from that of a strain or strains from which the attenuated virus was derived. Attenuation may be achieved by altering a virus using one or more methods that involve a single step and/or multiple steps. For example, attenuating genetic modifications, such as, for example, attenuating mutations and/or genetic reassortment, may be introduced into coding and/or non-coding regions of a viral genome through site-directed mutagenesis, chemical methods, irradiation, and/or recombinant techniques. Such methods are well known to those of ordinary skill in the art. An attenuated form of an otherwise disease-causing virus may also be identified through culturing techniques, such as passaging, and/or may result from genetic differences in a viral genome not induced, created, or caused by human intervention. Methods of determining whether an attenuated virus maintains similar or reduced antigenicity as compared to the strain or strains from which the attenuated virus was derived are also well known to those of ordinary skill in the art. Such methods may include, for example, chemical selection and/or nucleic acid screening, such as, for example, by probe hybridization or PCR. Attenuated viruses, such as, for example, certain embodiments of viral vectors disclosed herein, may be used to stimulate an immune response and/or induce immunity in a recipient, such as an animal, such as a swine.

Cinnamon: The term “cinnamon” refers to a product or products, such as, for example a cinnamon extract, a fraction of a cinnamon extract, and/or a precipitate of a cinnamon extract, derived from one or more members of the Cinnamomum genus. Such members may include, for example, C. zeylanicum, C. cassia (C. aromaticum), C. camphora, C. burmannii, C. verum, C. loureiroi, C. citriodorum, C. dubium, C. japonicum, C. kanehirae, C. virens, C. tamala, C. parthenoxylon, C. mercadoi, C. glaucescens, C. malabatrum, C. cambodianum, any other member of genus Cinnamomum, or combinations thereof. Typically, the one or more products is derived from the bark and/or leaves of one or more members of the Cinnamomum genus by one or more appropriate extraction, fractionation, and/or precipitation methods and/or similar methods. Combination: A combination includes two or more components that are administered such that the effective time period of at least one component overlaps with the effective time period of at least one other component. A component may be a composition. In some embodiments, the effective time periods of all components administered overlap with each other. In an exemplary embodiment of a combination comprising three components, the effective time period of the first component administered may overlap with the effective time periods of the second and third components, but the effective time period of the second component independently may or may not overlap with that of the third component. In an exemplary embodiment of a combination comprising four components, the effective time period of the first component administered overlaps with the effective time periods of the second, third, and fourth components; the effective time period of the second component overlaps with those of the first and fourth components, but not that of the third component; and the effective time period of the fourth component overlaps with that of the second and third components only. A combination may be a composition comprising the components, a composition comprising two or more individual components, or a composition comprising one or more components and another separate component (or components) or composition(s) comprising the remaining component(s). In some embodiments, the two or more components may comprise two or more different components administered substantially simultaneously or sequentially in any order, the same component administered at two or more different times, or a combination thereof.

Conditions sufficient for: The term “conditions sufficient for” refers to any environment that permits the desired activity, for example, that permits specific binding or hybridization between two nucleic acid molecules or that permits amplification and/or detection of a nucleic acid. Such an environment may include, but is not limited to, particular incubation conditions (such as time and/or temperature) or presence and/or concentration of particular factors, for example in a solution (such as buffer(s), salt(s), metal ion(s), detergent(s), nucleotide(s), enzyme(s), and so on).

Effective amount: The term “effective amount” or “therapeutically effective amount” or “immune-stimulatory amount” refers to the amount of an agent (such as one or more embodiments provided herein alone, in combination, or potentially in combination with other therapeutic agent(s)) that is sufficient to induce a desired biological result. That result may be amelioration or alleviation of the signs, symptoms, or causes of a disease, or any other desired alteration of a biological system. The amount can vary with the condition being treated, the stage of advancement of the condition, and the type and concentration of formulation applied. In some embodiments, an effective amount of an immune stimulatory composition is an amount which, when administered to a subject, is sufficient to engender a detectable immune response. Such a response may comprise, for instance, generation of an antibody specific to one or more of the epitopes provided in the immune stimulatory composition. Alternatively, the response may comprise a T-helper or CTL-based response to one or more of the epitopes provided in the immune stimulatory composition. All three of these responses may originate from naive or memory cells. In other embodiments, a “protective effective amount” of an immune stimulatory composition is an amount which, when administered to a subject, is sufficient to confer protective immunity to the subject. Appropriate amounts in any given instance will be readily apparent to those of ordinary skill in the art or capable of determination by routine experimentation such as vaccination and observation of an antibody response or vaccination followed by a challenge wherein the vaccinated animal performs better than a non-vaccinated animal that is challenged similarly.

Encoding: “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (for example, rRNA, tRNA, and mRNA) or a defined sequence of amino acids and the biological properties resulting therefrom. Thus, a gene encodes a protein if transcription and translation of mRNA produced by that gene is capable of producing the protein, such as in a cell or other biological system. Both the coding strand, the nucleotide sequence of which is identical to the mRNA sequence and is usually provided in sequence listings, and noncoding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns, exons, or both.

Epitope: An “epitope” is an antigenic determinant. These are chemical groups or peptide sequences on a molecule that are antigenic, i.e. that elicit an immune response. T cell epitopes are presented on the surface of an antigen-presenting cell, where they are bound to WIC molecules. Professional antigen-presenting cells, such as macrophages, dendritic cells, and B cells, are specialized to present WIC class II peptides, whereas most nucleated somatic cells present WIC class I peptides. T cell epitopes presented by WIC class I molecules are typically peptides between 8 and 11 amino acids in length, whereas WIC class II molecules present longer peptides, 13-17 amino acids in length. An antibody specifically binds a particular antigenic epitope on a peptide, such as one or more immunogenic peptides selected from SEQ ID NOs. 2-2273. In some examples a disclosed peptide is an epitope.

Expression: “Expression” refers to transcription and/or translation of a nucleic acid sequence. For example, a gene can be expressed when its DNA is transcribed into an RNA or RNA fragment, which in some examples is processed to form mRNA. A gene may also be expressed when its mRNA is translated into an amino acid sequence, such as a protein or a protein fragment. In a specific example, a heterologous gene is expressed when it is transcribed into an RNA. In another specific example, a heterologous gene is expressed when its RNA is translated into an amino acid sequence. Regulation of expression can include controls on transcription, translation, RNA transport and processing, degradation of intermediary molecules such as mRNA, or through activation, inactivation, compartmentalization or degradation of specific protein molecules after they are produced.

Expression Control Sequences: “Expression control sequences” are nucleic acid sequences that regulate the expression of a heterologous nucleic acid sequence to which they are operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

Expression vector: An “expression vector” is a vector comprising a recombinant polynucleotide comprising expression control sequences operatively linked to a nucleotide sequence to be expressed. An expression vector comprises sufficient elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

Host cell: “Host cell” refers to a cell or cells in which a vector can be propagated and its DNA expressed. The cell can be eukaryotic or prokaryotic. The cell can be mammalian, such as a swine cell. “Host cell” also includes any progeny of the subject host cell. It is understood that all progeny may or may not be identical to the parental cell since mutations may occur during replication. Such progeny are understood to be included when the term “host cell” is used.

Immune response: An “immune response” is a response of a cell of the immune system, such as a B-cell, T-cell, macrophage or polymorphonucleocyte, to a stimulus, such as an antigenic peptide. An immune response can include any cell of the body involved in a host defense response, including for example, an epithelial cell that secretes an interferon or a cytokine. An immune response includes, but is not limited to, an innate immune response or inflammation. As used herein, a protective immune response refers to an immune response that protects a subject from infection (prevents infection or prevents the development of disease associated with infection). Methods of measuring immune responses are known to those of ordinary skill in the art and include, for example, measuring proliferation and/or activity of lymphocytes (such as B or T cells), secretion of cytokines or chemokines, inflammation, antibody production and the like.

Immune stimulatory composition: The terms, “immune stimulatory composition” and “immunogenic composition” used herein mean a composition useful for stimulating or eliciting an immune response (or immunogenic response) in a subject. The immune stimulatory composition can be a protein antigen, a nucleic acid molecule (such as vector) used to express a protein antigen, or a combination thereof. In some embodiments, the immunogenic response is protective or provides protective immunity, in that it enables the subject to better resist infection with or disease progression from the virus against which the immune stimulatory composition is directed.

Immunize: To render a subject (such as a mammal, and particularly swine) protected, through stimulation of the subject's immune system (such as by vaccination), from infection by an infectious disease (such as ASFV).

Immunogen: A compound, composition, or substance that can stimulate an immune response, such as the production of antibodies or a T-cell response in an animal, including compositions that are injected or absorbed into an animal. Particular non-limiting examples of immunogens include immunogenic peptides of SEQ ID NOs. 2-2273, constructs of SEQ ID NOs. 2310-2330, domains of SEQ ID NOs: 2331-2335, and/or full- and/or partial-length ASFV proteins (for example, one or more proteins of SEQ ID NOs: 2323-2329), and/or nucleic acids, vectors, and/or host cells encoding such peptides, constructs, domains, and/or full- and/or partial-length ASFV proteins.

Inactivated: In the context of the present disclosure, an “inactivated” virus is one that has been altered to the extent that it not capable of establishing an infection in a host or host cell. Viruses can be inactivated using, for example, chemicals, heat, alterations in pH and/or irradiation (such as ultraviolet or gamma irradiation). Inactivated viruses are also referred to as “killed.” A “chemically inactivated” virus is a virus that has been inactivated using a chemical method, such as treatment with betapropiolactone, formaldehyde, glutaraldehyde, 2,2′-dithiodipyridine or binary ethylene imine. For a review of inactivation methods for virus vaccines, see Delrue et al. (Expert Rev Vaccines 11(6):695-719, 2012).

Infection: Infection or challenge means that the subject has been exposed to organisms that may produce disease causing the subject to suffer one or more clinical signs of the diseases when they have been exposed to such organisms.

Isolate, Isolated: An “isolated” biological component (such as a nucleic acid) has been substantially separated or purified away from biological or other components (for example biological components with which the component naturally occurs, such as chromosomal and extrachromosomal DNA, RNA, and proteins). Nucleic acids that have been “isolated” include nucleic acids purified by standard purification methods. The term also embraces nucleic acids prepared by recombinant expression in a host cell and subsequently purified, as well as chemically synthesized and purified nucleic acid molecules. Isolated does not require absolute purity, and can include, for example, nucleic acid molecules wherein at least 50%, 60%, 70%, 75%, 80%, 85%, 90%, 95%, 98%, 99%, or 99.9% of components in the original mixture with the desired materials are removed. As another example, an isolated biological component is one in which the biological component is more enriched than the biological component is in its natural environment within a cell, or other production vessel. An isolated nucleic acid may be in solution (e.g., water or an aqueous solution) or dried.

Peptide: A “peptide” is a polymer having at least two amino acids joined by a peptide bond, and more typically more than 2 amino acids joined together by amide bonds. Certain peptides, such as peptides having 25 or more amino acids, may be referred to as polypeptides. When the amino acids are alpha-amino acids, the L-optical isomer, the D-optical isomer, or combinations thereof, can be used. The term “peptide” as used herein is intended to encompass any amino acid sequence and includes modified sequences such as glycoproteins, and covers naturally occurring amino acid sequences, as well as those that are recombinantly or synthetically produced. The term “residue” or “amino acid residue” refers to an amino acid that is incorporated into a peptide. Exemplary peptides disclosed herein include the peptides of SEQ ID NOs. 2-2273, constructs of SEQ ID NOs. 2310-2330, domains of SEQ ID NOs: 2331-2335, and ASFV proteins, for example, of SEQ ID NOs. 2323-2329.

Polynucleotide, Nucleic Acid Molecule: The term “nucleic acid molecule” or “polynucleotide” refers to a polymeric form of nucleotide of at least two bases in length, unless otherwise specified. A nucleic acid molecule may include both sense and anti-sense strands of cDNA, genomic DNA, RNA, and/or mixed polymers and/or synthetic forms of the above. The term “nucleic acid molecule” as used herein is synonymous with “nucleic acid” and “polynucleotide.” The terms include single- and double-stranded forms of DNA, unless specified otherwise. A polynucleotide may include either or both naturally occurring nucleotides and modified nucleotides linked together by naturally occurring and/or non-naturally occurring nucleotide linkages. The nucleotides can be ribonucleotides, deoxyribonucleotides, or modified forms of either nucleotide.

A recombinant polynucleotide includes a polynucleotide that is not immediately contiguous with both of the coding sequences with which it is immediately contiguous (one on the 5′ end and one on the 3′ end) in the naturally occurring genome of the organism from which it is derived. A recombinant nucleic acid molecule can also be one that is not naturally occurring or has a sequence that is made by an artificial combination of two otherwise separated segments of sequence. This artificial combination is accomplished by chemical synthesis or by artificial manipulation of isolated segments of nucleic acids, such as, for example, by genetic engineering techniques known to those of ordinary skill in the art. The term therefore includes, for example, a recombinant DNA molecule that is incorporated into a vector; into an autonomously replicating plasmid or virus; or into the genomic DNA of a prokaryote or eukaryote, or which exists as a separate molecule (for example, a cDNA) independent of other sequences.

Preventing: Preventing a disease refers to inhibiting the full development of a disease.

Treating: Refers to a therapeutic intervention that ameliorates a sign or symptom of a disease or pathological condition after it has begun to develop.

Ameliorating: Refers to a reduction in the number or severity of one or more signs or symptoms of a disease.

Promoter: A “promoter” is a minimal nucleic acid sequence sufficient to direct transcription. A promoter is typically located in the 5′ region adjacent to (and upstream of) the transcriptional start site of a gene, and generally contains a functional TATA box that directs the expression of the gene. A promoter generally contains both structural and functional elements and provides a control point for regulating the transcription of the associated gene. Also included are those promoter elements that are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et al., Methods in Enzymology 153:516-544, 1987). For example, when cloning in bacterial systems, inducible promoters such as pL of bacteriophage lambda, plac, ptrp, ptac (ptrp-lac hybrid promoter) and the like may be used. In one embodiment, when cloning in mammalian cell systems, promoters derived from the genome of mammalian cells (such as metallothionein promoter) or from mammalian viruses (such as the retrovirus long terminal repeat; the adenovirus late promoter; the vaccinia virus 7.5K promoter) can be used. Promoters produced by recombinant DNA or synthetic techniques may also be used to provide for transcription of nucleic acid sequences.

A polynucleotide can be inserted into an expression vector that contains a promoter sequence, which facilitates the efficient transcription of the inserted genetic sequence of the host. The expression vector typically contains an origin of replication, a promoter, as well as specific nucleic acid sequences that allow phenotypic selection of transformed cells.

Purified: The term “purified” does not require absolute purity; rather, it is intended as a relative term. Thus, for example, a purified protein, virus, nucleic acid, or other compound is one that is isolated in whole or in part from associated proteins and other contaminants. In certain embodiments, the term “substantially purified” refers to a protein, virus, nucleic acid, or other compound that has been isolated from a cell, cell culture medium, or other crude preparation and subjected to purification to remove various components of the initial preparation, such as proteins, cellular debris, and other components.

Recombinant: A recombinant nucleic acid, protein, or virus is one that has a sequence that is not naturally occurring or has a sequence that is made by an artificial combination of two otherwise separated sequence segments. This artificial combination is often accomplished by chemical synthesis or, more commonly, by manipulating isolated segments of nucleic acids, for example, by genetic engineering techniques. The term recombinant includes nucleic acids, proteins and viruses that have been altered solely by addition, substitution, or deletion of a portion of a natural nucleic acid molecule, protein or virus.

Sample: A “sample” (or “biological sample”) refers to a specimen obtained from an organism, comprising, in certain embodiments, DNA (for example, genomic DNA or cDNA), RNA (including mRNA), protein, or combinations thereof. Examples include, but are not limited to isolated nucleic acids, cells, proteins, peptides, cell lysates, chromosomal preparations, tissues, and bodily fluids (such as blood, derivatives and fractions of blood (such as serum)), extracted galls, biopsied or surgically removed tissue (including tissues that are, for example, unfixed, frozen, fixed in formalin and/or embedded in paraffin), autopsy material, tears, milk, skin scrapes, surface washings, urine, sputum, cerebrospinal fluid, prostate fluid, pus, bone marrow aspirates, middle ear fluids, bronchoalveolar lavage, tracheal aspirates, nasopharyngeal swabs or aspirates, oropharyngeal swabs or aspirates, nasal washings, or saliva. In one example, a sample includes viral peptides, for example, specific to ASFV. In particular examples, samples are used directly (e.g., fresh or frozen), or can be manipulated prior to use, for example, by extraction (for example of nucleic acids), fixation (e.g., using formalin) and/or embedding in wax (such as formalin-fixed paraffin-embedded tissue samples).

Sequence identity/similarity: The identity/similarity between two or more nucleic acid sequences, or between two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods. In some embodiments, one or more disclosed peptides may comprise one or more amino acid sequences having at least 80% sequence identity (for example, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to an amino acid sequence or sequences of one or more peptides of SEQ ID NOs. 2-2273. In some embodiments, one or more disclosed nucleic acid molecules encoding one or more peptides of SEQ ID NOs. 2-2273 may comprise one or more nucleic acid sequences having at least 80% sequence identity (for example, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the corresponding one or more nucleic acid sequences o1f SEQ ID NO. 1 encoding the one or more peptides.

Sequence alignment methods for comparison and to determine sequence identity or similarity are known to those of ordinary skill in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Mo. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the internet, for use in connection with the sequence analysis programs blastp, blastn, blastx, tblastn and tblastx. Additional information can be found at the NCBI web site.

BLASTN is used to compare nucleic acid sequences, while BLASTP is used to compare amino acid sequences. If the two compared sequences share homology, then the designated output file will present those regions of homology as aligned sequences. If the two compared sequences do not share homology, then the designated output file will not present aligned sequences. vSubject: A “subject” is any multi-cellular vertebrate organism, a category that includes both human and non-human mammals (such as mice, rats, rabbits, sheep, swine, horses, cows, and non-human primates). Certain disclosed embodiments of the present invention particularly concern ungulates, even more particularly members of the family Suidae, including the genus Sus, such as Sus scrofa and Sus scrofa domesticus, and swine includes at least the genus Sus, with particular examples being Sus scrofa and Sus scrofa domesticus.

Transformed: A “transformed” cell is a cell into which has been introduced a nucleic acid molecule using molecular biology techniques known to those of ordinary skill in the art. The term encompasses all techniques by which a nucleic acid molecule might be introduced into a cell, including transfection with plasmid vectors, transformation with viral vectors, and introduction of naked DNA by lipofection, electroporation, and/or particle gun acceleration.

Vaccine: “Vaccine” refers to an immunogenic material, or a composition comprising an immunogenic material, capable of stimulating an immune response. Vaccines may be administered to prevent, ameliorate, or treat an infectious or other type of disease or diseases. The immunogenic material may include attenuated or inactivated microorganisms (such as bacteria or viruses), or antigenic proteins (including VLPs), peptides, or DNA derived from or encoding them, or combinations thereof. An attenuated vaccine is a virulent organism that has been modified to produce a less virulent form, but nevertheless retains the ability to elicit antibodies and an immune response against the virulent form. An inactivated vaccine is a previously virulent microorganism that has been killed with chemicals or heat, but which elicits antibodies against the virulent microorganism. Vaccines may elicit both prophylactic (preventative) and therapeutic responses. Methods of administration vary according to the vaccine, but may include inoculation, ingestion, inhalation, or other forms of administration. Vaccines may be administered with an adjuvant to boost the immune response.

Vector: A vector is a nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. An insertional vector is capable of inserting itself into a host nucleic acid. A vector can also include one or more selectable marker genes and other genetic elements. An expression vector is a vector that contains regulatory sequences that allow transcription and translation of an inserted gene or genes.

Virus-like particle (VLP): Virus-like particles are made up of one or more viral proteins but lack the viral genome. Because VLPs lack a viral genome, they are non-infectious.

### III. Overview of Embodiments

Immunogenic peptides associated with ASFV are disclosed, and in certain embodiments include one or more peptides of SEQ ID NOs: 2-2273, one or more constructs of SEQ ID NOs. 2310-2330, one or more domains (also referred to herein as “hotspots” as described in Example 3) of SEQ ID NOs: 2331-2335, and/or one or more full- and/or partial-length ASFV proteins (for example, one or more proteins of SEQ ID NOs: 2323-2329), or a vector or vectors comprising at least one of the peptides, constructs, domains, and/or full- and/or partial-length ASFV proteins; a cell or cells comprising at least one of the peptides, constructs, domains, and/or full- and/or partial-length ASFV proteins; or a nucleic acid construct encoding at least one of the peptides, constructs, domains, and/or full- and/or partial-length ASFV proteins. In some embodiments, disclosed compositions may include a pharmaceutically acceptable carrier, an adjuvant, an additional therapeutic, or a combination thereof.

Disclosed compositions can be formulated for administration to an animal, particularly swine, by various routes typically used to deliver a composition to an animal. In some embodiments, the composition is formulated for intranasal administration. In other embodiments, the composition is formulated for intramuscular administration.

Also provided are containers that include one or more of the compositions disclosed herein. The container may be reusable or disposable. In some embodiments, the container is a syringe. In some examples, the syringe is reusable. In other examples, the syringe is disposable. Disposable syringes generally contain a single dose of a composition. In some embodiments, the container is a vial or a bottle, such as a glass or plastic vial or bottle. In some embodiments, the vial includes a single dose of the composition. In other embodiments, the vial includes more than one dose of the composition, such as 2, 3, 4, 5, 6, 7, 8, 9, or 10 or more doses of the composition. The vial can be sterilized prior to adding the composition.

Also provided are kits that include one or more containers disclosed herein. In some embodiments, the kit comprises a bottle (such as a bottle containing a composition), a syringe, a needle, or any combination thereof. In one non-limiting example, the kit can comprise a syringe containing the composition. In another non-limiting example, the kit can comprise a syringe that is empty. A composition can be in a liquid solution or suspension, such as in PBS or water, or another acceptable carrier. A composition disclosed herein can be in a dried, tablet, and/or powdered form, such as lyophilized and/or freeze dried. Dried, powdered, and/or lyophilized forms can also be reconstituted, for example with PBS, water, an organic solvent, or another acceptable carrier. A composition can also be in a gel or syrup form. The one or more containers in the kit can include one or more additional components, such as, for example, an adjuvant, a carrier, a stabilizer, an additional therapeutic, or combinations thereof, or the additional one or more components can be in one or more separate containers in the kit. In some examples, the kits also include a device or devices that permit administration of one or more of the compositions, or of one or more of the additional components, or combinations thereof, to an animal. Examples of such devices include a syringe or syringe atomizer, such as, for example, a nasal drug delivery device, or an intramuscular drug delivery device. A kit can include (for example, in the same box or separately) a document comprising details of a composition or compositions, such as, for example, instructions for administration and/or information describing the peptides, vectors, cells, nucleic acid constructs, or combinations thereof, within the composition.

Embodiments of a method of administering one or more disclosed peptides, constructs, domains, and/or full- and/or partial-length ASFV proteins, and/or one or more nucleic acids, vectors, host cells, and/or compositions comprising the one or more peptides, constructs, domains, and/or full- and/or partial-length ASFV proteins to an animal are also disclosed. Also provided are embodiments of a method of eliciting an immune response in an animal and/or immunizing an animal against ASFV by administering to the animal a therapeutically effective amount of one or more peptides, constructs, domains, and/or full- and/or partial-length ASFV proteins, and/or one or more nucleic acids, vectors, host cells, and/or compositions comprising the one or more peptides, constructs, domains, and/or full- and/or partial-length ASFV proteins disclosed herein. In some embodiments, the composition is administered intramuscularly. In other embodiments, the composition is administered intranasally. In some embodiments, the animal is a mammal. In some embodiments, the mammal is a swine. In some embodiments, the swine is Sus scrofa domesticus.

Disclosed compositions can be used to treat (such as vaccination) adult and/or juvenile animals. Thus, in some embodiments, the animal is an adult animal. In other embodiments, the animal is a juvenile animal.

A. African Swine Fever Virus Isolates

In some embodiments, a disclosed composition comprises one or more immunogenic ASFV peptides, constructs, domains, and/or full- and/or partial-length ASFV proteins. In particular embodiments, disclosed compositions comprise one or more peptides of SEQ ID NOs. 2-2273, produced by chemical synthesis, peptide isolation, and/or recombinant methods. The native peptides of SEQ ID NOs. 2-2273 are expressed by the ASFV strain, China/2018/AnhuiXCGQ. The ASFV strain China/2018/AnhuiXCGQ genome is provided by SEQ ID NO. 1, which is incorporated by reference herein. A person of ordinary skill in the art will appreciate that the techniques disclosed herein are applicable to ASFV strains other than China/2018/AnhuiXCGQ. Other exemplary (non-limiting) ASFV strains that can be used to generate ASFV immunogenic peptides, such as peptides of SEQ ID NOs. 2-2273, are shown in Table 2. In one non-limiting example, the composition includes a viral vector expressing 1-100, or 2-100, or 1-50, or 2-50, or 2-25, or 5-25 peptides, such as 1, 2, 3, 4, 5, 6, 7, 8, 9, 10, 11, 12, 13, 14, 15, 16, 17, 18, 19, 20, 21, 22, 23, 24 or 25 peptides, selected from SEQ ID NOs 2-2273, wherein the peptides are predicted immunogenic epitopes expressed by or contained within an ASFV strain, such as the China/2018/AnhuiXCGQ ASFV strain (Accession No. MK128995.1).

A composition comprising one or more immunogenic peptides of SEQ ID NOs. 2-2273, one or more constructs of SEQ ID NOs. 2310-2330, one or more domains of SEQ ID NOs: 2331-2335, and/or one or more full- and/or partial-length ASFV proteins of SEQ ID NOs: 2323-2329 may elicit or stimulate an immune response against, or result in immunization against, one or more strains of ASFV, such as against 2, 3, 4, 5, 10, 20, or 25 strains (for example, against 1-40 ASFV strains). In one non-limiting example, a composition comprising a viral vector expressing one or more peptides selected from SEQ ID NOs. 2-2273 can be used to immunize an animal against one or more strains of ASFV, such as those listed in Table 2.

TABLE 2
Exemplary African Swine Fever Viruses

| Strain | Size (Kb) | Accession Number |
|---|---|---|
| BA71V | 170.101 | NC_001659.2/U18466.2 |
| L60 | 182.362 | KM262844.1 |
| BA71 | 180.365 | KP055815.1 |
| NHV | 172.051 | KM262845.1 |
| Kenya 1950 | 193.886 | AY261360.1 |
| Ken05/Tk1 | 191.058 | KM111294.1 |
| Ken06.Bus | 184.368 | KM111295.1 |
| 26544/OG10 | 182.906 | KM102979.1 |
| Odintsovo_02/14 | 189.333 | KP843857.1 |
| Warthog | 186.528 | AY261366.1 |
| Warmbaths | 190.773 | AY261365.1 |
| Tengani 62 | 185.689 | AY261364.1 |
| Pretorisuskop/96/4 | 190.324 | AY261363.1 |
| Mkuzi 1979 | 192.714 | AY261362.1 |
| Malawi Lil-20/1 (1983) | 187.612 | AY261361.1 |
| 47/Ss/2008 | 184.638 | KX354450.1 |
| Benin 97/1 | 182.284 | AM712239.1 |
| OURT 88/3 | 171.719 | AM712240.1 |
| E75 | 181.187 | FN557520.1 |
| Georgia 2007/1 | 189.344 | FR682468.1 |
| R8 | 188.627 | MH025916.1 |
| R7 | 188.628 | MH025917.1 |
| R25 | 188.63 | MH025918.1 |
| N10 | 188.611 | MH025919.1 |
| R35 | 188.629 | MH025920.1 |
| ASFV/POL/2015/Podlaskie | 189.394 | MH681419.1 |
| Pol16_20186_o7 | 189.401 | MG939583.1 |
| Pol16_20538_o9 | 189.399 | MG939584.1 |
| Pol16_20540_o10 | 189.405 | MG939585.1 |
| Pol16_29413_o23 | 189.393 | MG939586.1 |
| Pol17_03029_C201 | 189.405 | MG939587.1 |
| Pol17_04461_C210 | 189.401 | MG939588.1 |
| ASFV-SY18 | 189.354 | MH766894.1 |
| Kashino 04/13 | 189.387 | KJ747406.1 |
| China/2018/AnhuiXCGQ | 189.393 | MK128995.1 |
| Pig/HLJ/2018 | 189.404 | MK333180.1 |
| DB/LN/2018 | 189.404 | MK333181.1 |
| Estonia 2014 | 182.446 | LS478113.1 |
| Pol17_05838_C220 | 189.393 | MG939589.1 |

B. Nucleic Acid Molecules

Certain disclosed embodiments include one or more nucleic acid molecules that encode the amino acid sequence of one or more peptides of SEQ ID NOs. 2-2273, one or more constructs of SEQ ID NOs. 2310-2330, one or more domains of SEQ ID NOs: 2331-2335, and/or one or more full- and/or partial-length ASFV proteins of SEQ ID NOs: 2323-2329 (such as one or more nucleic acid of SEQ ID NOs. 2339-2345), or that result from the substitution of some or any of the nucleotides of one or more of the nucleic acid molecules with other nucleotides, or from the insertion or deletion of one or more of such nucleotides, provided that the resultant peptides are still suitable for inducing an immune response or ameliorating a sign or symptom of an infection, and preferably are immunogenetically equivalent to the corresponding one or more peptides, constructs, domains, and/or full- and/or partial-length ASFV proteins. Based on this information, one of ordinary skill in the art can identify the nucleic acid sequence within an ASFV genome or other ASFV nucleic acid sequence (such as, for example, a DNA, cDNA, or RNA sequence) that corresponds, for example, to the peptide of SEQ ID NO: 3, or the peptide of SEQ ID NO: 29, or the peptide of SEQ ID NO: 1092. This can be accomplished, for example, by aligning an ASFV genome, such as that of ASFV strain China/2018/AnhuiXCGQ, provided in attached SEQ ID NO: 1, and one or more of the disclosed peptide sequences, for example by using a pair-wise sequence alignment tool, such as GeneWise, provided by the European Bioinformatics Institute of the European Molecular Biology Laboratory (EMBL-EBI).

Some disclosed embodiments concern one or more isolated nucleic acid molecules, such as one or more DNA, cDNA, and/or RNA molecules. In some embodiments, a composition may comprise one or more nucleic acid molecules that encode at least one peptide of SEQ ID NOs. 2-2273, one or more constructs of SEQ ID NOs. 2310-2330, one or more domains of SEQ ID NOs: 2331-2335, and/or one or more full- and/or partial-length ASFV proteins of SEQ ID NOs: 2323-2329 (such as one or more nucleic acid of SEQ ID NOs. 2339-2345). A nucleic acid molecule disclosed herein that encodes one or more peptides, constructs, domains, and/or full- and/or partial-length ASFV proteins may also encode additional components, such as, for example, one or more multiple cloning sites, one or more expression control sequences (for example, a heterologous promoter), and/or one or more selection-related sequences, such as a nucleic acid sequence enabling selection through antibiotic resistance. In one non-limiting example, nucleic acid molecules encoding more than one, such as, for example, 2, 3, 4, 5, 6, 7, 8, 9, 10, 15, 20, or 30 peptides of SEQ ID NOs. 2-2273, are incorporated into a larger nucleic acid molecule, comprising the peptides and additional components, that is expressed by a cell and/or by a viral or bacterial vector. In another non-limiting example, nucleic acid molecules encoding one or more peptides of SEQ ID NOs. 2-2273 are incorporated into a DNA vaccine that can be administered to an animal to stimulate or elicit an immune response to one or more of the expressed peptides.

C. Peptides

Certain disclosed embodiments concern immunogenic peptides selected from SEQ ID NOs. 2-2273, constructs selected from SEQ ID NOs. 2310-2330, domains selected from SEQ ID NOs: 2331-2335, and/or full- and/or partial-length ASFV proteins selected from SEQ ID NOs: 2323-2329. Some embodiments comprise one or more peptides, constructs, domains, and/or full- and/or partial-length ASFV proteins wherein at least one amino acid of a peptide is substituted with another one or more amino acids, or an amino acid in a peptide is inserted or deleted, or combinations thereof, provided that the resultant peptide or peptides are capable of inducing an immune response or ameliorating a sign or symptom of a viral infection. Some embodiments comprise full protein, or one or more peptides of 1 to 200 amino acids, including peptides having any number of amino acids within this range, such as 5 to at least 50 amino acids in length, such as, for example, 6-40, 7-30, or 8-20 amino acids in length, with particular embodiments having from 8 to 11 amino acids.

In certain embodiments, an immunogenic composition comprises one or more peptides selected from SEQ ID NOs. 2-2273. In one non-limiting example, the peptide or peptides of a composition are synthetic and are produced chemically, using techniques well known to those of ordinary skill in the art. In another non-limiting example, the peptide or peptides of a composition are obtained from intracellular synthesis using recombinant techniques known to those of ordinary skill in the art. In other embodiments, the one or more peptides included in a composition are expressed by or contained within, or both, a nucleic acid construct, a vector or vectors, a cell or cells, or a combination thereof. In yet other embodiments, the peptides may be isolated peptides.

A disclosed immunogenic peptide or peptides may be modified, for example for the purpose of stabilizing peptide conformation, improving peptide stability against enzymatic degradation, improving peptide stability in vivo, or combinations thereof. Such modifications can include, for example, glycosylation, PEGylation, lipidation, cyclisation, acetylation, amidation, conjugation, D-amino acid incorporation, a similar modification, or combinations thereof.

The swine major histocompatibility complex (WIC), also called swine leukocyte antigen (SLA) in pigs, is associated with the porcine immune response to viral infections and vaccinations. SLA class I glycoproteins are present in all nucleated cells and present endogenous antigens that most commonly originate in the infected cell cytoplasm. The SLA class I gene cluster includes three constitutively expressed genes: SLA-1, SLA-2, and SLA-3, all of which are highly polymorphic. The different allelic forms of these genes produce proteins with binding specificities for different peptide classes. Peptides presented by SLA class I molecules on the surface of an infected cell are typically 8-11 amino acids in length. Recognition of SLA class I glycoproteins by CD8 coreceptors on cytotoxic T cells leads to destruction of the infected cell and initiates the cell-mediated immune response component of the adaptive immune response. The cell-mediated immune response, along with the humoral response (i.e., synthesis of virus-specific antibodies by B lymphocytes), leads to the production of longer-lived “memory cells” that allow for a more rapid immune response (and immunity) to subsequent infections with the same or closely-related viruses.

Newer generations of algorithms aimed at predicting high affinity immunogenic peptides no longer focus solely on binding affinity (for example to an MEW molecule, which represents a single event), and thus are less likely to yield vast lists of putative peptides that include significant numbers of false positives. The peptides disclosed herein can be, and were, generated using various bioinformatics approaches, such as, for example, predictive algorithms that can identify high density clusters of putative immunogenic peptides and/or can identify potentially immunogenic peptides based on predicted MEW binding affinity. For example, Zvi et al. (PLoS ONE 7(5):e36440, 2012; incorporated herein by reference) assessed the ability of putative immunogenic epitopes of the bacterium, Francisella tularensis, to elicit a T-cell response by, in part, mapping clusters of overlapping predicted epitopes and ranking such “hotspot” regions according to the density of the epitopes. This method complements classical binding affinity-based algorithms. Similarly, the NetMHCpan-4.0 algorithm predicts interactions of peptides with MEW class I molecules by integrating in silico-derived binding affinity information and eluted ligands derived from mass spectrometry (MS) (Jurtz, et al. J. Immunol 199(9):3360-3368, 2017; incorporated herein by reference). This approach incorporates the increasing availability of MS-derived information about peptide-processing steps in the MEW class I presentation pathway and the length distributions of presented peptides to reduce the number of false positive hits that are typically generated from in silico-derived binding affinity information alone.

Immunogenicity of the disclosed peptides can be validated using various methods for measuring an immune response in vitro or in vivo. Such methods are well known to those of ordinary skill in the art, and the present invention is not limited to using specific assays. In one non-limiting example, relevant peptides can be synthesized and then screened against peripheral blood lymphocytes or spleen-derived cells using enzyme-linked immunosorbent spot (ELISpot) assays. In another non-limiting example, animals can be administered varying concentrations of a given composition or compositions, one or more times, at one or more different time intervals, and the presence of anti-peptide antibodies in treated versus non-treated animal serum can be established using enzyme-linked immune absorbent assays (ELISAs). In another non-limiting example, animals can be administered varying concentrations of a given composition or compositions, one or more times, at one or more different time intervals, challenged with an ASFV strain, and observed over time for ASF symptom development.

D. Constructs

In some embodiments, the one or more peptides of SEQ ID NOs. 2-2273, one or more

domains of SEQ ID NOs: 2331-2335 (also referred to as “hotspots,” see Example 3), and/or one or more full- and/or partial-length ASFV proteins (for example, one or more proteins of SEQ ID NOs. 2323-2329) is/are incorporated into a larger amino acid construct. Exemplary constructs are provided as SEQ ID NOs. 2310-2330. Such constructs can further comprise, for example, an N-terminal HLT, Sumo, and/or MBP fusion protein. Such constructs can comprise an N-terminal His-tag. For example, if a construct comprises a HLT, Sumo, and/or MBP fusion protein, the His-tag can be appended to the N-terminus of the fusion protein.

In some embodiments, one or more peptides of SEQ ID NOs. 2-2273, one or more domains of SEQ ID NOs: 2331-2335, and/or one or more full- and/or partial-length ASFV proteins (for example, one or more proteins of SEQ ID NOs. 2323-2329) included in one or more constructs may further comprise one or more spacer sequences (such as GPGPG and/or AAY) between all or some (such as 1, 2, 3, 4, 5, 6, 7, 8, 9, 10, 15, 20, or 25) of the sequences encoding the one or more peptides, domains, and/or full- and/or partial-length ASFV proteins. Additional spacer sequences that can be used in a construct disclosed herein are known to those of ordinary skill in the art, and the present disclosure is not limited to the particular spacer sequences disclosed herein.

In some embodiments, a construct comprises one or more nucleotide sequences encoding one or more detection sequences and, optionally, a linker (such as GSSG). A linker and detection sequence can be located, for example, at the C-terminus of the sequences encoding the one or more peptides, domains, full- and/or partial-length ASFV proteins, and/or spacer sequences, such that the linker is located between the C-terminus of the construct and the N-terminus of the detection sequence. Linkers and detection sequences and methods of, for example, tagging an expressed sequence for detection of a protein in a host cell, in a lysate, in a supernatant, in a subject, and/or in a sample obtained from a subject, are known to those of ordinary skill in the art, and the present disclosure is not limited to one or any specific detection sequence or to one or any specific linker sequence. An exemplary detection sequence is the HiBiT (Promega) sequence GSGWRLFKKLS, (or GSSGGSGWRLFKKLS with the optional exemplary linker) useful for detection of, for example, a protein product that results from expression of one or more nucleic acid molecules, such as in a lysate and/or supernatant collected from a host cell culture, such as from a host cell culture comprising E. coli cells transformed with the one or more nucleic acid molecules. Another exemplary detection sequence is a sequence encoding a histidine tag (His-tag) (such as a nucleotide sequence encoding the amino acid sequence HHHHHH, wherein each H is encoded by a CAC or CAT codon) useful for detection of, for example, a protein product that results from expression of one or more nucleic acid molecules, such as in a lysate and/or supernatant collected from a host cell culture, such as from a host cell culture comprising E. coli cells transformed with the one or more nucleic acid molecules.

E. Vectors and Host Cells

Multiple types and versions of vectors, nucleic acid molecules, and cells comprising one or more peptides of SEQ ID NOs. 2-2273, one or more constructs of SEQ ID NOs. 2310-2330, one or more domains of SEQ ID NOs: 2331-2335, and/or one or more full- and/or partial-length ASFV proteins (for example, one or more proteins of SEQ ID NOs. 2323-2329 and/or nucleic acids of SEQ ID NOs. 2339-2345) are within the scope of the present invention. Methods of producing the vectors, nucleic acid molecules, and cells are known to those of ordinary skill in the art, and the present disclosure is not limited to using one or more specific vector, nucleic acid molecule, or host cell production methods, or to specific vectors, nucleic acid molecules, or cell types. Generally, vectors and host cells that include or produce one or more peptides of SEQ ID NOs. 2-2273 comprise one or more nucleic acid molecules encoding one or more peptides of SEQ ID NOs. 2-2273 (such as one or more nucleic acid molecules of SEQ ID NOs. 2286-2309), and are typically generated to express the peptides. Naked nucleic acid molecules, such as, for example, a plasmid generated for use in a DNA vaccine, are typically produced to express one or more peptides of SEQ ID NOs. 2-2273 following cellular transformation with the nucleic acid molecules. Thus, one or more compositions comprising at least one vector, nucleic acid molecule, or host cell described herein, or combinations thereof, can be administered to an animal to, for example, produce an immune response against ASFV, and/or to immunize an animal against ASFV, or to ameliorate or eliminate one or more symptoms associated with ASF.

In some embodiments, the one or more nucleic acid molecules encoding one or more peptides of SEQ ID NOs. 2-2273, one or more domains of SEQ ID NOs: 2331-2335, and/or one or more full- and/or partial-length ASFV proteins (for example, one or more proteins of SEQ ID NOs. 2323-2329 and/or nucleic acids of SEQ ID NOs. 2339-2345) is incorporated into a larger nucleic acid construct for measuring expression of the one or more nucleic acid molecules, for example, in a host cell. Exemplary constructs are provided as SEQ ID NOs. 2310-2330. Such constructs can further comprise, for example, one or more plasmid vectors such as a pHLT, pSumo, and/or pMBP plasmid, for example to append an N-terminal HLT, Sumo, and/or MBP fusion protein. Such constructs can comprise an N-terminal His-tag. For example, if a construct comprises a HLT, Sumo, and/or MBP fusion protein, the His-tag can be appended to the N-terminus of the fusion protein.

In some embodiments, nucleic acid molecules encoding one or more peptides of SEQ ID NOs. 2-2273, one or more domains of SEQ ID NOs: 2331-2335, and/or one or more full- and/or partial-length ASFV proteins (for example, one or more proteins of SEQ ID NOs. 2323-2329 and/or nucleic acids of SEQ ID NOs. 2339-2345) included in one or more constructs further comprise one or more spacer sequences (such as GPGPG and/or AAY) between all or some (such as 1, 2, 3, 4, 5, 6, 7, 8, 9, 10, 15, 20, or 25) of the nucleotide sequences encoding the one or more peptides, domains, and/or full- and/or partial-length ASFV proteins. Additional spacer sequences that can be used in a nucleic acid molecule disclosed herein are known to those of ordinary skill in the art, and the present disclosure is not limited to the particular spacer sequences disclosed herein.

In some embodiments, a construct comprises one or more nucleotide sequences encoding one or more detection sequences and, optionally, a linker (such as GSSG). A linker and detection sequence can be located, for example, at the C-terminus of the nucleotide sequences encoding the one or more peptides, domains, full- and/or partial-length ASFV proteins, and/or spacer sequences, such that the linker is located between the C-terminus of the construct and the N-terminus of the detection sequence. Linkers and detection sequences and methods of, for example, tagging an expressed sequence for detection of a nucleic acid molecule or protein in a host cell, in a lysate, in a supernatant, in a subject, and/or in a sample obtained from a subject, are known to those of ordinary skill in the art, and the present disclosure is not limited to one or any specific detection sequence or to one or any specific linker sequence. An exemplary detection sequence is a nucleic acid molecule encoding the HiBiT (Promega) sequence GSGWRLFKKLS, (or GSSGGSGWRLFKKLS with the optional exemplary linker) useful for detection of, for example, a protein product that results from expression of one or more nucleic acid molecules, such as in a lysate and/or supernatant collected from a host cell culture, such as from a host cell culture comprising E. coli cells transformed with the one or more nucleic acid molecules. Another exemplary detection sequence is a sequence encoding a histidine tag (His-tag) (such as a nucleotide sequence encoding the amino acid sequence HHHHHH, wherein each H is encoded by a CAC or CAT codon) useful for detection of, for example, a protein product that results from expression of one or more nucleic acid molecules, such as in a lysate and/or supernatant collected from a host cell culture, such as from a host cell culture comprising E. coli cells transformed with the one or more nucleic acid molecules.

In some embodiments, one or more nucleic acid molecules encoding one or more peptides of SEQ ID NOs. 2-2273, one or more constructs of SEQ ID NOs. 2310-2330, one or more domains of SEQ ID NOs: 2331-2335, and/or one or more full- and/or partial-length ASFV proteins (for example, one or more proteins of SEQ ID NOs. 2323-2329 and/or nucleic acids of SEQ ID NOs. 2339-2345) is/are incorporated into a larger nucleic acid construct, such as a plasmid, for example, for direct introduction into an animal. Such a nucleic acid construct can be introduced into an animal by any suitable technique, such as through saline injection, particle gun acceleration, any suitable known or hereafter discovered method for administering a DNA or RNA vaccine to a subject, or combinations thereof, and such methods are known or will be understood by those of ordinary skill in the art. In one non-limiting example, one or more nucleic acid molecules encoding one or more, such as, for example, 1, 2, 3, 4, 5, 8, 10, 15, or 20, peptides of SEQ ID NOs. 2-2273, is incorporated into a plasmid, and a composition comprising the plasmid is administered to swine.

In some embodiments, a nucleic acid molecule or nucleic acid molecules encoding one or more peptides of SEQ ID NOs. 2-2273, one or more constructs of SEQ ID NOs. 2310-2330, one or more domains of SEQ ID NOs: 2331-2335, and/or one or more full- and/or partial-length ASFV proteins (for example, one or more proteins of SEQ ID NOs. 2323-2329 and/or nucleic acids of SEQ ID NOs. 2339-2345) may be incorporated into a viral vector. In some embodiments, the viral vector can be a herpesvirus, Adenovirus, Circovirus, Alphavirus, Orthopoxvirus, Avulavirus, Suipoxvirus, or any combination thereof. In one non-limiting example, the viral vector is a Pseudorabies virus, Porcine circovirus, Sindbis virus, Vaccinia virus, Newcastle virus, or Swinepox virus. In one specific non-limiting example, a nucleic acid molecule encoding one or more, such as, for example, 1, 2, 3, 4, 5, 8, 10, 15, or 20, peptides of SEQ ID NOs. 2-2273 is incorporated into a Vaccinia viral vector, and a composition comprising the vector is administered to swine.

In other embodiments, a nucleic acid molecule or nucleic acid molecules encoding one or more peptides of SEQ ID NOs. 2-2273, one or more constructs of SEQ ID NOs. 2310-2330, one or more domains of SEQ ID NOs: 2331-2335, and/or one or more full- and/or partial-length ASFV proteins (for example, one or more proteins of SEQ ID NOs. 2323-2329 and/or nucleic acids of SEQ ID NOs. 2339-2345) may be incorporated into a host cell. In one non-limiting example, the host cell is a recombinant yeast, such as, for example, a yeast of genus Pichia or genus Saccharomyces. In specific non-limiting examples, the recombinant yeast is Pichia pastoris or Saccharomyces cerevisiae. In another non-limiting example, the host cell is a recombinant prokaryote, such as, for example, a bacterium of genus Salmonella, Escherichia, Listeria, Shigella, Pseudomonas, Bordetella, Bacillus, Yersinia, Mycobacterium, Lactobacillus, Lactococcus, or Vibrio. In specific non-limiting examples, the recombinant bacterium is Salmonella enterica, Escherichia coli, Listeria monocytogenes, Shigella flexneri, Pseudomonas aeruginosa, Bacillus subtilis, Yersinia enterocolitica, Mycobacterium smegmatis, Mycobacterium bovis, Lactococcus lactis, or Vibrio anguillarum. One or more of the nucleic acid molecules can be incorporated into a host cell by one of several techniques by which a nucleic acid molecule might be introduced into a cell. Techniques, such as, for example, transformation with a plasmid encoding one or more peptides of SEQ ID NOs. 2-2273, are commonly known to a person of ordinary skill in the art. In one specific non-limiting example, a plasmid encoding one or more, such as, for example, 1, 2, 3, 4, 5, 8, 10, 15, or 20, peptides of SEQ ID NOs. 2-2273, is incorporated into a Saccharomyces cerevisiae host cell, and a composition comprising the transformed host cell is administered to swine. In another specific non-limiting example, a plasmid encoding one or more, such as, for example, 1, 2, 3, 4, 5, 8, 10, 15, or 20, peptides of SEQ ID NOs. 2-2273, is incorporated into a Salmonella enterica host cell, and a composition comprising the transformed host cell is administered to swine.

### IV. Composition

Disclosed herein are compositions comprising one or more immunogenic peptides of SEQ ID NOs. 2-2273, one or more constructs of SEQ ID NOs. 2310-2330, one or more domains of SEQ ID NOs: 2331-2335, and/or one or more full- and/or partial-length ASFV proteins (for example, one or more proteins of SEQ ID NOs. 2323-2329 and/or nucleic acids of SEQ ID NOs. 2339-2345), and/or comprising one or more vectors and/or cells and/or nucleic acid molecules comprising or encoding one or more of the peptides, constructs, domains, and/or full- and/or partial-length ASFV proteins. Disclosed compositions may be administered to an animal, particularly swine. One or more of the compositions can be used, for example, to elicit an immune response against ASFV, to immunize a subject against ASFV, to ameliorate and/or eliminate one or more symptoms associated with ASF, and/or to mitigate a future outbreak by serving as a pre-outbreak vaccine.

In some embodiments, the composition includes one or more peptides of SEQ ID NOs. 2-2273, one or more constructs of SEQ ID NOs. 2310-2330, one or more domains of SEQ ID NOs: 2331-2335, and/or one or more full- and/or partial-length ASFV proteins (for example, one or more proteins of SEQ ID NOs. 2323-2329 and/or nucleic acids of SEQ ID NOs. 2339-2345). In other embodiments, the composition includes a vector or vectors, such as, for example, a viral or bacterial vector, comprising the disclosed one or more peptides, constructs, domains, and/or full- or partial-length ASFV proteins. In one non-limiting example, the viral vector is a pseudorabies virus. In another non-limiting example, the viral vector is a modified vaccinia Ankara virus. In other embodiments, the composition includes a DNA plasmid and/or other nucleic acid construct encoding one or more peptides, constructs, domains, and/or full- or partial-length ASFV proteins. In another embodiment, the invention relates to one or more peptides of SEQ ID NOs. 2-2273, obtainable through expression of a nucleic acid construct and/or other encoding sequence. In another embodiment, the invention relates to a cell and/or vector containing a gene construct encoding the disclosed peptides. In one non-limiting example, one or more peptides of SEQ ID NOs. 2-2273, one or more constructs (for example, one or more amino acid sequences of SEQ ID NOs. 2310-2330), one or more domains (also referred to herein as “hotspots” as described in Example 3; for example, one or more amino acid sequences of SEQ ID NOs: 2331-2335), and/or one or more full- and/or partial-length ASFV proteins (for example, one or more proteins of SEQ ID NOs: 2323-2329 and/or nucleic acids of SEQ ID NOs. 2339-2345) is expressed in a cell and/or by one or more vectors. Thus, the process of expressing and/or producing peptides according to SEQ ID NOs. 2-2273 constitutes an additional aspect of this invention. Such peptides can also be produced synthetically as commonly understood by one of ordinary skill in the art to which this disclosure belongs. In one non-limiting example, the composition includes chemically synthesized peptides or peptides obtained from intracellular synthesis using recombinant techniques well known to those of ordinary skill in the art.

Disclosed immunogenic compositions may include other agents. Some embodiments concern a pharmaceutical composition comprising a therapeutically effective amount of a DNA or RNA construct comprising one or more peptides of SEQ ID NOs. 2-2273, one or more constructs of SEQ ID NOs. 2310-2330, one or more domains of SEQ ID NOs: 2331-2335, and/or one or more full- and/or partial-length ASFV proteins (for example, one or more proteins of SEQ ID NOs. 2323-2329 and/or nucleic acids of SEQ ID NOs. 2339-2345) or of a vector comprising one or more of the peptides, domains, and/or ASFV proteins, or of a cell comprising one or more of the peptides, domains, and/or ASFV proteins, together with one or more additional components. In non-limiting examples, the additional components are an appropriate carrier, such as, for example, PBS, and an appropriate adjuvant. In some embodiments, the peptides, nucleic acid constructs, vectors, and/or cells are present in an acceptable carrier such as saline, buffered saline, dextrose, water, glycerol, oil, ethanol, or combinations thereof. The carrier or composition containing the carrier, or both, can be sterile. The composition can also comprise suitable amounts of pH buffering agents, or wetting or emulsifying agents. The composition can also comprise conventional pharmaceutical materials such as, for example, acceptable buffers, preservatives, salts to adjust osmotic pressure, and similar. The composition can also contain adjuvant materials, such as, for example, oil adjuvants, oil-in-water adjuvants, water-in-oil adjuvants, water-in-oil-in-water adjuvants, aluminum hydroxide, potassium hydroxide, complete Freund's adjuvant, incomplete Freund's adjuvant, saponine, squalene, immune-stimulating complexes (ISCOMs), liposomes, polysaccharides, derivatized polysaccharides, oligonucleotides, cytokines, bacterial derivatives, viral derivatives, gel adjuvants, such as, for example, Emulsigen-D, or carbomer-based adjuvants, such as, for example, Carbigen. The composition can include one or more peptides of SEQ ID NOs. 2-2273 combined with one or more adjuvants by chemical conjugation, such as, for example, through oxime ligation, native chemical ligation, thioether ligation, hydrazine ligation between an aldehyde group and hydrazine (NH₂NH—) group, maleimide-thiol group reaction, CuAAC reaction, or similar. The composition can include one or more peptides of SEQ ID NOs. 2-2273, combined by polymerization using one or more chemical methods, recombinant techniques, and/or enzymatic reactions. Disclosed compositions can also include one or more peptides of SEQ ID NOs. 2-2273, having undergone modifications, such as, for example, glycosylation, PEGylation, lipidation, cyclisation, acetylation, amidation, conjugation, D-amino acid incorporation, or a similar modification, or combinations thereof. The composition can be a liquid solution or suspension, syrup, emulsion, microemulsion, aerosol, tablet, pill, capsule, gel, sustained release formulation, or powder. In one non-limiting example, the composition is a lyophilized or freeze-dried powder, or a liquid. The composition can be formulated as a suppository, with traditional binders and carriers such as triglycerides. Oral formulations can include standard carriers such as, for example, starch, mannitol, sodium saccharine, lactose, cellulose, magnesium stearate, magnesium carbonate, or combinations thereof. Mucosal formulations can include mucoadhesive polymers, such as, for example, chitosan. Disclosed compositions can also include one or more additional therapeutics, such as, for example, other vaccines, including, but not limited to, subunit vaccines, live attenuated virus vaccines, DNA vaccines, RNAi vaccines, inactivated vaccines, bacterial vaccines, yeast vaccines, or combinations thereof. Such vaccines may also include, for example, porcine reproductive and respiratory syndrome virus vaccines, porcine circovirus-2 vaccines, immunocastration vaccines, other specific vaccines, or combinations thereof. Other therapeutics can also include compounds or compositions aimed at reducing or alleviating the symptoms of ASF, such as, for example, anti-inflammatories, anti-diarrheals, appetite stimulants, anti-nausea medications, respiratory therapeutics, iron dextran, or combinations thereof.

### V. Methods of Stimulating and Measuring an Immune Response

The disclosed invention also concerns embodiments of a method of using the disclosed compositions. For example, one embodiment comprises providing at least one peptide, vector, nucleic acid molecule, and/or composition described herein, and administering an effective amount thereof to an animal, such as swine. One non-limiting example of a method according to the present disclosure includes eliciting or stimulating an immune response in an animal to one or more peptides of SEQ ID NOs. 2-2273, one or more constructs of SEQ ID NOs. 2310-2330, one or more domains of SEQ ID NOs: 2331-2335, and/or one or more full- and/or partial-length ASFV proteins (for example, one or more proteins of SEQ ID NOs. 2323-2329 and/or nucleic acids of SEQ ID NOs. 2339-2345). In another non-limiting example, the method includes vaccinating or immunizing an animal against ASFV using a composition comprising a viral vector expressing one or more peptides of SEQ ID NOs. 2-2273, one or more constructs of SEQ ID NOs. 2310-2330, one or more domains of SEQ ID NOs: 2331-2335, and/or one or more full- and/or partial-length ASFV proteins (for example, one or more proteins of SEQ ID NOs. 2323-2329 and/or nucleic acids of SEQ ID NOs. 2339-2345). In some embodiments, the composition is administered using any suitable route of administration, such as, for example, intramuscular or intranasal administration. Examples of animals that can be administered at least one of the disclosed compositions include animals that can be (or are) infected with ASFV. Examples of such animals include but are not limited to, mammalian subjects, ungulates, such as swine, such as, for example, a sow during pregnancy. An animal administered a composition can be an adult or a juvenile.

Disclosed compositions can be used to stimulate or elicit an immune response to ASFV in an animal. In some examples, the method comprises administering a therapeutically effective amount of a composition comprising one or more peptides of SEQ ID NOs. 2-2273, one or more constructs of SEQ ID NOs. 2310-2330, one or more domains of SEQ ID NOs: 2331-2335, and/or one or more full- and/or partial-length ASFV proteins (for example, one or more proteins of SEQ ID NOs. 2323-2329 and/or nucleic acids of SEQ ID NOs. 2339-2345) to an animal, particularly swine, to elicit an immune response to ASFV in the animal. Methods of determining whether an immune response has been elicited or stimulated are known to those of ordinary skill in the art. In some examples, an immune response is achieved if there is an observed reduction in illness (such as reduction in symptoms), reduction in viral titers, reduction in mortality rate, or a combination thereof. In some examples, the disclosed method reduces symptoms of ASFV infection in an animal administered a composition entirely, or by at least 10%, at least 20%, at least 30%, at least 40%, or at least 50%, at least 60%, at least 70%, at least 80%, or at least 90%, for example as compared to an equivalent animal not administered the composition. In some examples, the disclosed composition or method, or both, reduces viral titer in an animal administered a composition, such as by at least 10% to at least 100%, 20% to at least 100%, 30% to at least 100%, 40% to at least 100%, 50% to at least 100%, 60% to at least 100%, 70% to at least 100%, 80% to at least 100%, 90% to at least 100%, at least 2-fold, at least 3-fold, at least 4-fold, or at least 5-fold, for example as compared to an equivalent animal not administered the composition. In some examples, the disclosed method increases survival following subsequent viral challenge in animals administered a composition by at least 10%, at least 20%, at least 30%, at least 40%, at least 50%, at least 60%, at least 70%, at least 80%, at least 90%, or at least 95%, for example as compared to an equivalent animal not administered the composition.

In some examples, the method includes administering a therapeutically effective amount of a composition comprising a viral vector expressing one or more peptides of SEQ ID NOs. 2-2273, one or more constructs of SEQ ID NOs. 2310-2330, one or more domains of SEQ ID NOs: 2331-2335, and/or one or more full- and/or partial-length ASFV proteins (for example, one or more proteins of SEQ ID NOs. 2323-2329 and/or nucleic acids of SEQ ID NOs. 2339-2345), thereby immunizing the animal against ASFV. In some examples, an immune response is achieved if there is an observed reduction in illness (such as reduction in symptoms), reduction in viral titers, protection from death, or a combination thereof.

In some embodiments, an animal can be administered (such as intramuscularly) a therapeutically effective amount of about 1 to about 100 μg of each of at least one peptide of SEQ ID NOs. 2-2273, one or more constructs of SEQ ID NOs. 2310-2330, one or more domains of SEQ ID NOs: 2331-2335, and/or one or more full- and/or partial-length ASFV proteins (for example, one or more proteins of SEQ ID NOs. 2323-2329 and/or nucleic acids of SEQ ID NOs. 2339-2345). In some embodiments, an animal can be administered (such as intramuscularly) a therapeutically effective amount of about 10³ to about 10⁹ CCID⁵⁰, such as about 10⁶ CCID⁵⁰, of each of at least one viral vector, for example, a pseudorabies virus or a modified vaccinia Ankara virus, expressing one or more peptides of SEQ ID NOs. 2-2273, one or more constructs of SEQ ID NOs. 2310-2330, one or more domains of SEQ ID NOs: 2331-2335, and/or one or more full- and/or partial-length ASFV proteins (for example, one or more proteins of SEQ ID NOs. 2323-2329 and/or nucleic acids of SEQ ID NOs. 2339-2345). However, a person of ordinary skill in the art is capable of determining a therapeutically effective amount (for example, an amount that provides protection against ASFV infection) of, for example, one or more peptides of SEQ ID NOs. 2-2273, one or more constructs of SEQ ID NOs. 2310-2330, one or more domains of SEQ ID NOs: 2331-2335, and/or one or more full- and/or partial-length ASFV proteins (for example, one or more proteins of SEQ ID NOs. 2323-2329 and/or nucleic acids of SEQ ID NOs. 2339-2345), or of a viral vector expressing one or more of the peptides, domains, and/or ASFV proteins, to administer to an animal.

Methods for determining whether a composition disclosed herein can (or did) stimulate or elicit an immune response, such as achieve successful immune protection, are known to those of ordinary skill in the art, and the disclosure is not limited to the use of specific assays. Following administration of a composition provided herein, one or more assays may be performed to assess the resulting immune response. In one non-limiting example, one or more assays are also performed prior to administration of the composition to provide a baseline or control. Samples, such as a blood, serum, and/or peripheral blood macrophage cell (PBMC) sample, can be collected from an animal following administration of a composition. In some examples, a sample, or samples, is collected at least 1 week, at least 2 weeks, at least 3 weeks, at least 4 weeks, at least 5 weeks, at least 8 weeks, or at least 10 weeks (such as 1, 2, 3, 4, 5, 6, 7, 8, 9, 10, 11, or 12 weeks) after the first administration. Additional samples can also be obtained, for example following subsequent administrations of the same or different composition or compositions.

### VI. Methods of Administration

Embodiments of peptides, immunogenic compositions, vectors, cells, and/or nucleic acid constructs can be administered to an animal by any of the routes typically used for introducing a pharmaceutical composition or compositions into an animal. Methods of administration include, but are not limited to, intramuscular, oral, intravenous, intradermal, intraperitoneal, subcutaneous, parenteral, mucosal, rectal, vaginal, inhalation, intranasal, or combinations thereof. Parenteral administration, such as, for example, intramuscular, intravenous, or subcutaneous administration, is commonly achieved by injection. Administration can be local or systemic, or combinations thereof. Injectables can be prepared, for example, as emulsions, as solid forms suitable for solution or suspension in liquid prior to injection, or as liquid suspensions or solutions. Injection suspensions or solutions can be prepared from sterile powders, tablets, granules, or similar, or combinations thereof.

The composition or compositions administered to an animal may be administered with at least one acceptable carrier. Acceptable carriers are determined in part by the particular composition being administered, as well as by the particular method used to administer the composition. Thus, there is a wide variety of acceptable formulations of compositions of the present disclosure.

Preparations for parenteral administration include sterile aqueous or non-aqueous solutions, suspensions, and/or emulsions, such as, for example, oil-in-water and/or water-in-oil emulsions. Preparations for parenteral administration can also include adjuvants and/or polymers, such as, for example, CpG oligodeoxynucleotides (CpG ODN), Carbigen, Polygen, ISA 201 or 206 (such as Montanide ISA 201 VG), Quil-A, trehalose-6,6-dibehenate (TBD), toll-like receptor (TLR) ligand-based adjuvants (such as TLR7/8 adjuvants, such as R848 (Resiquimod)), cyclic diguanylate monophosphate (c-di-GMP), polyinosinic-polycytidylic acid (poly (I:C)), or combinations thereof. Examples of non-aqueous solvents are alcohols or glycols, such as propylene glycol, polyethylene glycol, vegetable oils such as olive oil, and injectable organic esters such as ethyl oleate. Aqueous carriers include water, alcoholic/aqueous solutions, emulsions, or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and similar. Preservatives and other additives may also be present, such as, for example, antimicrobials, antioxidants, chelating agents, inert gases and similar.

In some examples, disclosed embodiments are formulated for mucosal vaccination, such as oral, intranasal, pulmonary, rectal, and vaginal. In one non-limiting example, this is achieved by intranasal administration. For example, the disclosed compositions can include one or more biodegradable, polymeric carriers that interact with one or more mucosal membranes. Polymers such as polylactide-co-glycolide (PLGA), chitosan (for example in the form of chitosan nanoparticles, such as N-trimethyl chitosan (TMC)-based nanoparticles), alginate (such as sodium alginate), carbopol, and carbopol-based polymers can be included. The composition can include one or more hydrophilic polymers, such as sodium alginate or carbopol, for example in combination with starch. The composition can be formulated as a particulate delivery system used for nasal administration. Thus, the composition can include liposomes, immune-stimulating complexes (ISCOMs) and/or polymeric particles, such as virosomes. The compositions can also include one or more lipopeptides of bacterial origin, or their synthetic derivatives, such as Pam3Cys, (Pam2Cys, single/multiple-chain palmitic acids and lipoamino acids (LAAs). The compositions can also include one or more adjuvants, such as, for example, one or more of CpG oligodeoxynucleotides (CpG ODN), Flt3 ligand, Carbigen, c-di-GMP, poly (I:C), and monophosphoryl lipid A (MLA).

Compositions disclosed herein may be administered to maternally-derived antibody (MDA) positive animals. If a given vaccine stimulates a humoral immune response, sows may transfer MDAs to piglets, and this may delay the opportunity to vaccinate piglets. However, a T cell epitope vaccine may not be delayed by MDA.

A. Timing of Administration

Disclosed compositions may be administered as a single dose or as multiple doses (for example, boosters). In some examples, a first administration is followed by a second administration. For example, the second administration can be with the same, or with a different composition than the first composition administered. In one specific non-limiting example, the second administration is with the same composition as the first composition administered. In another specific non-liming example, the second administration is with a different composition than the first composition administered. For example, if a first composition comprised 10 peptides selected from SEQ ID NOs: 2-2273, the second composition could include 20 different peptides selected from SEQ ID NOs: 2-2273, wherein all 30 peptides are different. In some examples, an animal is administered one or more compositions comprising a viral vector expressing one or more peptides of SEQ ID NOs. 2-2273, one or more constructs of SEQ ID NOs. 2310-2330, one or more domains of SEQ ID NOs: 2331-2335, and/or one or more full- and/or partial-length ASFV proteins (for example, one or more proteins of SEQ ID NOs. 2323-2329 and/or nucleic acids of SEQ ID NOs. 2339-2345), and is subsequently administered one or more vaccines comprising a live attenuated ASFV.

In some examples, a composition or compositions is administered in multiple doses, such as 2, 3, 4, 5, 6, 7, 8, 9, or 10 doses (such as 2-4 doses). In these examples, the timing between the doses can be at least 1 week, at least 2 weeks, at least 3 weeks, at least 4 weeks, at least 6 weeks, at least 8 weeks, at least 12 weeks, at least 2 months, at least 3 months, at least 4 months, at least 5 months, at least 6 months, at least 1 year, at least 2 years, at least 5 years, or at least 10 years, such as 1-4 weeks, 2-3 weeks, 1-6 months, 2-4 months, 1-10 years, or 2-5 years, or combinations thereof. In one non-limiting example, wherein there are at least three administrations, the timing between the first and second, and second and third doses, can be the same or different.

B. Dosages

The dose administered to a subject in the context of the present disclosure should be sufficient to induce a beneficial therapeutic response in a subject over time, or to inhibit ASFV infection. The dose can vary from subject-to-subject depending on the species, age, weight, and general condition of the subject, the severity of the infection being treated, whether the dose is being used to treat, alleviate, or inoculate against an infection, the particular composition being used, and/or the mode of administration. An appropriate dose can be determined by one of ordinary skill in the art using routine experimentation.

In some embodiments, the animal is administered (for example, intramuscularly) about 0.1 to about 100 μg of a given peptide in the composition, such as about 1 μg to about 5 μg, about 1 μg to about 50 μg, about 1 μg to about 25 μg, about 5 μg to about 20 μg, or about 10 μg to about 15 μg of each of the at least one peptide in the composition. In one specific non-limiting example, the subject is administered (for example, intramuscularly) about 10 μg, about 15 μg, about 20 μg, or about 30 μg of each of at least two different peptides. In one non-limiting example, the animal is administered one composition at a first administration amount, a second composition at a second administration amount, and a third composition at a third administration amount. Moreover, the composition or compositions at each administration may be the same or different.

In some embodiments, the animal is administered (for example, intramuscularly) about 10² to about 10⁹ CCID⁵⁰ of a pseudorabies viral vector or a modified vaccinia Ankara viral vector expressing at least one peptide of SEQ ID NOs. 2-2273, one or more constructs of SEQ ID NOs. 2310-2330, one or more domains of SEQ ID NOs: 2331-2335, and/or one or more full- and/or partial-length ASFV proteins (for example, one or more proteins of SEQ ID NOs. 2323-2329 and/or nucleic acids of SEQ ID NOs. 2339-2345), such as about 10³ to about 10⁵, about 10⁴ to about 10⁶, about 10⁵ to about 10⁷, about 10⁶ to about 10⁸, or about 10⁷ to about 10⁹, of the viral vector within a single dose. In one specific non-limiting example, the subject is administered (for example, intramuscularly) about 10⁴, about 10⁵, about 10⁶, or about 10⁷ CCID⁵⁰ of each of at least two viral vectors expressing the same or different one or more peptides of SEQ ID NOs. 2-2273, one or more constructs of SEQ ID NOs. 2310-2330, one or more domains of SEQ ID NOs: 2331-2335, and/or one or more full- and/or partial-length ASFV proteins (for example, one or more proteins of SEQ ID NOs. 2323-2329 and/or nucleic acids of SEQ ID NOs. 2339-2345). In another non-limiting example, the animal is administered one viral vector at a first administration amount, a second viral vector at a second administration amount, and a third viral vector at a third administration amount.

### VII. Cinnamon Extract Adjuvants

Adjuvants included in some embodiments of compositions disclosed herein can include a cinnamon-derived product, such as cinnamon oil (See U.S. Patent No. 2006/0275515, “Antiviral preparations obtained from a natural cinnamon extract,” which is incorporated by reference herein). Certain cinnamon-derived adjuvants concern a composition produced by extraction, or by fractionating an extraction composition. Particular embodiments concern an aqueous extract of cinnamon bark (Cinnamomum sp.), but other polar solvents, such as, for example, alcohols and glycols, also may be used. One or more compounds in the extract, or in a fraction of the extract, may be processed to form a precipitate. For example, active antiviral fractions of the extract may have an absorbance at 280 nm of between 15 and 20 O.D., and/or may comprise one or more substances having a molecular weight greater than 10 kDa, such as at about 15 O.D. In one preferred embodiment, an isolated active fraction of cinnamon bark having antiviral activity has in addition one or more of the following chemical properties:
1. It is precipitated by various chloride salts such as KCl, NaCl, MgCl₂, SrCl₂, CuCl₂, or ZnCl₂.
2. It exhibits absorbance at 280 nm of 15 O.D/mg. cm.
3. It maintains most of its activity after incubation in 0.1M NaOH, or 0.1M HCl, or 0.1M H₂SO₄.
4. It can be extracted into an aqueous solution, or into an organic solution, such as an alcoholic solvent or acetone in a relatively inexpensive and simple manner.
5. It can be maintained for a long period of time (at least two years) as a stable powder or in solution in a refrigerator or at room temperature;
6. It is heat-stable and can thus be sterilized at temperature up to at least 134° C.

Useful extraction compositions may be made by any suitable process. One suitable embodiment comprises forming a cinnamon bark powder, and forming a solution or suspension comprising the cinnamon bark powder. The process can involve forming an appropriate solution using either an aqueous solvent or an organic solvent. Certain embodiments concern forming an aqueous solution, and the solution may then be centrifuged and a supernatant collected that includes an antiviral active fraction. A precipitate may also be formed, such as by evaporation or by adding a precipitation aid, such as a salt, and more particularly a chloride salt, such as KCl, NaCl, MgCl₂, SrCl₂, CuCl₂, ZnCl₂ or combinations thereof.

The precipitate may be further fractionated or purified. One such process is a chromatographic process. For example, the precipitate may be dissolved in water at a pH of about 7. The solution can be added to a Sepharose column and eluted with a buffer and a saccharide. A more specific process comprises using a 0.02 M aqueous phosphate buffer at a pH of 7.0 to form a solution, forming a precipitate by adding 0.15 M KCl or 0.08 M MgCl₂, dissolving the precipitate in water or 0.01 M phosphate buffer at pH 7.0, adding the precipitate solution to a Sepharose 4B column and performing stepwise elution using phosphate buffer and galactose, where an active antiviral material elutes from the column with 0.15 M galactose.

In one preferred embodiment, the cinnamon extract is obtained using the following process:

(i) grinding cinnamon bark into powder and stirring it into an aqueous buffer to obtain a solution;

(ii) centrifuging the solution and separating a supernatant; and

(iii) introducing a salt, such as, for example, a chloride salt, to obtain a precipitate. The process may further comprise of the following steps:

(iv) dissolving the precipitate obtained in step (iii) above in water or buffer at an essentially neutral pH;

(v) separating the solution on a sepharose or Sephadex column; and

(vi) eluting the solution with suitable buffer and varying concentrations of saccharide,

preferably galactose, to obtain the antiviral fractions. In another preferred embodiment, the cinnamon extract is obtained from cinnamon bark, Cinnamomum sp., using the following method:

(i) grinding the bark into powder;

(ii) stirring the bark in aqueous phosphate buffer 0.01 M or 0.02 M, pH 7.0;

(iii) separating the supernatant by centrifugation to be used as the crude neutralizing extract;

(iv) precipitating the active ingredient in the crude extract using 0.15 M KCl or 0.08 M MgCl₂;

(v) dissolving the precipitate in water or 0.01 M phosphate buffer at pH 7.0;

(vi) loading the solution onto a column of sepharose 4B followed by a stepwise elution with phosphate buffer and various concentrations of galactose; and

(vii) eluting the active antiviral material from the column by 0.15 M galactose.

A nutraceutical and/or pharmaceutical composition can be formed using either an effective amount of an extract solution, a separate fraction thereof, a precipitate, a composition comprising the precipitate, and/or combinations thereof, by adding a pharmaceutically or nutraceutically acceptable carrier. Such compositions can also include one, or two or more, of the peptides, nucleic acids, vectors, host cells, or compositions thereof, as disclosed herein. Such compositions can also include other components, such as at least one additional therapeutic or nutraceutic component.

The compounds and/or compositions so formed have antiviral activity. In general, the virus may be an enveloped virus, such as African Swine Fever Virus, Orthomyxoviruses, Paramyxoviruses, Herpesviruses, Retroviruses, Coronaviruses, Hepadnaviruses, Poxviruses, Togaviruses, Flaviviruses, Filoviruses, Rhabdoviruses, and Bunyaviruses. Accordingly, disclosed embodiments also concern a method for treating a viral infection comprising administering to a subject in need thereof a therapeutically effective amount of a cinnamon extract composition, a cinnamon extract precipitate composition, or such compositions when combined with one or more ASFV peptides disclosed herein. Such compositions can be administered by any suitable method as will be understood by a person of ordinary skill in the art, such as orally, nasally, parenterally, subcutaneously and/or intramuscularly.

Certain disclosed embodiments concern a method for producing a neutralized virus for immunization, and a neutralized virus vaccine produced using the neutralized virus. One such embodiment comprises contacting native viruses, such as ASFV, with an effective amount of a cinnamon extract composition and/or a cinnamon extract precipitate composition. Vaccine formulations comprising the neutralized virus can be administered to a subject as discussed above.

Isolated active fractions of cinnamon bark may exhibit absorbance at 280 nm of 15 O.D./mg·cm³. The active fraction remains active even after incubation in acids or bases, such as 0.1 M NaOH, or 0.1 M HCl. Solid active fractions and solutions comprising such active components can be stored at room temperature or below for substantial time period, such as several years. Active precipitate fractions are heat-stable and can be sterilized at temperatures greater than 100° C. and potentially up to at least 134° C.

Compositions of the present invention may protect infected erythrocyte cells from the activity of viruses pre-absorbed on the erythrocytes. Thus, the cinnamon extract of the present invention may be considered as effective treatment of cells already pre-absorbed with the virus. Furthermore, pre-absorption of the cinnamon extract of the invention onto cells may have a prophylactic effect in protecting the cells from subsequent viral infection. Additionally, compositions of the present invention may protect infected erythrocyte cells from the activity of viruses pre-absorbed on the cinnamon extract and/or on one or more other components of one or more compositions disclosed herein, which one or more compositions are then contacted with cells.

The present invention also concerns compositions, which may be nutraceutical or pharmaceutical compositions, comprising the cinnamon extract of the invention together with a pharmaceutically or nutraceutically acceptable carrier. The composition may be in a liquid, solid, or semi solid state.

Furthermore, the present invention concerns a pharmaceutical composition or a nutraceutical composition for the treatment of an infection comprising as an active ingredient an effective amount of the cinnamon extract together with a carrier suitable for pharmaceutical or nutraceutical compositions.

The present invention further concerns a method for treating a subject suffering from viral infection. The method comprises administering to a subject in need of such treatment an effective amount of compositions disclosed herein. The viral infection is preferably an enveloped virus infection; more preferably a virus of the family Orthomyxoviruses, Paramyxoviruses, Herpesviruses, Retroviruses, Coronaviruses, Hepadnaviruses, Poxviruses, Togaviruses, Flaviviruses, Filoviruses, Rhabdoviruses, or Bunyaviruses; most preferably the virus infection is caused by a virus selected from avian influenza virus, Influenza virus, Parainfluenza virus (also referred to herein as “the Sendai virus”), NDV virus (paramyxovirus), HIV viruses, HSV-1 virus, HSFV viruses, ASFV, TILV (orthomyxoviruses) and KHV (herpesvirus).

The active material was isolated by three steps as follows: a) the bark was purchased in the market and was ground into powder before it was stirred in aqueous phosphate buffer 0.01 M-0.02 M, pH 7.0, overnight. The supernatant was separated by centrifugation and was used as the crude neutralizing extract; b) The active material in the crude extract was precipitated by KCl 0.15 M or 0.08M MgCl₂, and the precipitate was dissolved in water or 0.01 M phosphate buffer, pH 7.0 (CE ppt.); c) This solution was submitted onto column of Sepharose 4B followed by a stepwise elution with phosphate buffer and various concentrations of galactose. The active antiviral material may be eluted from the column by 0.15M galactose.

Hemagglutinating unit (HAU) may be determined using 4% washed human red blood cells. Viral hemolytic activity has been tested in vitro by first attaching free virus onto 1 ml of 4% washed human erythrocytes for 15 minutes at room temperature, and then incubating the infected cells in 37° C. for 3 hours followed by centrifugation. The hemolytic activity of the viruses has been determined by measuring the absorbance of the supernatant at 540 nm.

In a particular embodiment, cinnamon extract precipitate may be dissolved in water or in 0.01 M phosphate buffer and added to a 10 ml Sepharose 4B column pre-washed with phosphate buffer 0.01 M at pH 7.0. The column may be washed with the buffer followed by stepwise elution of galactose 0.15 M, 0.3 M, and various concentrations of acetonitrile. An active antiviral material has been found in fraction b eluted from the column by 0.15 M galactose or fraction II.

Various amounts of crude extract have been incubated with 256 HAU samples of Influenza A PR8 virus to test the inhibitory effect on the hemolytic activity of the virus. Hemolytic activity of the virus was totally inhibited by 250 μg of the crude extract.

Various amounts of crude extract have been incubated with 256 HAU samples of Sendai virus to test the inhibitory effect on the hemolytic activity of the virus. Virus alone or the crude extract alone has been used as controls. The hemolytic activity of the virus was totally inhibited by 250 μg of the crude extract.

Cinnamon extract fractions have been dialyzed against water. An active component has been found to have a molecular weight greater than 10 KDa (the dialysis bag cut-off).

In vivo antiviral activity has been determined using mice. Mice have been injected with 250 μl of PBS containing 128 HAU of Influenza A virus alone or Influenza A mixed with 250 μg of the crude extract or the crude extract alone. Mice infected with the virus alone lost weight and most died within 7-10 days. Mice injected with a mixture of the virus and the crude extract continued to gain weight on par with those injected with the crude extract alone. Mice have inhaled 50 μl of water containing 64 HAU of Sendai virus alone, virus mixed with 125 μg of crude extract, or the crude extract alone. The mice were weighed at 2-3 day intervals. Mice infected with the virus alone lost weight and most died within 7-10 days. Mice treated intranasally with a mixture of the virus and the crude extract recovered and gained weight. Each group included 10 mice.

Mice have been injected with 128 HAU of Influenza A PR8 pre-incubated with 250 μg of the cinnamon extract inhibitor for 30 minutes at room temperature. The mice were weighed every 2-3 days for 3 weeks. No deaths occurred among the mice infected with the virus pre-incubated with the inhibitor.

100 PFU aliquots of HSV1 were mixed with 50 μg of a cinnamon extract precipitate according to the present invention. Cells with HSV alone were detached and washed from plate. Cells with HSV mixed with 50 μg cinnamon extract precipitate were not affected. This established that the extracts of the invention protected the Vero cells from HSV-1 infection.

Tests have also established that there is direct correlation between inhibition and increasing amounts of a cinnamon extract and/or cinnamon extract precipitate according to the present invention.

Mice have also been infected with 32 HAU of Sendai virus pre-incubated for 20 minutes with 125 μg of cinnamon extract or a cinnamon extract precipitate, or treated with cinnamon extract or a cinnamon extract precipitate immediately after viral infection. Mice treated with the inhibitor started to gain weight 8 days post infection (P=0.017), whereas the control group which had not been treated with the inhibitor continued losing weight.

Mice have been immunized intranasally with 32 HAU of Sendai virus mixed with 125 μg of a cinnamon extract or a cinnamon extract precipitate. A control group received only water. Three weeks post immunization both groups of mice were infected with 64 HAU of the Sendai virus alone. Immunized mice were not affected by the subsequent virus infection and kept gaining weight (P=0.013).

Mice have been immunized by the Sendai virus mixed with a cinnamon extract or a cinnamon extract precipitate either orally or subcutaneously. Two weeks after a third administration of the virus plus a cinnamon extract or a cinnamon extract precipitate, the mice of both groups were infected with 80 HAU of Sendai virus, as were control mice. Immunized mice were not affected by subsequent virus infection and continued gaining weight and no difference was observed between oral or subcutaneous administration.

HIV-1 activity has been tested on MT2 cells (CD4+T-cells) using the model of syncytia formation in cell culture. 20-120 μl aliquots of cinnamon extract precipitate, 0.5 mg/ml, were incubated with 50 μl virus for 5 minutes in a final volume of 200 μl RPMI medium at room temperature. 90 μl of each mixture were added to the cells in duplicates. After 3 days, syncytia were observed in 95-100% of the control wells without cinnamon extract precipitate and served as the 100% infectivity to which other wells were compared. However, 8-10 μg of cinnamon extract precipitate in 8-10 μl completely neutralized the virus. Inhibition of avian influenza H9N2 by VNF has been tested by an in vitro Hemolysis Assay as done previously (Borkow and Ovadia, 1994, 1999). The hemolytic activity of the influenza virus (release of hemoglobin from red blood cells) was examined on human erythrocytes. Washed diluted erythrocytes were mixed with the virus alone or with a virus preincubated with a cinnamon extract or a cinnamon extract precipitate for 20 minutes at room temperature. Excess virus was removed by washing with PBS, followed by addition of 200 μl of 0.1 M sodium citrate buffer at pH 4.6 for three minutes to fuse the virus with the erythrocytes. The mixture was then washed in PBS, centrifuged and incubated in 0.8 ml PBS at 37 ° C. for 3 hours. Intact erythrocytes were removed by centrifugation and 300 μl aliquots from the supernatant of each sample were placed into wells of an ELISA plate for absorbance measurement in an ELISA plate reader at 540 nm. The hemolytic activity of the virus was neutralized by the cinnamon extract or a cinnamon extract precipitate according to the present invention in a dose dependent manner.

A cinnamon extract or a cinnamon extract precipitate has also inhibited the hemolytic activity of an avian influenza virus after it was attached on the infected cells as it did to the free virus.

Hemagglutinating activity of a Newcastle Disease virus (NDV) has also been tested. Preincubation of the virus (108 EID50) with 10 mg cinnamon extract or a cinnamon extract precipitate according to the present invention resulted in Hemagglutination Inhibition.

In-vivo (In-ova) Neutralization of Avian Influenza H9N2 by a cinnamon extract or a cinnamon extract precipitate has also been tested. One milliliter containing 4.5 mg of a cinnamon extract precipitate according to the present invention and 107 EID50 of influenza H9N2 were incubated for 20 minutes at room temperature before preparing 10-fold dilutions from this mixture. 0.1 ml of each dilution was injected into each allantoic cavity of 10 embryonated chicken SPF eggs. Dilutions of the virus alone or cinnamon extract precipitate were used as controls (10 eggs in each group). The cinnamon extract precipitate decreased the viral infectivity by 5 logs and increased the embryo survival at a similar rate.

In vivo neutralization of Newcastle Disease Virus by a cinnamon extract or a cinnamon extract precipitate has also been tested. One ml containing 5 mg of a cinnamon extract precipitate according to the present invention and 108 EID⁵⁰ of Newcastle Disease Virus was incubated for 20 minutes at room temperature before preparing 10-fold dilutions from this mixture. 0.1 ml of each dilution was injected into each allantoic cavity of 10 chicken SPF eggs. Virus alone and a cinnamon extract or a cinnamon extract precipitate alone were used as controls. A cinnamon extract or a cinnamon extract precipitate decreased the viral infectivity by 5 logs and increased embryo survival similarly.

The serum titer of chicks following vaccination with NDV in combination with a cinnamon extract precipitate has been reviewed. In ovo vaccination of a first group was performed by injecting 0.1 ml of PBS containing 105.3 EID⁵° of NDV preincubated with 1 mg of VNF into SPF chicken eggs at day 18 of the embryonic development. A second group was intraocularly vaccinated 1-2 days after. Non-vaccinated chicks were used as controls. Blood samples were withdrawn periodically and serum titer was determined by hemagglutination inhibition assay of serial dilutions of each serum. Serum titer after in ovo vaccination was as good as intraocular vaccination.

### VIII. Examples

The following examples are provided to illustrate certain features and/or embodiments of the disclosure. These examples should not be construed to limit the disclosure to the particular features or embodiments described. Changes therein and other uses that are encompassed within the spirit of the invention as defined by the scope of the claims will occur to those of ordinary skill in the art.

### Example 1

### Peptide Prediction and Synthesis

This example describes a method for predicting putative peptides immunogenic against ASFV and for synthesizing the peptides for efficacy studies, both in vitro and in vivo.

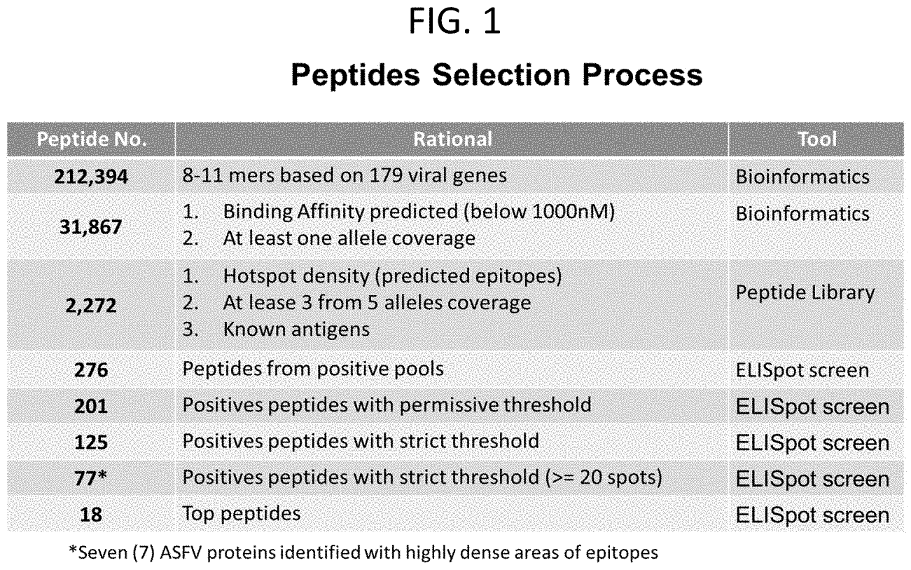

The complete genome of the ASFV China/2018/AnhuiXCGQ strain (GenBank Accession No. MK128995.1) was screened for CD8+epitopes in relation to the known SLA class I alleles of the Yorkshire, Landrace, and Duroc swine breed lines. Candidate peptides were evaluated according to four criteria: (1) predicted binding affinity of the peptide to SLA class I molecules; (2) position in highly dense clusters of putative epitopes as a method to enrich positive responders; (3) coverage of SLA alleles and prioritization of highly prevalent alleles; and (4) the nature of the source protein (giving precedence to immunogens). Out of 212,394 putative peptides, 2,272 were selected for further evaluation (FIG. 1).

First, a total of 49 SLA alleles found in the Yorkshire, Landrace, and Duroc breed lines were identified and functionally clustered into 29 supertypes using the WIC cluster tool. In the case of functional overlap, one representative allele from the given supertype was chosen for use in peptide binding predictions (representative alleles are shown in bold in Table 3). The choice of the representative allele was based on the prediction accuracy value generated by the cluster mapping analysis. A computational analysis was conducted to identify peptides predicted to bind to the SLA class I molecules using the entire ASFV China/2018/AnhuiXCGQ strain proteome (179 open reading frame products). The NetMHCpan-4.0 algorithm predicts peptide interactions with WIC class I molecules by integrating in silico-derived binding affinity information and eluted ligands derived from MS data (Jurtz, et al. J. Immunol 199(9):3360-3368, 2017). Thus, the NetMHCpan-4.0 algorithm may generate peptides predicted to be immunogenic against ASFV. This algorithm was used to predict the binding affinities of 8, 9, 10, or 11 amino acid-long peptides derived from the 179 open reading frames of the ASFV China/2018/AnhuiXCGQ strain (GenBank Accession No. MK128995.1) (a total of 212,394 peptides) for each of the 29 representative alleles shown in bold in Table 3. Out of 212,394 peptides, 31,868 peptides had an allelic coverage of one or more supertypes (FIG. 1).

TABLE 3
Relevant SLA alleles functionally clustered into 29 supertypes,
with representative alleles shown in bold.

| | | Prediction |
|---|---|---|
| Supertype | Allele | accuracy |
| S1 | SLA-3: 0101 | 0.879 |
| S1 | SLA-1: 1103 | 0.66 |
| S2 | SLA-3: 0601 | 0.868 |
| S2 | SLA-3: 0701 | 0.954 |
| S2 | SLA-3-0401 | 1 |
| S2 | SLA-3: 0402 | 0.912 |
| S2 | SLA-3-0404 | 0.868 |
| S3 | SLA-2: 060201 | ND |
| S4 | SLA-3: 0306 | 0.587 |
| S4 | SLA-3: 0502 | 0.739 |
| S4 | SLA-3: 0503 | 0.739 |
| S4 | SLA-3: 0506 | 0.739 |
| S5 | SLA-2: 1603 | ND |
| S6 | SLA-1: 0703 | 0.749 |
| S6 | SLA-1: 0705 | 0.811 |
| S7 | SLA-1: 0811 | 0.582 |
| S8 | SLA-2: 1005 | ND |
| S9 | SLA-1: 0806 | 0.663 |
| S9 | SLA-1: 0807 | 0.683 |
| S10 | SLA-2: 1002 | 0.656 |
| S11 | SLA-1: 1401 | 0.572 |
| S12 | SLA-2: 0903 | ND |
| S13 | SLA-2: 1001 | 0.589 |
| S13 | SLA-2: 1004 | ND |
| S14 | SLA-1: 0901 | 0.711 |
| S15 | SLA-2: 0202 | 0.647 |
| S16 | SLA-1: 0704 | 0.615 |
| S17 | SLA-1: 0805 | 0.584 |
| S17 | SLA-2: 1006 | ND |
| S17 | SLA-1: 0808 | 0.67 |
| S18 | SLA-1: 0701 | 0.653 |
| S18 | SLA-1: 0702 | 0.653 |
| S19 | SLA-1: 0401 | 1 |
| S19 | SLA-1: 1301 | 0.842 |
| S19 | SLA-1: 1701 | 0.824 |
| S20 | SLA-1: 0801 | 0.791 |
| S20 | SLA-1: 0812 | 0.791 |
| S21 | SLA-2: 0401 | 1 |
| S21 | SLA-2: 0402 | 0.903 |
| S22 | SLA-1: 1501 | 0.741 |
| S23 | SLA-1: 1201 | 0.727 |
| S23 | SLA-2: 0102 | 0.776 |
| S24 | SLA-2: 0101 | 0.683 |
| S25 | SLA-2: 0501 | 0.667 |
| S25 | SLA-2: 0503 | 0.574 |
| S26 | SLA-2: 0504 | ND |
| S27 | SLA-1: 0101 | 0.771 |
| S28 | SLA-2: 0502 | 0.688 |
| S29 | SLA-2: 0505 | ND |

The SLA-1*0401, SLA-2*0402, SLA-3*0402, SLA-1*0702, and SLA-2*0502 alleles are highly prevalent in the swine population, including within the Duroc, Yorkshire, and Landrace breed lines. The computationally-determined 31,867 peptides were tested for coverage of these five common alleles, and a total of 2,559 peptides provided coverage of at least three of the five alleles. To further reduce the number of peptides for evaluation, only peptides covering at least 15 alleles in general (of the 49 SLA alleles relevant to the Yorkshire, Landrace, and Duroc breed lines) were selected from the list of the 2,556. This 1,190 peptide list was denoted as Subset C (peptides selected by Coverage).

The 31,868 peptides predicted to bind SLA class I molecules were further used in a cluster mapping analysis conducted using the HotSpots program package developed at the Israel Institute for Biological Research. A cluster was defined as a peptide having a minimum length of 8 amino acids (the shortest predictive peptide length) and a maximum length of 25 amino acids. Clusters contained two or more peptides, with each peptide overlapping or in tandem with another peptide. The mapping analysis generated 9,654 clusters containing 31,815 unique peptides (after removal of duplications due to overlap between cluster regions). Cluster density was defined and calculated as the number of epitopes per unit length, and densities obtained were in the range of 0.11-1.56. Peptides located in high density (1.21-1.56) clusters were selected for further analysis. The 524 selected peptides were designated as Subset H (peptides selected from HotSpots).

Certain ASFV proteins are known immunogens and/or are involved in immune modulation and/or virulence in swine. Therefore, peptides 8-11 amino acids long derived from 17 such ASFV proteins (a total of 2,666 peptides) were assessed for allelic coverage. Peptides covering at least one of the five prevalent alleles and at least six of the 49 SLA alleles relevant to the Yorkshire, Landrace, and Duroc breed lines were selected for further characterization. These 750 peptides were denoted as Subset A (peptides selected from Antigens).

The final list of putative epitopes for experimental evaluation was compiled from the three subsets described above (subsets C, H, and A). After removal of redundancies (peptides common to two or more subsets, or redundant within a subset), the final list consisted of 2,272 unique peptides (FIG. 1).

Peptides can be synthesized using one or more synthetic chemical methods and/or can be obtained from intracellular synthesis using one or more recombinant techniques. In this example, peptides predicted to be immunogenic against ASFV are synthesized using a solid-phase method wherein the C-terminus of the first amino acid is coupled to an activated solid support, such as polyacrylamide. The carboxyl group of an incoming amino acid is coupled to the N-terminus of the growing amino acid chain (C-N synthesis). Step-wise synthesis adds amino acids one at a time to each peptide chain. Chemical groups are employed to block non-specific reactions during peptide synthesis. C-terminal carboxylic acids on incoming amino acids are activated using carbodiimides, and 1-hydroxybenzotriazole (HOBt) is used to reduce the risk of racemization during amino acid coupling. At the completion of the synthesis of a given peptide, protective groups are removed using acidolysis. Synthesized peptides are purified using reverse-phase chromatography, and >90% purity is established.

### Example 2

### Peptide Validation

This example describes an efficient in vitro method for screening putative peptides immunogenic against ASFV. The peptides described in this example are predicted using bioinformatics methods and then produced using chemical synthesis methods as described in Example 1. Using the in vitro selection process described in this example, the number of potential epitopes is reduced to allow further evaluation of a workable number of only the most promising candidates.

Synthesized peptides predicted to be immunogenic against ASFV are screened against peripheral blood lymphocytes in ELISpot assays, which allow detection (at a single-cell level) of interferon secretions from previously-exposed lymphocytes (lymphocytes collected from swine exposed to ASFV) in reaction to the peptides. Peripheral blood lymphocytes were collected from swine that had been previously challenged with low doses of attenuated ASFV China/2018/AnhuiXCGQ strain or had been exposed to live ASFV China/2018/AnhuiXCGQ. ASFV used to challenge the swine was propagated in primary porcine alveolar macrophages and quantified using qPCR and hemadsorption assays.

ELISpot assays to detect interferon-gamma (IFN-γ) were performed in microplates. Ready-to-use porcine IFN-γ ELISpot assay kits are commercially available from multiple suppliers. An antibody specific for porcine IFN-γ was pre-coated onto a PVDF-backed microplate. Lymphocytes (previously exposed to ASFV) stimulated with a given synthetic peptide were pipetted into the wells of the microplate, and IFN-γ secreted by the stimulated cells was captured by the immobilized antibodies in the immediate vicinity of each cell. Cells were removed from the wells by washing and the IFN-γ-bound, immobilized antibodies were incubated with a biotinylated detection antibody, followed by alkaline-phosphatase conjugated to streptavidin. A dark blue-to-black colored precipitate formed at each location in the wells where the immobilized antibodies had bound IFN-γ secreted by the stimulated cells. The resultant spots were counted using an automated plate reader designed for this purpose.

Analysis of ELISpot assay results for each of the peptides predicted to be immunogenic against ASFV identified a significantly smaller number of candidate peptides that each produce a strong immune response above specific thresholds set by this study. These candidates are considered the most promising peptides for further development of compositions to stimulate an immune response against ASFV in swine or to immunize swine against ASFV.

Two analyses were conducted in this study: a “full screen” that assessed all 2,272 of the bioinformatically-identified candidate peptides, and a “pool screen” conducted using pools of peptides containing eight or nine peptides per pool. The full screen was conducted using lymphocytes collected from two swine, denoted 9H (animal 9 from farm H) and 14S (animal 14 from farm S). The negative control (NC) background was used to calculate a permissive threshold and a strict threshold as follows:

Permissive threshold (PT) =average of medium+2* STDEV_P

Strict threshold (ST) =average of medium+5*STDEV_P

wherein “average of medium” denotes the average number of spots in wells with medium only, calculated for each swine plate separately, and “STDEV_P” denotes standard deviation based on the entire population. The threshold values calculated for each swine are shown in Table 4. “Positive” peptides (i.e., peptides with spot numbers above a threshold value) were considered the most promising peptides for further development and experimental analysis.

TABLE 4
Permissive and stringent threshold values (spot number) for each swine.

| | Permissive | Stringent |
|---|---|---|
| Swine | Threshold | Threshold |
| 10S | 10 | 18 |
| 14S | 12 | 20 |
| 2S | 17 | 28 |
| 3H | 15 | 23 |
| 5H | 13 | 18 |
| 6H | 12 | 20 |
| 7H | 14 | 20 |
| 7S | 2 | 3 |
| 8H | 13 | 19 |

Table 5 shows the number of positive peptides identified in the full screen of all 2,272 bioinformatically-identified peptides using lymphocytes collected from animals 14S and 9H. Positive peptides identified in the full screen, along with ELISpot assay results (number of spots counted for each peptide), are shown in Appendices II (animal 14S) and III (animal 9H). Thirteen positive peptides were identified above the permissive threshold as shared in both animal 14S and animal 9H, while 46 were unique to swine 9H and 198 were unique to swine 14S. No positive peptides above the stringent threshold were identified as shared; 14 were unique to swine 14S and seven were unique to swine 9H.

TABLE 5
Number of Positive Peptides Identified in the Full Screen

| | Number of Positive Peptides | |
|---|---|---|
| | Swine 14S | Swine 9H |
| | (PT = 12, | (PT = 16, |
| Threshold | ST = 20) | ST = 26) |
| Permissive (PT) | 211 | 59 |
| Stringent (ST) | 14 | 7 |

The first pool screen was conducted with pools of 8-9 peptides selected from the 2,272 peptides and used lymphocytes from 9 swine denoted 3H, 5H, 6H, 7H, 8H, 2S, 7S, 10S, 14S. This first pool screen identified 238 total “positive” peptide pools (i.e., peptide pools with spot numbers above the threshold value) above the permissive threshold and 128 above the stringent threshold. Table 6 shows the number of positive peptide pools (each containing eight or nine peptides) identified in each swine (threshold values are shown for each swine in Table 4).

TABLE 6
Number of Positive Peptide Pools Identified in
Each Swine Above Each Threshold.

| | Permissive | Stringent |
|---|---|---|
| Swine | Threshold | Threshold |
| 10S | 26 | 11 |
| 14S | 117 | 9 |
| 2S | 37 | 6 |
| 3H | 3 | 1 |
| 5H | 77 | 20 |
| 6H | 26 | 10 |
| 7H | 84 | 12 |
| 7S | 125 | 92 |
| 8H | 70 | 14 |

Table 7 shows the number of pools that were identified as positive in one or more animals.

TABLE 7
Number of Pools Identified as Positive in One or
More Animals Above Each Threshold.

| Number | Permissive | Stringent |
|---|---|---|
| of Swine | Threshold | Threshold |
| 8 | 1 | — |
| 7 | 2 | 1 |
| 6 | — | — |
| 5 | 12 | 1 |
| 4 | 20 | 1 |
| 3 | 63 | 6 |
| 2 | 74 | 22 |
| 1 | 66 | 97 |

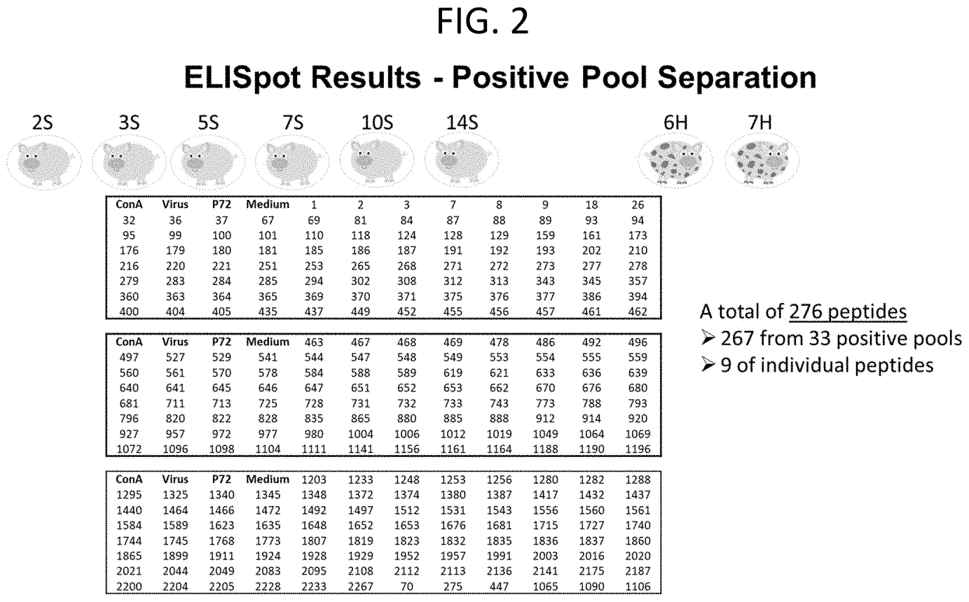

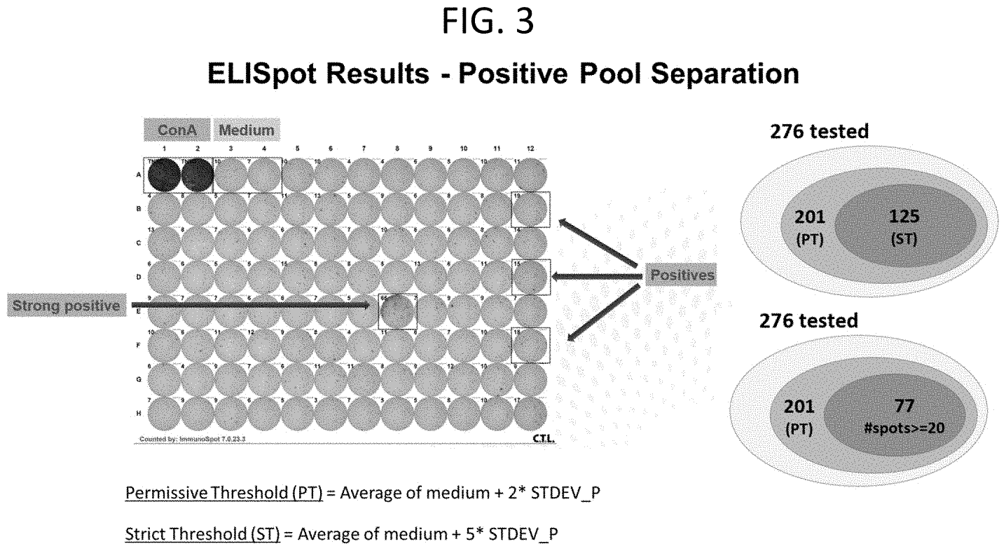

Thirty-three of the 238 positive pools above the permissive threshold were selected for further analysis. Of these 33 pools, 22 were selected because they exhibited cross reactivity in at least five of eight selected pigs (3S, 5S, 14S, 6H, 7H, 2S, 7S and 10S), eight pools were selected because they exhibited cross reactivity in seven of the 15 total pigs screened, and three pools were selected because they each contained at least three individual peptides shown to react with pig 14S in the full screen. A total of 276 peptides from 33 positive pools were assessed individually via ELISpot screening, using concanavilin A (ConA) as a positive control (FIGS. 2 & 3). Of these 276 peptides, 201 were identified above the permissive threshold (Appendix IV), and of the 201 peptides, 125 were identified above the stringent threshold (FIG. 3, Appendix VIII). Further, 77 of the peptides identified above the stringent threshold produced greater than or equal to 20 spots in the ELISpot assays (FIG. 1, Appendix V). Of these 77 peptides, the 18 peptides that produced the most spots in the ELISpot assays were designated “top” peptides (FIG. 1, Appendix VI).

### Example 3

### Immunogenic Construct Assembly and Expression

This example describes the assembly and expression of amino acid constructs comprising one or more peptides of SEQ ID NOs. 2-2273, one or more “domains” as defined in this Example below, and/or one or more full- or partial-length amino acid sequences encoding one or more ASFV immunogenic proteins.

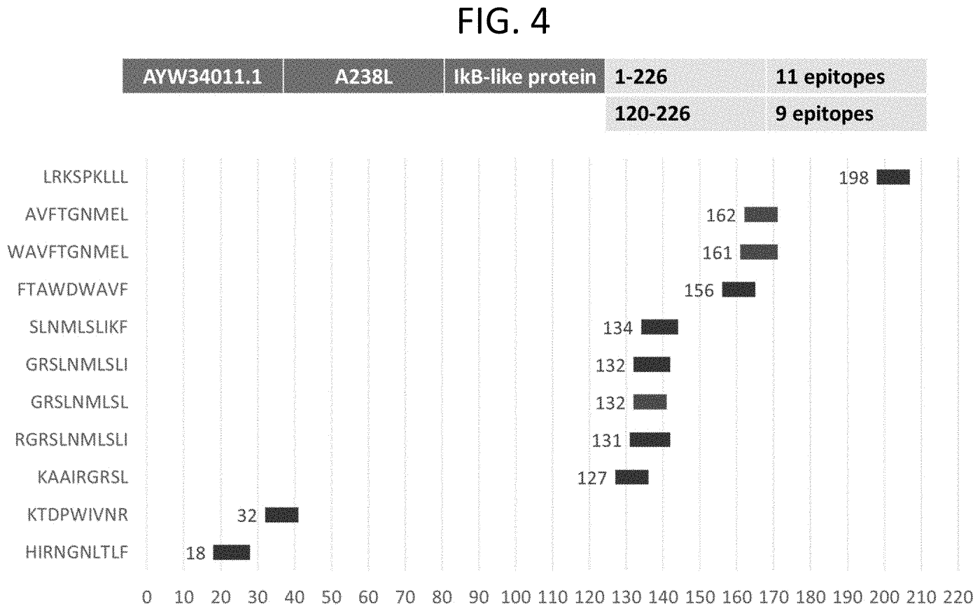

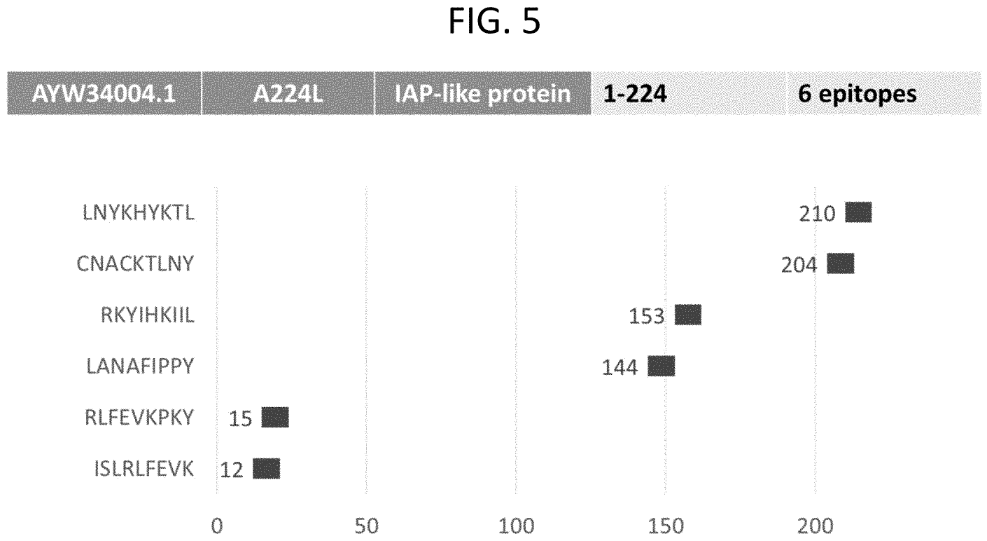

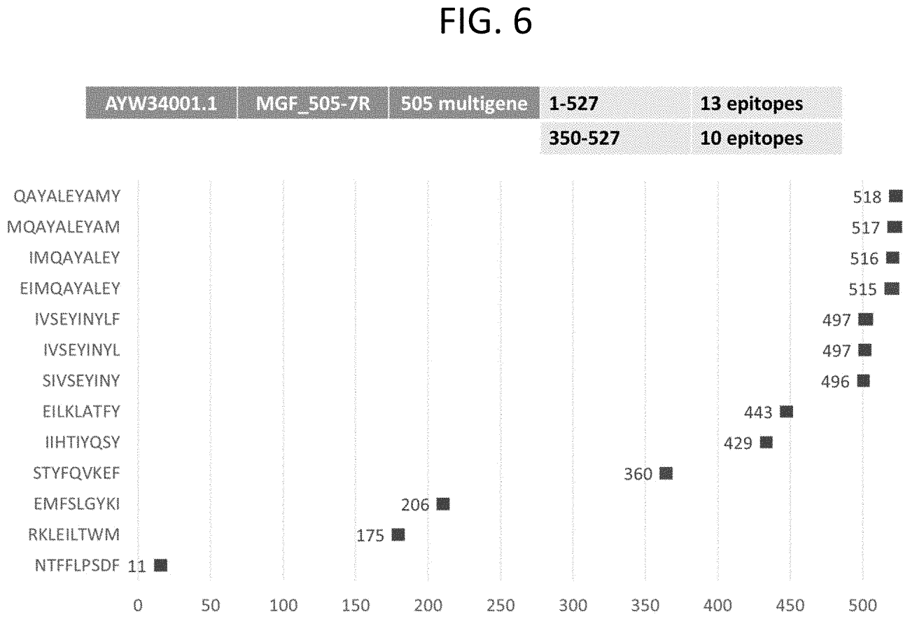

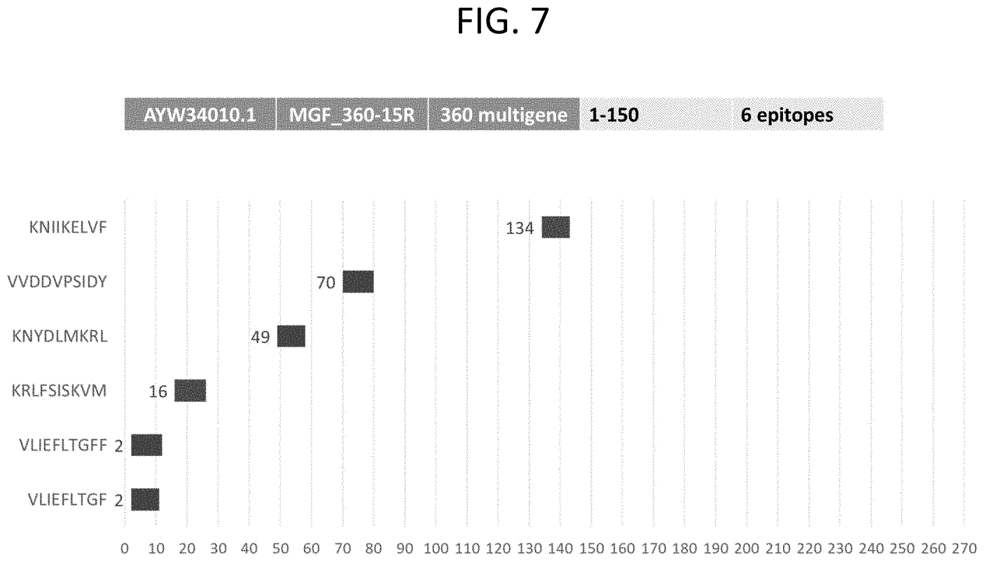

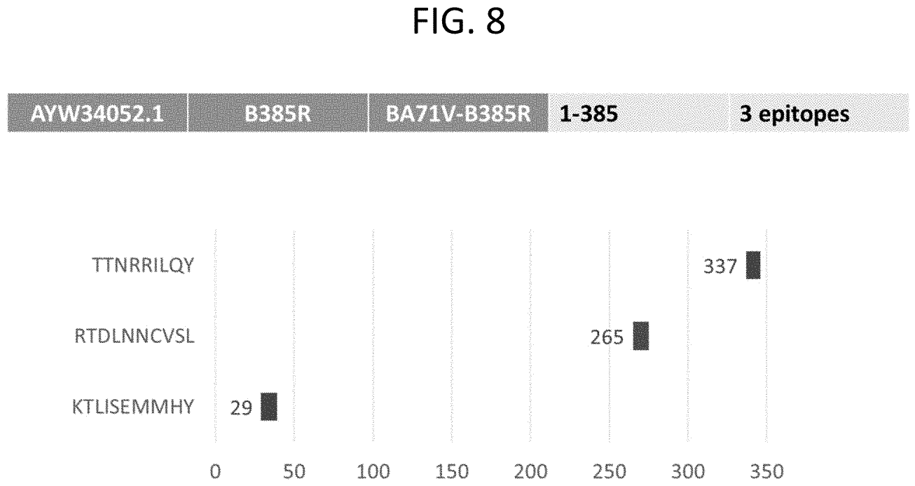

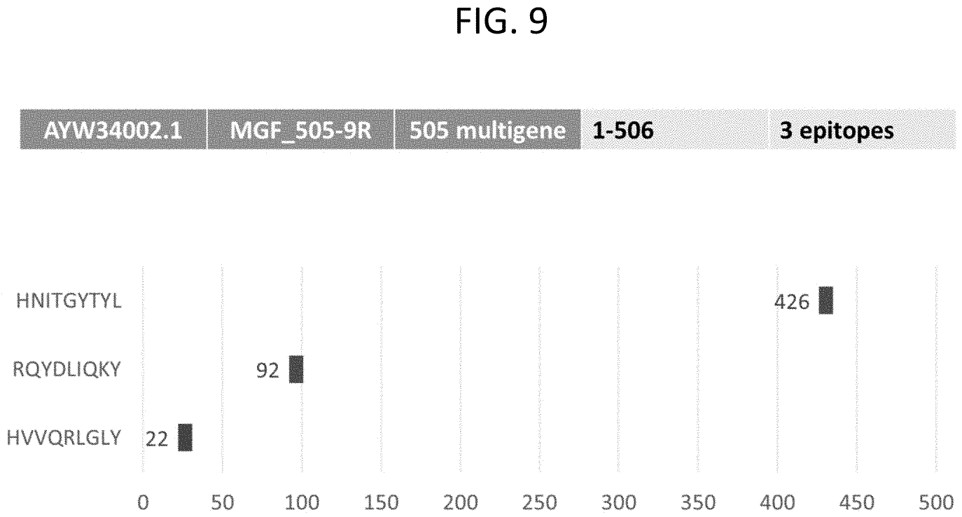

As described in Example 2, 77 peptides identified above the stringent threshold in ELISpot assays produced greater than or equal to 20 spots per well (FIG. 1, Appendix V). These 77 peptides were mapped to their locations within ASFV proteins (Appendices V-VI). Forty-four of the 77 peptides were clustered (Appendix VII) within the following seven ASFV proteins having GenBank Accession Nos.: AYW34011.1 (A238L, containing I⁻KB-like ankyrin repeats; FIG. 4; SEQ ID NOs. 2366-2367), AYW34004.1 (A224L, IAP-like protein p27; FIG. 5; SEQ ID NOs: 2368-2369), AYW34001.1 (MGF_505-7R; FIG. 6; SEQ ID NOs. 2370-2371), AYW34010.1 (MGF_360-15R; FIG. 7; SEQ ID NOs. 2372-2373), AYW34052.1 (zinc finger protein B385R; FIG. 8; SEQ ID NOs. 2374-2375), AYW34002.1 (MGF_505-9R; FIG. 9; SEQ ID NOs. 2376-2377), and AYW33963.1 (MGF_110-3L; FIG. 10; SEQ ID NOs. 2378-2379). “Domains” in this Example are regions of peptide clustering (also known as “hotspots”) within the seven ASFV proteins.

Various combinations of one or more peptides selected from Appendix VII, one or more ASFV domains (“hotspots” as shown in SEQ ID NOs. 2331-2335), and/or one or more full- and/or partial-length ASFV immunogenic proteins (as shown in SEQ ID Nos. 2323-2329 and/or nucleic acids of SEQ ID NOs. 2339-2345) were assembled into the expression constructs of SEQ ID NOs. 2310-2330. Each construct included a histidine tag (His-tag) at the C-terminus to detect expression via western blot analysis, and an N-terminus linker sequence (GSSG) and HiBiT sequence (GSGWRLFKKLS) for expression detection in lysate. Certain constructs comprising peptides also include spacer sequences (GPGPG or AAY) between individual peptide sequences. Constructs comprising peptide sequences selected from the 44 peptides found to cluster within the seven ASFV proteins (Appendix VII), also included HLT, Sumo, or maltose binding protein (MBP) sequences at the N-terminus in order to support expression. Exemplary Sumo and MBP sequences are provided in SEQ ID NOs. 2336 (corresponding to and 2337 (corresponding to NP_418458.1, which is incorporated by reference herein), respectively. For constructs with a MBP fusion protein, MBP was synthetically cloned into the twist vector using the MBP sequence from the pMAL-c2 vector. For constructs with a Sumo fusion protein, Sumo was synthetically cloned into the twist vector using the Sumo sequence from the Champion pET SUMO vector (Thermofisher). The HLT protein is the lipoyl domain from Bacillus Stearothermophilus E2p (SEQ ID NO. 2338), along with an N-terminus His-tag and an optimized Tobacco Etch Virus (TEV) protease cleavage site. Additional information about the lipoyl domain from B. Stearothermophilus E2p (SEQ ID NO. 2338) can be found in Packman et al., Amino acid sequence analysis of the lipoyl and peripheral subunit-binding domains in the lipoate acetyltransferase component of the pyruvate dehydrogenase complex from Bacillus stearothermophilus, Biochem. 1, 1988, 252:79-86, which is incorporated by reference in its entirety herein. Additional information concerning the HLT fusion protein and similar fusion proteins that can enhance solubility of proteins that include natively disordered regions can be found in Lebediker & Danieli, Production of prone-to-aggregate proteins, FEBS Letters, 2014, 588(2):236-246, which is incorporated by reference in its entirety herein. Constructs 55 and 56 were purchased in a twist cloning vector for use in a pseudorabies virus vector.

Each construct was expressed in E. coli at 22° C. and 37° C. For each construct independently, polyethylene glycol (PEG) competent E. coli cells were transformed using a heat shock method. Briefly, 100 μL of competent cells was transferred to tubes on ice. A plasmid comprising a given construct was added to a tube and the mixture was incubated at 4° C. on ice, followed by 45 seconds at 42° C., and 2 minutes on ice. Room temperature SOC medium (0.9 mL) was added to the tube, which was then incubated in a shaker at 37° C. for between one hour and 90 minutes. Transformed cells were then plated (100 μL per plate. Plates can contain appropriate selection antibiotics depending on the vector used.

While cell growth at 22° C. promotes soluble expression such that expression products can be collected from culture supernatants, growth at 37° C. promotes expression of the constructs in inclusion bodies, which can be collected as a component of the cell pellet. Levels of expression for each construct were assessed by isolating proteins from culture supernatants and from pelleted cells. Briefly, proteins in inclusion bodies were isolated from cells using the following protocol: Cell pellets were washed first with Triton X-100, second with Triton X-114, third with 1% CHAPS reagent, and fourth with 6 molar urea. Pellets were then frozen and stored at -80° C. prior to use.

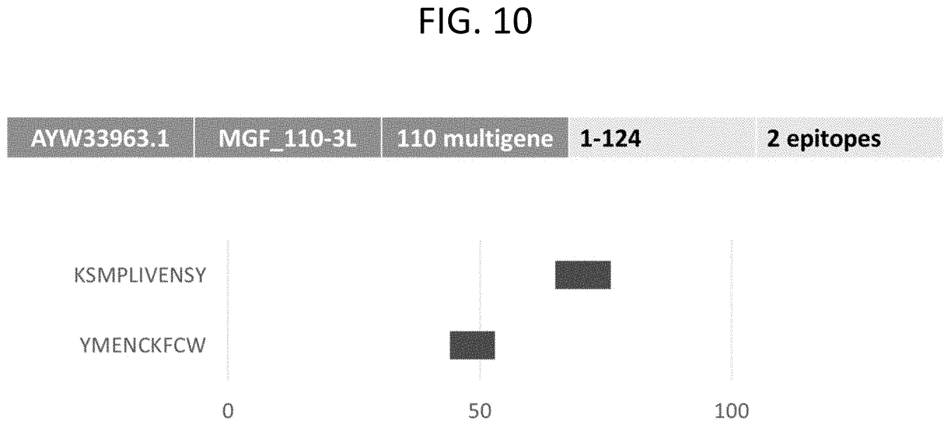

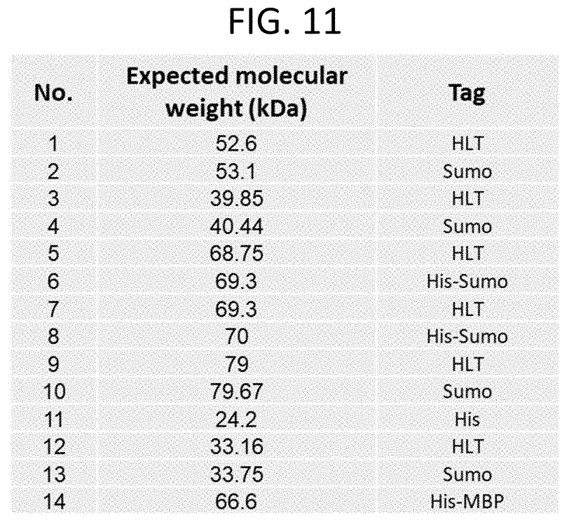

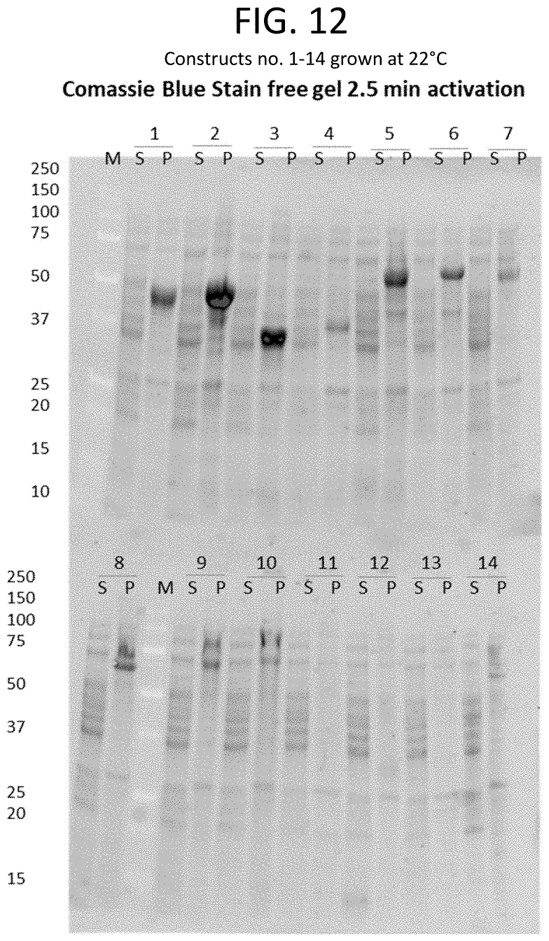

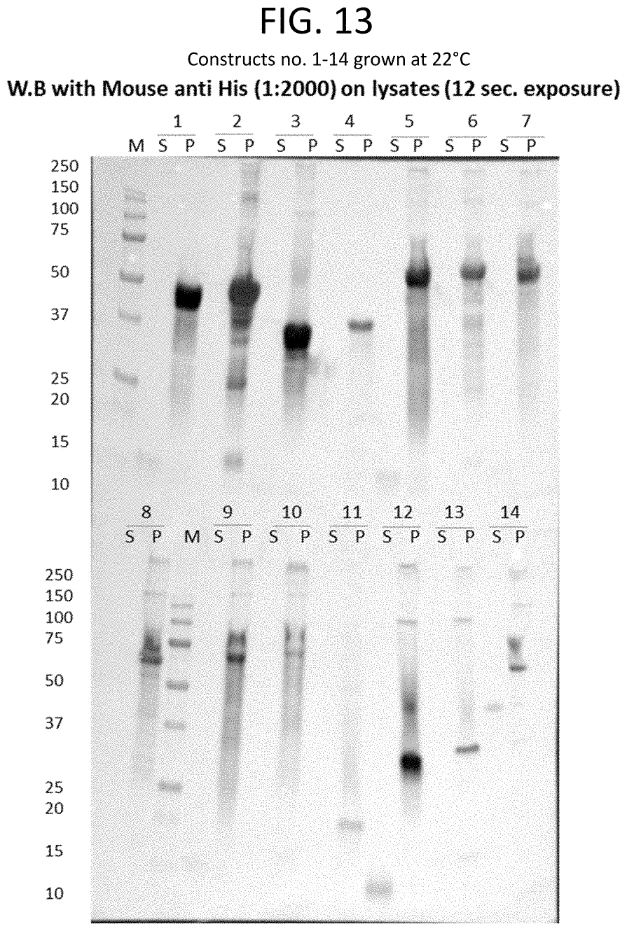

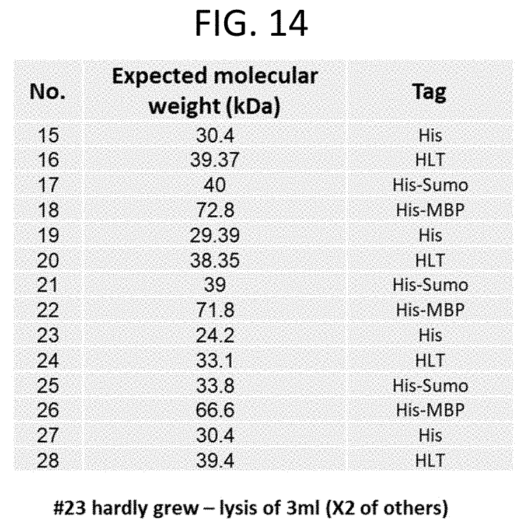

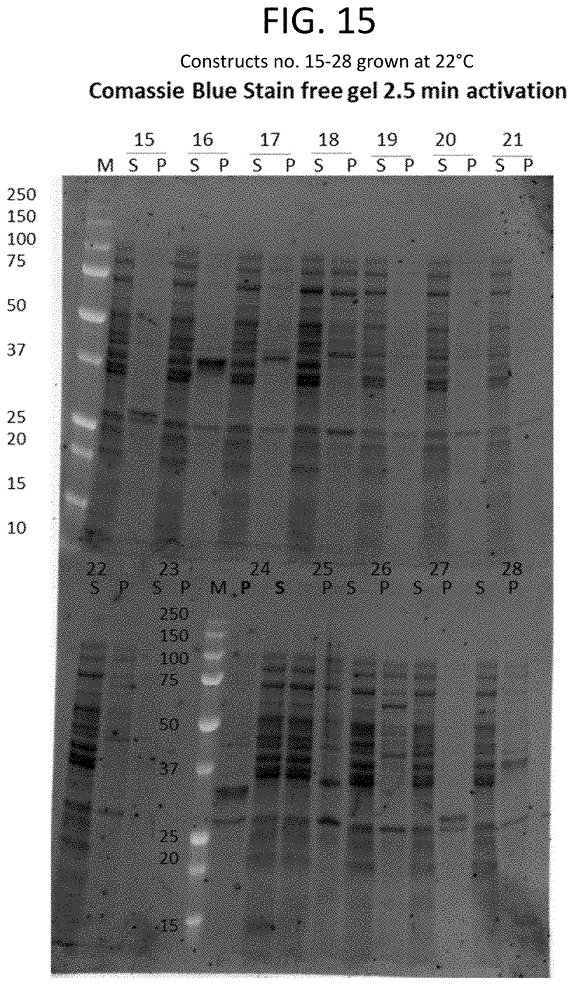

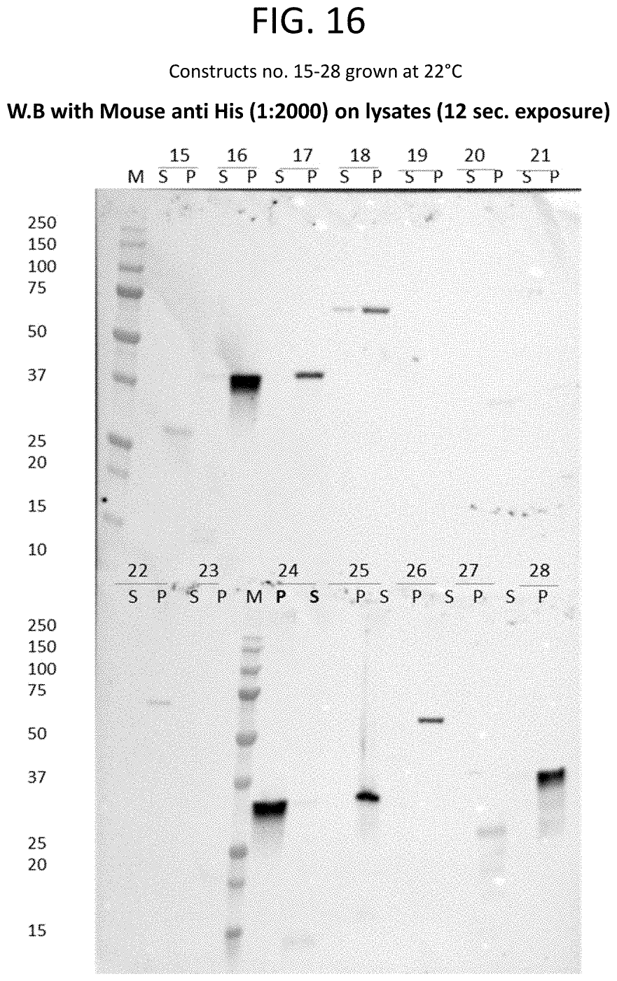

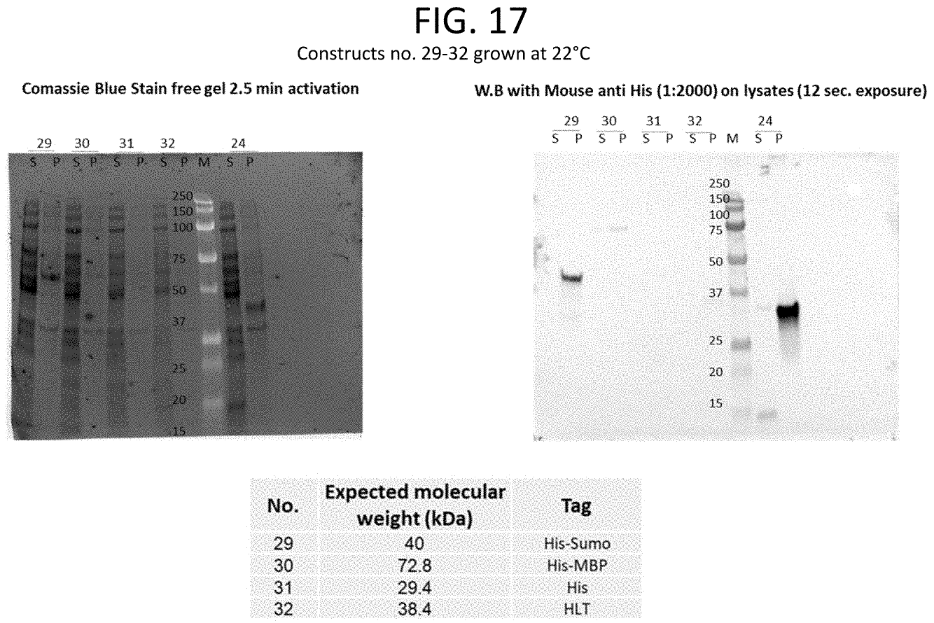

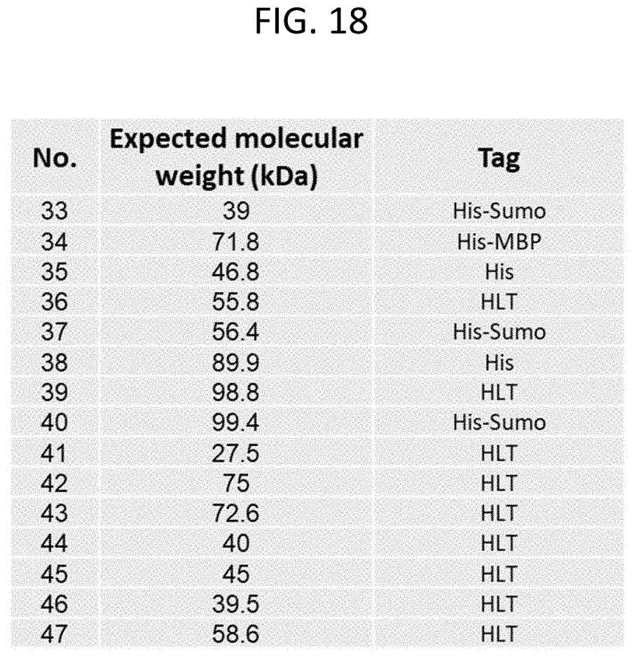

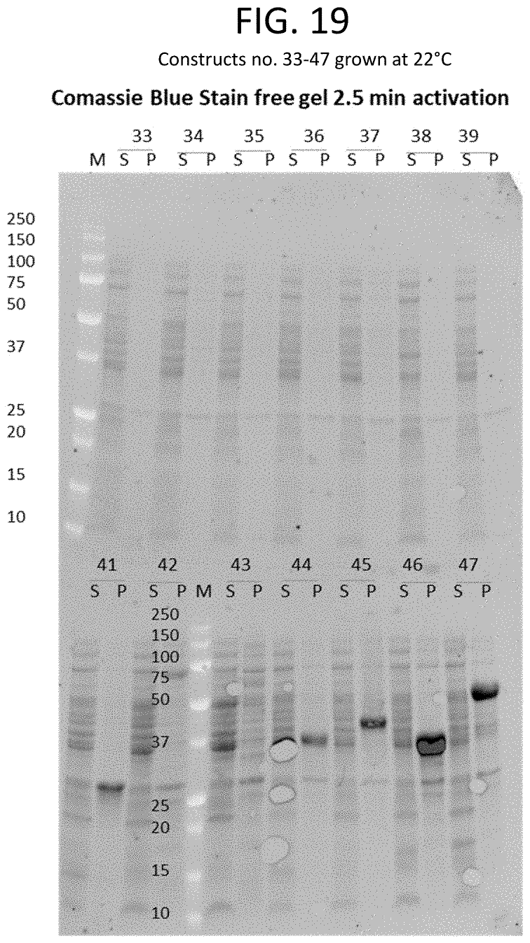

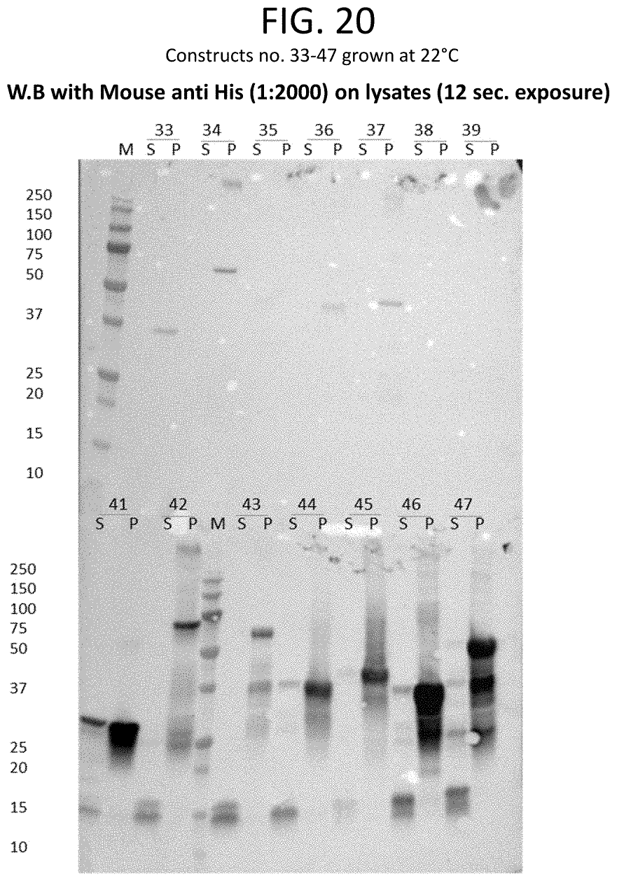

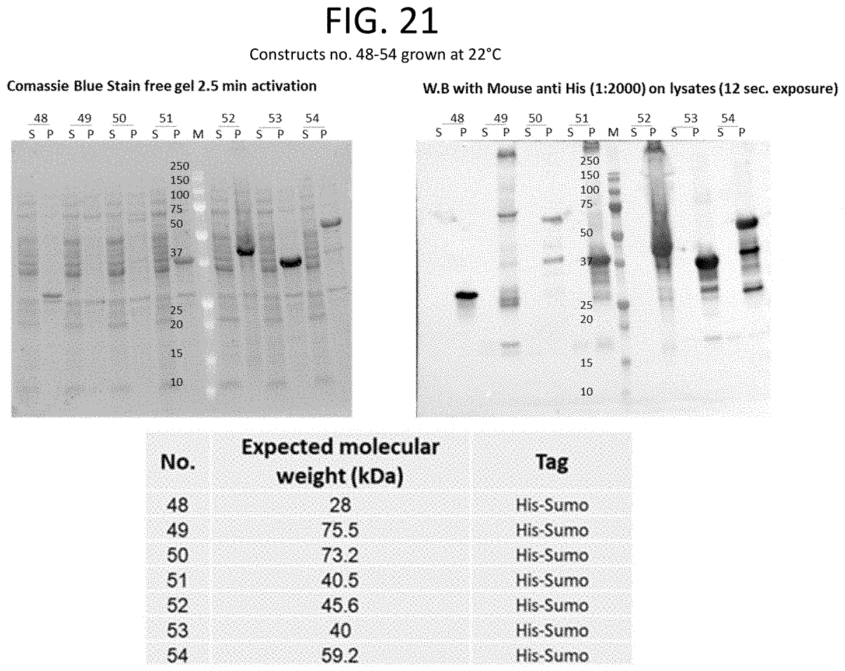

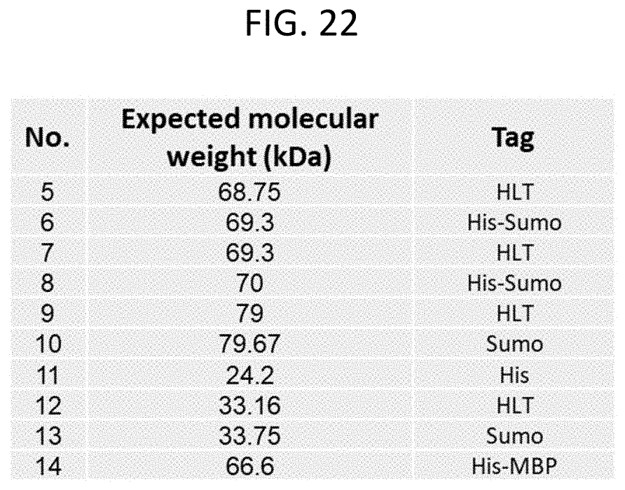

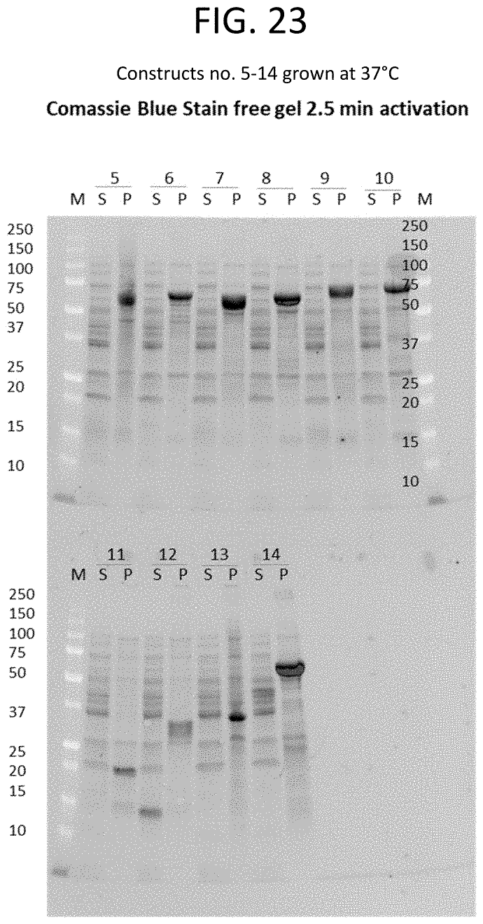

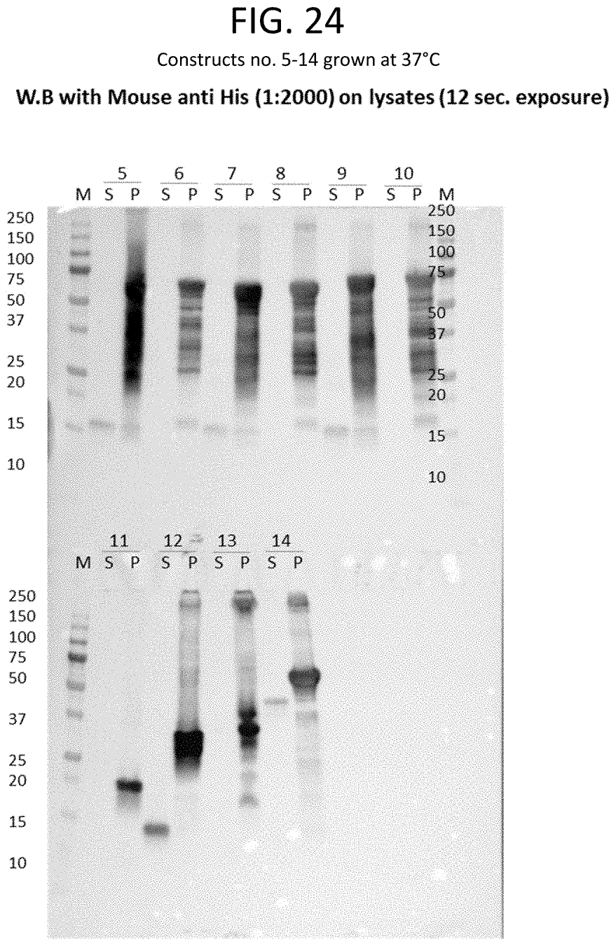

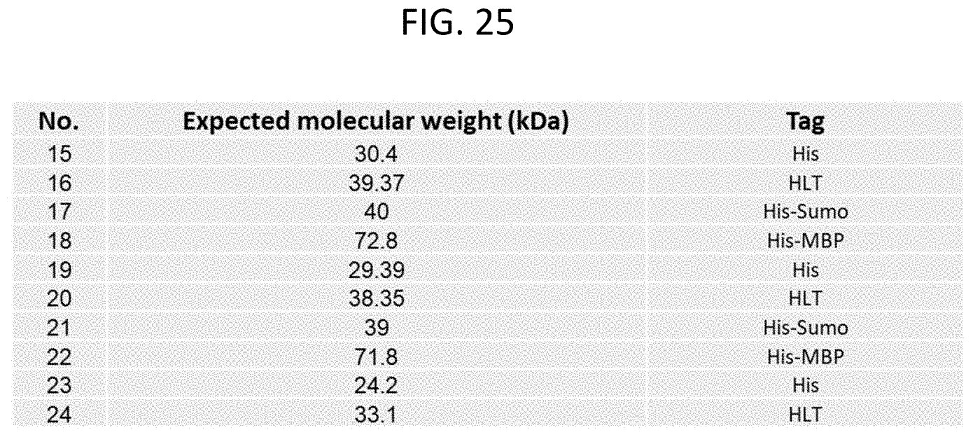

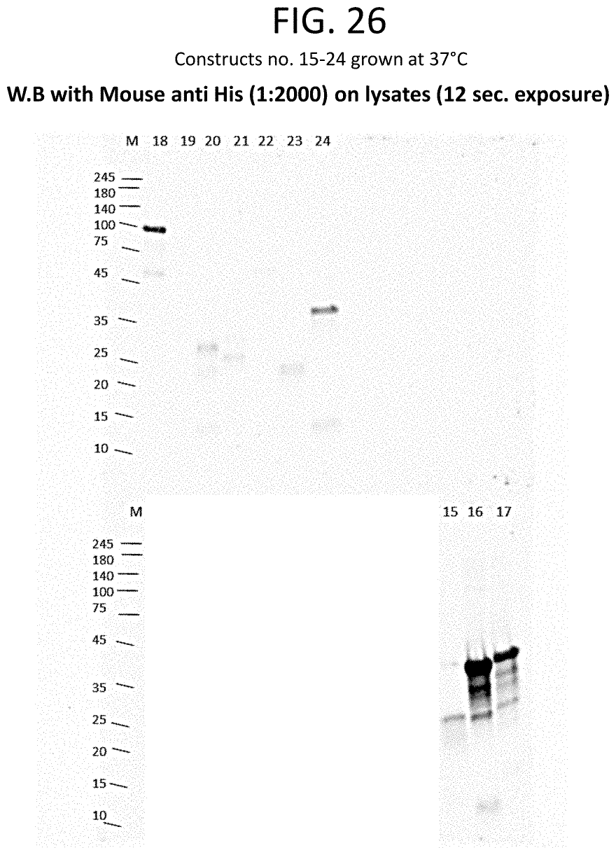

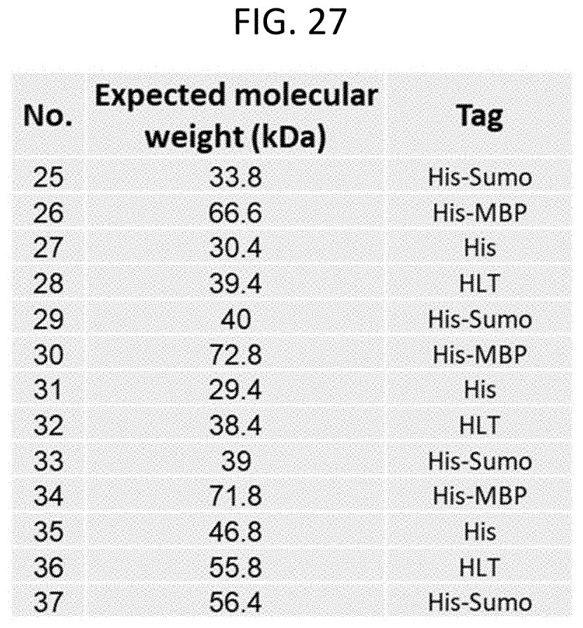

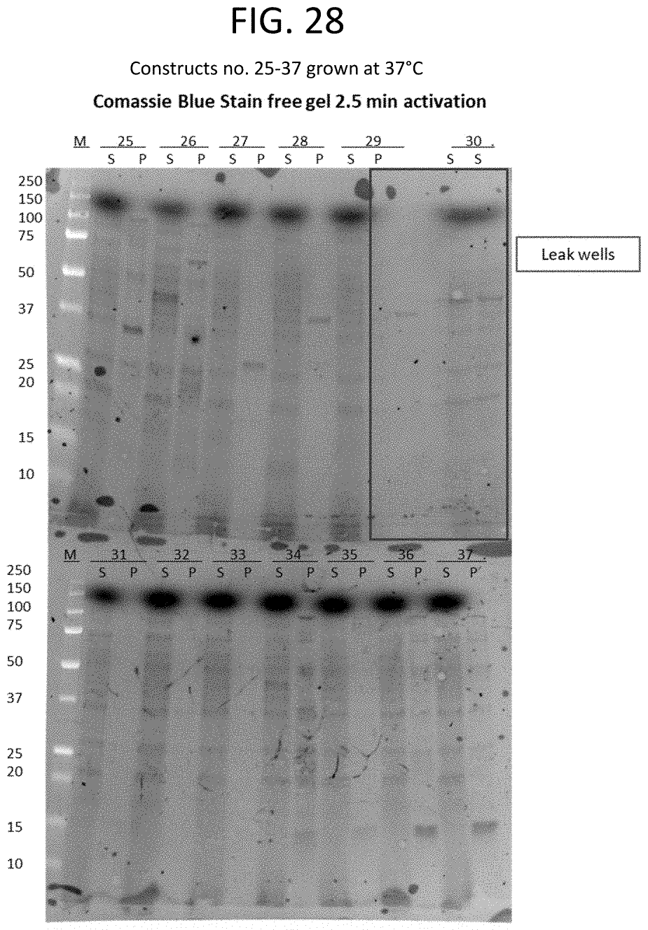

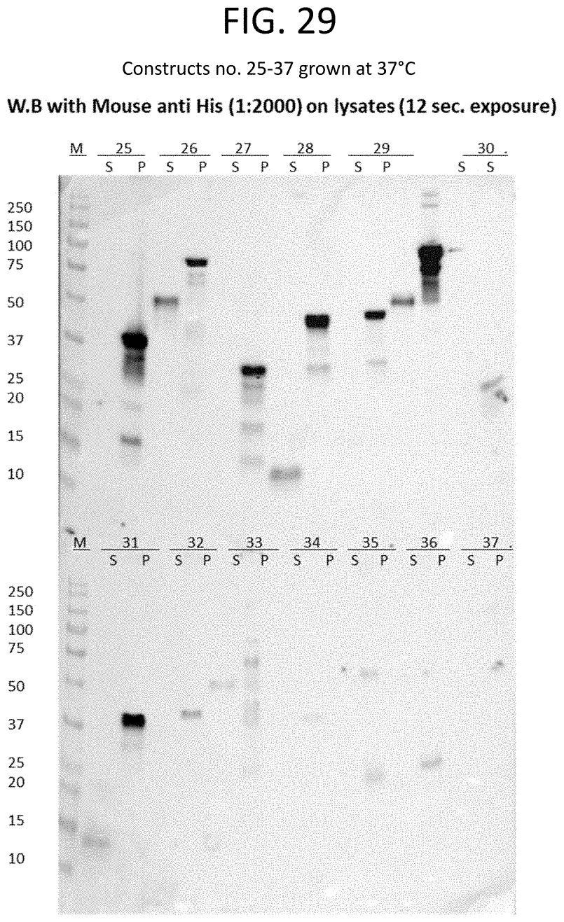

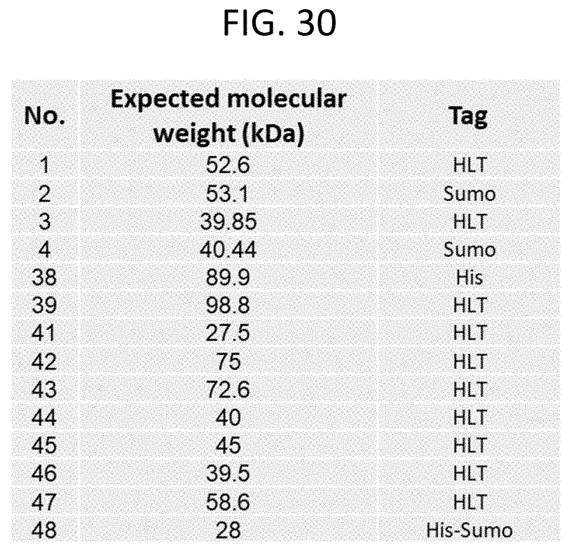

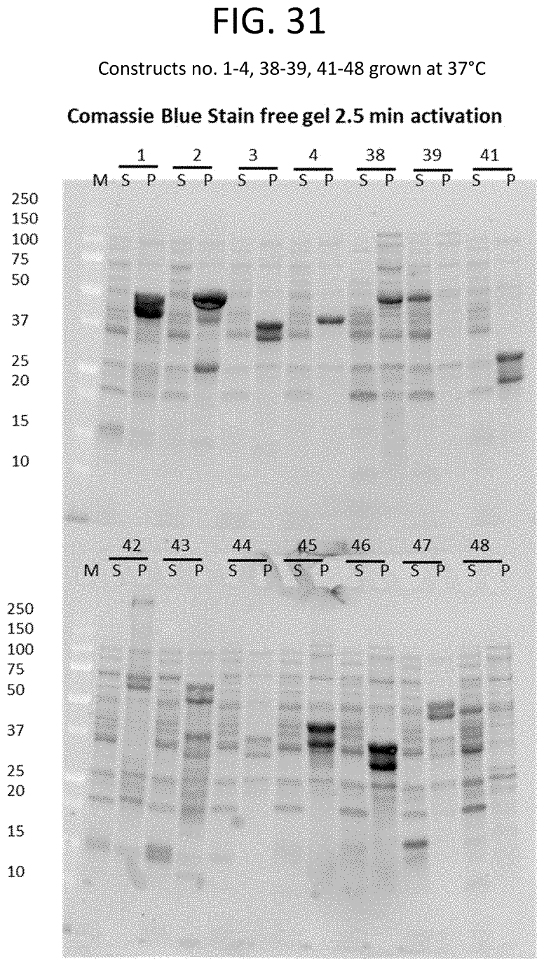

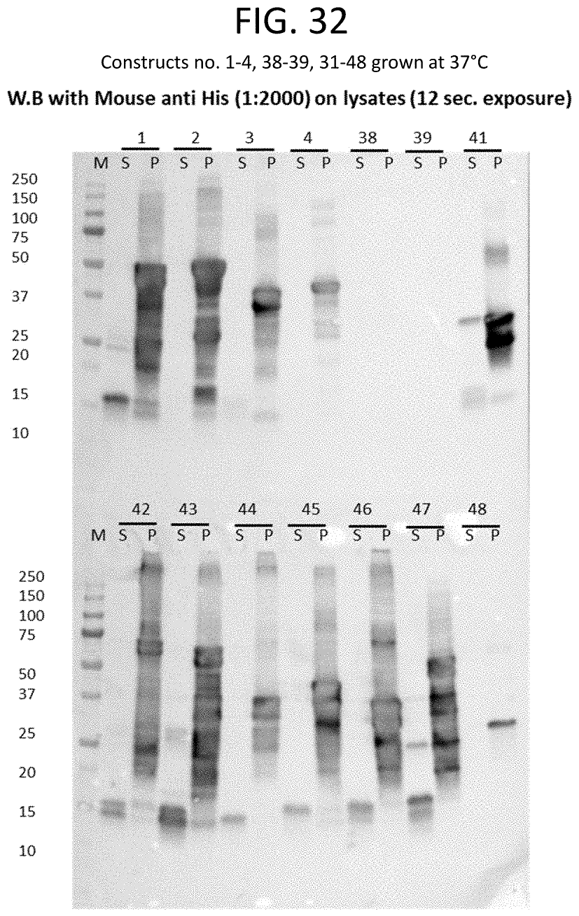

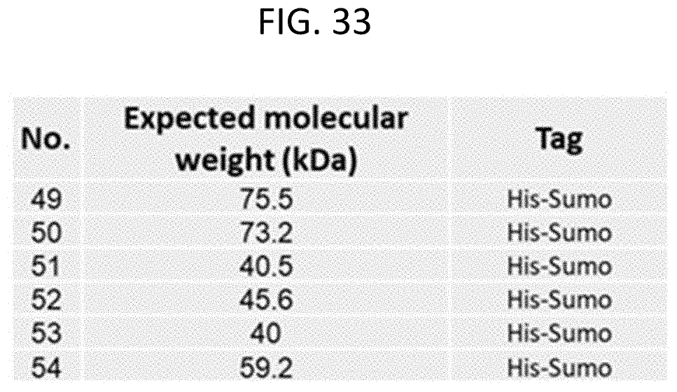

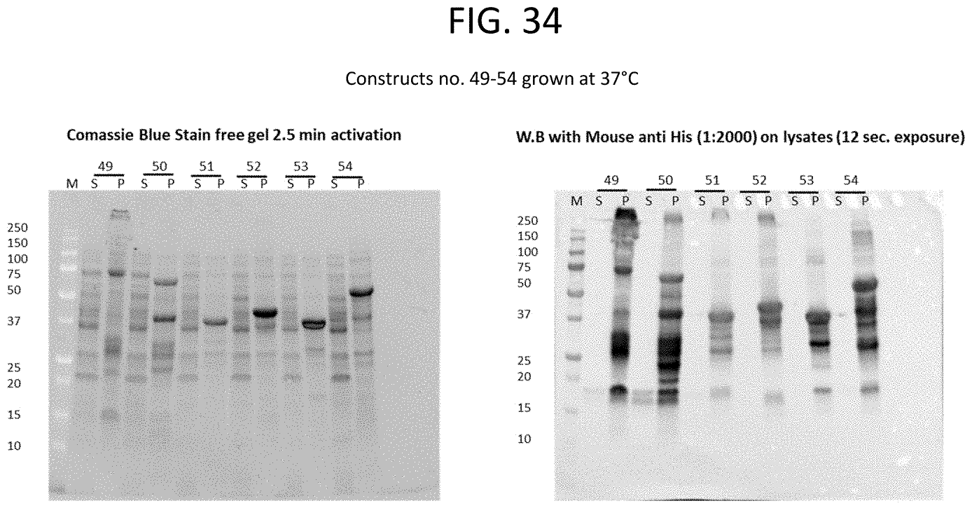

Proteins collected from culture supernatants and cell pellets were assessed using Coomassie Blue staining and immunoblotting. Proteins were separated using polyacrylamide gel electrophoresis. After gels were stained with Coomassie Blue and imaged, proteins were transferred onto PVDF membranes and detected via western blotting using anti-His antibodies. FIGS. 11-34 show gel staining and western blotting results for each of 54 constructs expressed in E. coli at either 22° C. (FIG. 11-21) or 37° C. (FIGS. 22-34), along with the expected molecular weight and specific one or more tags for each construct. Sequences of constructs labeled 1-54 are provided in SEQ ID NOs. 2310-2330. While constructs 1-54 each included an N-terminal His-tag for detection purposes, certain constructs also included at least one fusion protein attached directly to the N-terminus of the construct sequence, such as HLT, Sumo, or MBP. For constructs including a fusion protein, the His-tag was attached to the N-terminus of the fusion protein. Constructs as tested in this Example included the following fusion proteins: Construct 1: HLT; 2: Sumo; 3: HLT; 4: Sumo; 5: HLT; 6: Sumo; 7: HLT; 8: Sumo; 9: HLT; 10: Sumo; 11: no fusion protein; 12: HLT; 13: Sumo; 14: MBP; 15: no fusion protein; 16: HLT; 17: Sumo; 18: MBP; 19: no fusion protein; 20: HLT; 21: Sumo; 22: MBP; 23: no fusion protein; 24: HLT; 25: Sumo; 26: MBP; 27: no fusion protein; 28: HLT; 29: Sumo; 30: MBP; 31: no fusion protein; 32: HLT; 33: Sumo; 34: MBP; 35: no fusion protein; 36: HLT; 37: Sumo; 38: no fusion protein; 39: HLT; 40: Sumo; 41-47: HLT; 48-54: Sumo.

In each of FIGS. 11-34 showing Coomassie blue stained gels and/or western blots, “M” shows the marker lane denoting band molecular weights, “S” represents proteins collected from cell culture supernatants, and “P” represents proteins collected from cell pellets.

Table 8 provides a summary of the expression findings for each of the 54 constructs assessed. Column 2 of Table 8 describes where (in the cell pellet or in the culture supernatant/soluble fraction) a given expressed construct was detected. Western blotting detected protein products resulting from construct expression in E. coli primarily in cell pellets. Proteins were detected in culture supernatants for constructs 41-47, although at lower levels than in cell pellets for the same constructs. Construct 1, 3, 6, 9, 10, 11, 13, 16, 24, 27, 28, and 31 showed strong expression and were selected for further optimization. These constructs were again assessed for expression in E. coli, this time in two types of media, autoinduced media (AI) and Terrific Broth (TB). The “Constructs for optimization” column in Table 8 provides a qualitative assessment of expression (strong expression or weak expression) for each assessed construct, along with the optimal media (AI and/or TB) for expression of that construct in E. coli. Constructs were further selected for in vivo verification studies based on strong expression in the media optimization study. Because certain constructs differed only in their fusion proteins (MBP, Sump, or HLT), for a given group of constructs that otherwise shared sequence identity, only one fusion protein per construct was selected for the verification studies.

TABLE 8
Construct Expression Results

| | Expression (at 37° C. | | |
|---|---|---|---|
| | for constructs 1-40, | | Constructs |
| Construct | and at 22° C. or lower | Constructs for | for |
| no. | for constructs 41-54) | optimization | purification |
| 1 | Pellet | Strong expression | Verified |
| | | IB (AI) | |
| 2 | Pellet | | |
| 3 | Pellet | Strong expression | Verified |
| | | IB (AI) | |
| 4 | Pellet | | |
| 5 | Pellet | | |
| 6 | Pellet | Strong expression | Verified |
| | | IB (AI) | |
| 7 | Pellet | | |
| 8 | Pellet | | |
| 9 | Pellet | Strong expression | Verified |
| | | IB (AI) | |
| 10 | Pellet | Strong expression | ? |
| | | IB (AI) | |
| 11 | Pellet | Weak expression IB | |
| 12 | Pellet | | |
| 13 | Pellet | Strong expression | Verified |
| | | IB (AI) | |
| 14 | Pellet | | |
| 15 | Pellet | | |
| 16 | Pellet | Strong expression | Verified |
| | | IB (AI/TB) | |
| 17 | Pellet | | |
| 18 | Pellet | | |
| 19 | no expression | | |
| 20 | no expression | | |
| 21 | no expression | | |
| 22 | no expression | | |
| 23 | no expression | | |
| 24 | Pellet | Weak expression IB | |
| 25 | Pellet | | |
| 26 | Pellet | | |
| 27 | Pellet | Strong expression | Verified |
| | | IB (AI) | |
| 28 | Pellet | Strong expression | ? |
| | | IB (AI) | |
| 29 | Pellet | | |
| 30 | no expression | | |
| 31 | Pellet | Weak expression IB | |
| 32 | no expression | | |
| 33 | no expression | | |
| 34 | no expression | | |
| 35 | no expression | | |
| 36 | no expression | | |
| 37 | no expression | | |
| 38 | no expression | | |
| 39 | no expression | | |
| 40 | not tested? | | |
| 41 | Pellet (can be soluble) | | |
| 42 | Pellet (can be soluble) | | |
| 43 | Pellet (can be soluble) | | |
| 44 | Pellet (can be soluble) | | |
| 45 | Pellet (can be soluble) | | |
| 46 | Pellet (can be soluble) | | |
| 47 | Pellet (can be soluble) | | |
| 48 | Pellet | | |
| 49 | Pellet | | |
| 50 | Pellet | | |
| 51 | Pellet | | |
| 52 | Pellet | | |
| 53 | Pellet | | |
| 54 | Pellet | | |

(IB: inclusion bodies; AI: autoinduced media; TB: Terrific Broth)

### Example 4

### Composition Administration and Analysis In Vivo

This example describes in vivo validation studies aimed at assessing the ability of one or more compositions comprising a viral vector expressing one or more peptides of SEQ ID NOs. 2-2273 and/or one or more constructs of SEQ ID NOs. 2310-2330 to induce an immune response against ASFV in swine and to immunize (vaccinate) swine against ASFV.

The peptides expressed by the selected vector, a pseudorabies viral vector in this example, are selected from (1) SEQ ID NOs. 2-2273 based on the ability of the peptides to produce an immune response above a specified threshold as measured in porcine peripheral blood lymphocytes using an ELISpot assay as described in Example 2, and/or (2) SEQ ID NOs. 2310-2330 based on level of expression as described in Example 3.

The ten most promising candidate peptides are selected based on ELISpot assay results. Pseudorabies viral vectors are produced that express each peptide individually, and vaccine compositions comprising these vectors are also produced. In initial testing in swine, some of the ten compositions induce adequate humoral immune responses, as measured by liquid phase blocking ELISAs. A new pseudorabies viral vector is then produced that expresses each of the peptides of the compositions that induced the adequate humoral immune responses. Two compositions comprising the new vector are produced: one adjuvanted and one unadjuvanted, but otherwise comprising the same components.

Similarly, the five most promising candidate constructs of SEQ ID NOs. 2310-2330, are selected based on expression analyses, along with constructs 55 and 56. Pseudorabies viral vectors are produced that express each construct individually, and compositions comprising the new vectors are produced: one adjuvanted and one unadjuvanted, but otherwise comprising the same components.

The ability of the compositions to stimulate an immune response in swine and to provide protection against ASFV challenge is evaluated. For each viral vector produced, two groups of swine are vaccinated intramuscularly or intranasally. The first group receives the unadjuvanted composition comprising the viral vector and the second group receives the adjuvanted composition comprising the viral vector. One subset of vaccinated swine from each group is administered a second dose of the same composition at some interval following initial dosing, such as 28 days post (initial) vaccination (dpv). A second subset of vaccinated swine is administered a second dose at a different interval following initial dosing, such as 180 dpv. A third subset of vaccinated swine is administered a second dose at 28 dpv and a third dose 180 dpv. To assess the immune responses of treated swine, serum samples are collected from the swine prior to vaccination (day 0), as well as 4, 7, 14, 28, 56, 180, 208, 270, and 298 days following the first administration. Additionally, to study maternally-derived antibody (MDA) titers, serum samples are collected from piglets at 21 and 42 days of age that are born from vaccinated sows.

A subset of vaccinated swine from each group is challenged 50 days after the first vaccine administration using the ASFV China/2018/AnhuiXCGQ strain propagated in primary porcine alveolar macrophages and quantified using qPCR and hemadsorption assays. Serum samples are collected from challenged swine on the same schedule as for non-challenged (vaccinated only) swine.

Serum samples from all animals in this study are analyzed using liquid phase blocking ELISA for the detection of peptide-specific antibodies. IFN-γ is detectable in serum beginning 4 dpv. Swine vaccinated on days 0, 28, and 180 show the highest peptide-specific antibody titers 298 dpv, while the antibodies are undetectable by day 270 in swine administered the vaccine only once (on day 0). Swine administered the adjuvanted vaccine have higher peptide-specific antibody titers than swine administered the unadjuvanted vaccine. While piglets born from immunized sows show high passive antibody titers on day 21, titers have declined by day 42, suggesting that piglets born from immunize sows should receive boosters by about two months of age. In challenged animals, the ASFV genome and infectious virus are detectable at days 5 and 10 following challenge. Control (unvaccinated) pigs develop signs of acute ASF, while vaccinated animals develop no or only mild symptoms. The ASFV genome is detectable 60 days following challenge in vaccinated swine, although levels have declined significantly by day 60. Infectious virus is undetectable in challenged swine by day 35 following challenge. All vaccinated animals tolerate the compositions well, and no negative side effects attributable to the compositions are observed. The results of this study support the use of a viral vector expressing one or more ASFV-specific peptides in the development of a vaccine to protect swine against ASFV infection.

### Example 5

### Composition Administration and Analysis In Vivo

This example describes in vivo validation studies that were used to assess the ability of one or more compositions comprising a viral vector expressing one or more of the peptides of Appendix V and/or one or more of the peptides of Appendix VI to induce an immune response against ASFV in swine and to immunize swine against ASFV.

Peptides were chemically synthesized for use in this trial. The main objective of this trial was to evaluate cellular immune response following prime-boost vaccination using compositions comprising synthetic peptides with different adjuvants. Further, animal CD8 responses were evaluated, and animal immune responses using several approved adjuvants were compared.

### 1. Study Design

1. Three pregnant female pigs were located in the animal facility in separated cages. Approximately 30 newborn piglets were farrowed within the facility.

2. Three days post farrowing, piglets were administered with iron injection (for example, Ferraject 200, Eurovet Animal Health) in the right leg per each piglet.

3. Two weeks post farrowing, piglets were weighed, and the highest-weighted piglets were chosen for the experiment. Piglets will be divided into 5 groups, 3 piglets per group, with each group having 1 piglet from each mother to increase the breed variability.

4. Three weeks post farrowing. blood was collected from three piglets, which piglets were not included in the trial for ELISpot optimization.

5. Twelve (12) ear marked pigs 4 weeks old were vaccinated with the following vaccines.

Group 1: Three (3) pigs each were vaccinated with a composition comprising the 77 peptides of Appendix V in Emusigen P intramuscularly in the left leg, and with a composition comprising the 77 peptides of Appendix V in Carbigen+c-di-GMP intranasally.

Group 2: Three (3) pigs each were vaccinated with a composition comprising the 18 peptides of Appendix VI in Emusigen P intramuscularly in the left leg, and with a composition comprising the 18 peptides of Appendix VI in Carbigen+c-di-GMP intranasally.

Group 3: Three (3) pigs each were vaccinated with a composition comprising the 77 peptides of Appendix V in ISA 201+Quil-A+R848+TDB intramuscularly in the left leg, and with a composition comprising the 77 peptides of Appendix V in Carbigen+c-di-GMP+poly (I:C) intranasally.

Group 4: Three (3) pigs each were vaccinated with a composition comprising the 18 peptides of Appendix VI in ISA 201+Quil-A+R848+TDB intramuscularly in the left leg, and with a composition comprising the 18 peptides of Appendix VI in Carbigen+c-di-GMP+poly (I:C) intranasally.

Group 5 (Control pigs): Two (2) pigs were used as non-vaccinated controls.

TABLE 9
Trial groups

| Group | Formula | Route | Peptide mixture | # of pigs |
|---|---|---|---|---|
| 1 | Emulsigen P | Intra-muscular | 77 peptides | 3 |
| | Carbigen + c-di-GMP + | Intra-nasal | | |
| | poly (I:C) | | | |
| 2 | Emulsigen P | Intra-muscular | 18 peptides | 3 |
| | Carbigen + c-di-GMP + | Intra-nasal | | |
| | poly (I:C) | | | |
| 3 | ISA 201 + Quil-A + | Intra-muscular | 77 peptides | 3 |
| | R848 + TDB | | | |
| | Carbigen + c-di-GMP + | Intra-nasal | | |
| | poly (I:C) | | | |
| 4 | ISA 201 + Quil-A + | Intra-muscular | 18 peptides | 3 |
| | R848 + TDB | | | |
| | Carbigen + c-di-GMP + | Intra-nasal | | |
| | poly (I:C) | | | |
| 5 | Non injected group | Not applicable | Not applicable | 2 |

6. The second vaccination (boost) was given at 3 weeks following the first vaccination (same dosage per pig). Whole blood samples were collected during the trial at 34, 35, 55, 56, 62, and 63 days post first vaccination. Collection on days 62 and 63 is optional and was conducted as needed.

7. All bleedings were done into CPT tubes in 8 mL whole blood per tube. Three CPT tubes for groups 1 and group 3. Two CPT tubes for groups 2, 4 and 5.

TABLE 10
Trial Timeline

| Age | | | | | | | | | |
|---|---|---|---|---|---|---|---|---|---|
| (Weeks) | 3 W | 4 W | 7 W | 9 W | | 12 W | | 13 W (Optional) | |
| Trial | −1 | 1 | 21 | 34 | 35 | 55 | 56 | 62 | 63 |
| (Days) | | | | | | | | | |
| Activity 1 | | V | V | | | | | | |
| Activity 2 | *B | | | B | B | B | B | B | B |
| | 3 | | | Groups | Groups | Groups | Groups | Groups | Groups |
| | pigs | | | 1 + 3 | 2 + 4 + 5 | 1 + 3 | 2 + 4 + 5 | 1 + 3 | 2 + 4 + 5 |
| Activity 3 | | | | | | | E | | E |

Activity 1: V = vaccination
Activity 2: B = bleeding
Activity 3: E = Euthanasia
*Whole blood was taken from 3 pigs, which pigs were not included in the trial groups.

TABLE 11
Trial events

| Trial day | Action |
|---|---|
| −1 | Bleeding for ELISpot optimization from 3 |
| | piglets that were not included in the trial |
| 0 | 1^{st} vaccination (Prime) |
| 21 | 2^{nd} vaccination (Boost) |
| 34, 35, 55, 56 and 62 | Bleedings for PBMC isolation |
| (Optional), 63 (Optional) | |
| 63 | Completion (Pigs euthanize) |

### 2. Study Animals—Animal Selection and Identification

Three pregnant female pigs were located in the animal facility in separated cages. Approximately 30 newborn piglets were calved within the facility. Three days after calving, each piglet was administered with iron injection in the right leg. Fourteen (14) ear marked pigs 3 weeks old were vaccinated as shown in Table 9.

### 3. Materials

3.1 Adjuvants:

MONTANIDE ISA 201 VG: is a mineral oil-based adjuvant which has been developed for the formulation of Water-in-Oil-in-Water (W/O/W) emulsions. It is based on a specific enriched light mineral oil and a highly refined emulsifier obtained from mannitol and purified oleic acid from vegetable origin. MONTANIDE ISA 201 VG is free of animal origin ingredients. Vaccine formulations with MONTANIDE ISA 201 VG induce short- and long-term immunity. Compared to traditional double emulsions, MONTANIDE ISA 201 VG emulsions are stable, with low viscosity and are easy to inject.

Vaccine preparation:

To prepare 100 g of vaccine with a one-step process:

1. MONTANIDE ISA 201 VG 50 g

2. Aqueous antigenic medium 50 g

For preparation in volume, MONTANIDE ISA 201 VG density is about 0.83 at 20° C.

Each phase is heated to 31° C. before mixing. Stable preparations are obtained by mixing the aqueous medium into MONTANIDE ISA 201 VG under a low shear agitation (to maintain temperature above 30° C.). After formulation, emulsions are cooled down.

CARBIGEN™ and POLYGEN™ (Carbigen) are MVP's polymer-type adjuvants. Because of its muco-adhesive properties, CARBIGEN is particularly applicable for presenting inactivated antigens to mucosal membranes (e.g., intranasal). Intranasal vaccines incorporating inactivated antigens with CARBIGEN have been used successfully in horses, pigs, and small animals. It has also shown exceptional performance in adjuvanting PCV2 antigens.

Instruction for use:

1. With acid stable antigens, add 1-10% v/v of CARBIGEN to the antigen, mix well for 1-8 hours and raise pH carefully to approximately 7.0 with lON ^{NaOH.*} Mix an additional 12-24 hours. If necessary, readjust pH to between 6.8 and 7.2.

2. With acid labile antigens, add 10% v/v of CARBIGEN to a vessel equipped with a mixer. Adjust the pH of the CARBIGEN using lON NaOH to as low a pH as the antigen will tolerate without damage. * The lower the pH that the antigen can tolerate, the better will be the adjuvanting characteristics. When adjuvant is adjusted to the proper pH, add about 10% of the total antigen volume and mix for at least 30 minutes. The pH may drop. Readjust the pH and add the remainder of the antigen. Adjust the final pH to between 6.8 and 7.2. Mix at least an additional 12 hours (overnight) and readjust the pH, if necessary. Recheck the pH prior to filling. A small amount of NaCl or PBS may also be added to the antigen or to the CARBIGEN to reduce viscosity.

* Caution: Do not to raise the pH above 7.5. Addition of HCl or other acids to bring pH down, if too much NaOH is added, may decrease the effectiveness of the adjuvant.

EMULSIGEN®, MVP product, was used in the first vaccine that contained an oil-in-water adjuvant that was approved by USDA for both intramuscular and subcutaneous injection of pigs. Since that approval in 1982, it has been used globally in 45 countries and has a proven track record of being consistently safe and effective in all species of animals.

Instruction for use:

1. For most antigens, we recommend that EMULSIGEN-P be used at 10% to 20% (v/v).

2. EMULSIGEN-P should be gently mixed for up to 2 hours before adding to the antigen. During addition to the antigen, it is recommended that gentle mixing using standard equipment (e.g. Lightning mixer or magnetic stirrers) be continued for from 2-24 hours.

3. Continue gently mixing the product throughout filling to assure consistency.

4. Products containing EMULSIGEN-P may be administered intramuscularly or subcutaneously in a wide variety of animals.

5. It is normal for final vaccines to develop a creaming layer on top during storage. This does not adversely affect the antigenicity or immunogenicity. Simple inversion of the vials prior to injection is adequate to remix all components.

Quil-A adjuvant is a saponin adjuvant produced by GMP by Brenntag Biosector, a leader in the global vaccine adjuvants market, and purified by them through a proprietary process that ensures consistency and immunostimulatory potential. Quil-A adjuvant is used in a wide variety of veterinary vaccines, as well as in immunological research into human and veterinary applications. Quil-A adjuvant contains the water-extractable fraction of saponins from the South American tree, Quillaja saponaria Molina.

Preparation of Stock Solution (10 mg/ml)

1. Weigh 100 mg of Quil-A adjuvant. Place in a clean container. 2. Add 10 ml of distilled water to 100 mg of Quil-A adjuvant. 3. Mix using a magnetic stirrer until all the material has dissolved.

4. Immediately after dissolving the lyophilized powder, pass it through a 0.22-micron sterility filter into a sterile container under laminar air flow (Class A) in Class B surroundings.

5. After sterile filtration the Quil-A adjuvant solution should be stored frozen until use. Prepare aliquots to avoid repeated freeze-thaw cycles.

6. Due to the risk of alkaline hydrolysis, do not expose Quil-A adjuvant to a pH above 8.5.

TDB: Trehalose-6,6-dibehenate (TDB) is a non-toxic synthetic analogue of the mycobacterial cell wall component trehalose 6,6′ dimycolate (TDM, also known as cord factor).

### Preparation of Stock Suspension (1mg/mL)

1. Add 1004, DMSO to 1mg TBD VacciGrade, heat at 60° C. (approx.15-30 seconds) and vortex.

2. Once resuspended, immediately add 9004, sterile physiological water (provided) or phosphate buffered saline (PBS without Ca2+and Mg2+), heat for 10-15 minutes at 60° C. and homogenize by vortexing for 30 seconds.

3. Store at 4 ° C. or prepare dilutions using buffered solution for immediate use. Resuspended product can be stored at 4 ° C. for 6 months. Prior to each use, bring suspension to room temperature and homogenize by vortexing for 30 seconds.

R848 (resiquimod): a small molecular weight imidazoquinoline compound, is an immune response modifier with potent antiviral and antitumor activities. R848 is being evaluated as an adjuvant in FDA-approved clinical vaccine trials.

### Preparation of Sterile Stock Solution (1 mg/mL)

1. Add 5 mL endotoxin-free physiological water to the 5 mg R848 VacciGrade vial to obtain a solution at 1 mg/mL.

2. Mix the solution by pipetting up and down.

c-di-GMP: Cyclic diguanylate monophosphate (c-di-GMP) is an intracellular signaling molecule produced by bacteria. Administration of c-di-GMP can induce a strong immune response in vitro and in vivo.

### Preparation of Sterile Stock Solution (1 mg/mL)

1. Add 1 mL endotoxin-free physiological water to the 1 mg c-di-GMP VacciGrade vial to obtain a solution at 1 mg/mL.

2. Mix the solution by pipetting up and down.

Poly (I:C) HMW: Polyinosinic-polycytidylic acid is a synthetic analog of double stranded RNA (dsRNA), a molecular pattern associated with viral infection. Both natural and synthetic dsRNA are known to induce type 1 interferon (INF) and other cytokines production. Poly (I:C) is recognized by TLR3.

Preparation of Sterile Stock Solution (1 mg/mL)

1. Add 10 mL endotoxin-free physiological water to the 10 mg Poly (I:C) vial to obtain a solution at 1 mg/mL.

2. Mix the solution by pipetting up and down.

3. Heat the mixture for 10 minutes at 65-70° C. Allow the solution to cool for 1 hour at room temperature to ensure proper annealing.

3.2 Peptides:

77 ASFV positive peptides identified through ELISpot screenings were chemically synthesized to at least 70% purity by JPT (Berlin). Among these 77 positive peptides (each of which produced greater than or equal to 20 spots in the ELISpot assays), 18 peptides were defined as “top” positives with respect to their ELISpot scores (FIGS. 1-3). In this trial, two peptide mixtures were tested: the first mixture contained all 77 peptides; the second mixture contained only the 18 “top” peptides.

The stock solution for each peptide was produced at a concentration of 5 mg/mL. Every peptide was dissolved in 1 mL water for injection, except peptide 554, which was dissolved in 100 DMSO plus 900 μL water for injection. Two stock plates were prepared and frozen at −70° C. until vaccine preparation. The work was done in sterile conditions.

### 4. Vaccine Preparations:

During the study vaccination was performed twice per Group (see Tables 10 and 11 for vaccination time points) according to the vaccination instruction per each group. In addition, the vaccine preparation instructions are described per each vaccination event.

### Group 1-77 Peptides in Emulsigen P Vaccine

Intramuscular vaccine: 125 μg of each peptide was mixed with Emulsigen P adjuvant for a total volume of 5 mL (5 doses). The final dose contained 25 μg from each peptide in an injection volume of 1 mL. 1 mL of the vaccine was injected into each pig's left leg.

TABLE 12
Group 1-Vaccine Preparation (intramuscular dose)

| | | Concentration | Stock | Volume |
|---|---|---|---|---|
| IM vaccine | Substance | per dose | solution | (mL) |
| 1 | 77xPeptide | 25 μg (each) | 5 mg/mL | 25 μL (each) |
| | | | | 1.925 mL |
| | | | | (Total) |
| 2 | Emulsigen P | 12% (v/v) | Ready to use | 0.6 |
| 3 | PBS without | NA | Ready to use | 2.475 |
| | Ca and Mg | | (x1) | |
| Final vol. | | | | 5 |
| (mL) | | | | |

### Group 2-18 Peptides in Emulsigen P Vaccine

Intramuscular vaccine: 125 μg of each peptide was mixed with Emulsigen P adjuvant for a total volume of 5 mL (5 doses). The final dose contained 25 μg of each peptide in an injection volume of 1 mL. 1 mL of the vaccine was injected into each pig's left leg.

TABLE 13
Group 2-Vaccine Preparation (intramuscular dose)

| IM | | Concentration | Stock | Volume |
|---|---|---|---|---|
| vaccine | Substance | per dose | solution | (mL) |
| 1 | 18xPeptide | 25 μg | 5 mg/mL | 25 μL (each) |
| | | | | 0.45 mL |
| | | | | (Total) |
| 2 | Emulsigen P | 12% (v/v) | Ready to use | 0.6 |
| 3 | PBS without Ca | NA | Ready to use | 3.95 |
| | and Mg | | (x1) | |
| Final vol. | | | | 5 |
| (mL) | | | | |

### Group 3-77 Peptides in ISA 201 Plus Quil-A Plus R848 Plus TDB Vaccine

Intramuscular vaccine: 125 μg of each peptide were mixed with ISA 201 (50%, w/w), 150 μg Quil-A, 50 μg R848, and 50 μg TDB for a total volume of 5 mL (5 doses). The final dose contained 25 μg from each peptide in an injection volume of 1 mL. 1 mL of the vaccine was injected into each pig's left leg.

TABLE 14
Group 3 Vaccine Preparation (intramuscular dose)

| | | Concentration | Stock | Volume |
|---|---|---|---|---|
| IM vaccine | Substance | per dose | solution | (mL) |
| 1 | 77xPeptide | 25 μg | 5 mg/mL | 25 μL (each) |
| | | | | 1.925 mL |
| | | | | (Total) |
| 2 | ISA 201 | 50/50 (w/w) | Ready to use | 2.5 |
| 3 | Ouil-A | 150 μg | 10 mg/mL | 0.075 |
| 4 | R848 | 50 μg | 1 mg/mL | 0.25 |
| 5 | TDB | 50 μg | 1 mg/mL | 0.25 |
| Final vol. (mL) | | | | 5 |

### Group 4-18 peptides in ISA 201 Plus Quil-A Plus R848 plus TDB vaccine

Intramuscular vaccine: 125 μg of each peptide was mixed with ISA 201 (50%, w/w), 150 Quil-A, 50 μg R848, and 50 μg TDB for a total volume of 5 mL (5 doses). The final dose contained 25 μg of each peptide in an injection volume of 1 mL. 1 mL of the vaccine was injected into each pig's left leg.

TABLE 15
Group 4 Vaccine Preparation (intramuscular dose)

| | | Concentration | Stock | Volume |
|---|---|---|---|---|
| IM vaccine | Substance | per dose | Solution | (mL) |
| 1 | 18xPeptide | 25 μg | 5 mg/mL | 25 μL (each) |
| | | | | 0.45 mL |
| | | | | (Total) |
| 2 | ISA 201 | 50/50 (w/w) | Ready to use | 2.5 |
| 3 | Ouil-A | 150 μg | 10 mg/mL | 0.075 |
| 4 | R848 | 50 μg | 1 mg/mL | 0.25 |
| 5 | TDB | 50 μg | 1 mg/mL | 0.25 |
| 6 | PBS without | NA | Ready to use | 1.475 |
| | Ca and Mg | | (x1) | |
| Final vol. | | | | 5 |
| (mL) | | | | |

### Groups 1 and 3-77 Peptides in Carbigen Plus C-Di-GMP Vaccine

Intranasal vaccine: 120 μg of each peptide was mixed with 10% (v/v) Carbigen, 50 μg c-di-GMP, and 50 μg poly (I:C) for a total volume of 8 mL (8 doses). The final dose contained 15 μg of each peptide in an injection volume of 1 mL. 0.5 mL of the vaccine was administered into each pig's nostril (for a total of 1.0 mL per pig) using MAD Nasal Drug Delivery Device (Teleflex).

TABLE 16
Groups 1 and 3 Vaccine Preparation (intranasal dose)

| Intranasal (IN) | | Concentration | Stock | Volume |
|---|---|---|---|---|
| vaccine | Substance | per dose | solution | (mL) |
| 1 | 77xPeptide | 15 μg | 5 mg/mL | 24 μL (each) |
| | | | | 1.848 mL |
| | | | | (Total) |
| 2 | Carbigen | 10% (v/v) | Ready to use | 0.8 |
| 3 | c-di-GMP | 50 μg | 1 mg/mL | 0.4 |
| 4 | Poly (I:C) | 50 μg | 1 mg/mL | 0.4 |
| 5 | PBS without | NA | Ready to use | 4.552 |
| | Ca and Mg | | (x1) | |
| Final vol. (mL) | | | | 8 |

### Groups 2 and 4-18 Peptides in Carbigen Plus c-di-GMP Vaccine

Intranasal vaccine: 120 μg of each peptide was mixed with 10% (v/v) Carbigen, 50 μg c-di-GMP, and 50 μg poly (I:C) for a total volume of 8 mL (8 doses). The final dose contained 15 μg of each peptide in an injection volume of 1 mL. 0.5 mL of the vaccine was administered into each pig's nostril using MAD Nasal Drug Delivery Device (Teleflex).

TABLE 17
Groups 2 and 4 Vaccine Preparation (intranasal dose)

| IN | | Concentration | | Stock | | Volume | |
|---|---|---|---|---|---|---|---|
| vaccine | Substance | per dose | | Solution | | (mL) | |
| 1 | 18xPeptide | 15 | μg | 5 | mg/mL | 24 | μL |

(each)

| | |
|---|---|
| 0.432 | mL |

| | | | | | | (Total) |
|---|---|---|---|---|---|---|
| 2 | Carbigen | 10% (v/v) | | Ready to use | | 0.8 |
| 3 | c-di-GMP | 50 | μg | 1 | mg/mL | 0.4 |
| 4 | Poly I:C | 50 | μg | 1 | mg/mL | 0.4 |

| | | | | |
|---|---|---|---|---|
| 5 | PBS without | NA | Ready to use | 5.968 |
| | Ca and Mg | | (x1) | |
| Final vol. | | | | 8 |
| (mL) | | | | |

5. Bleeding procedure:

Whole blood (8 mL) was collected into CPT tubes from animals from groups 1-4 at the bleeding time points (post vaccination) shown in Table 10.

1. Work was performed under aseptic conditions as much as possible. Prepare: Alcohol 70%, gauzes, Vacutainer (20G), CPT tubes (Vol: 8 mL) at room temperature.

2. Restrain animal with snare, securely contained against a wall or corner; alternatively, swine can be placed in a sling, smaller pigs can be held or placed in v-trough.

3. Clean as needed to remove superficial dirt and debris. Locate jugular furrow and align with point of the shoulder and point of the manubrium. With bevel up, insert needle perpendicular to the skin.

4. If using vacutainer, once needle inserted, stabilize needle and push the vacutainer tube into hub. If you have hit the vein, blood will flow freely into tube. Multiple tubes can be filled by removing filled tube and replacing with fresh tube.

5. If you have missed the vein, you can carefully reposition needle, with vacutainer attached, until vessel penetrated. The vessel is fairly deep and may roll away from needle. Typically, no more than two to three attempts should be made at a time to minimize distress to the animal and potential damage to the vein.

6. Alternately, you can use needle and syringe. Break the seal on the syringe by gently pulling back before using.

7. Clear air, and with needle attached to syringe, insert needle firmly at a 90° angle, and aspirate syringe to confirm insertion and collect blood.

8. Once collection is complete, remove vacutainer tube. Then, applying pressure over injection site, remove needle. Dispose of needle in approved Sharps container.

9. Keep the blood contained in CPT tubes at room temperature. Blood samples will be collected within one hour by IIBR or Phibro members.

10. In order to ensure adequate hemostasis, apply pressure for 30 to 60 seconds.

### 6. Criteria for Inclusion/Exclusion and Post Inclusion Removal Criteria:

Inclusion: clinical and behavioral healthy animals without any signs of disease. Animals started the experiment after a minimum one week of acclimation. During the acclimation period, animals underwent inspection, and only if they continued to look healthy did they start the experiment.

Exclusion: Animals with extensive wounding and/or illness that was not connected to the experiment. Illness due to the vaccination procedure (such as anorexia). Adverse events reporting and recording: Pigs underwent inspection twice a day. Adverse events were documented and noticed to the study director.

### 7. Animal Management and Housing:

The health statuses of the animals used in the study was determined before entering the acclimation period, one week before the study's start point. Only animals in good health were acclimatized to laboratory conditions for 7 days prior to study initiation. Pigs were kept in group cages (according to the experimental groups).

Animal handling was performed according to guidelines of the National Institute of Health (NIH) and the Israeli Council for Experiments on Animals. Animals were housed within a limited access Large Animal unit (Biotech Farm Site) in concrete floor holding pens. Holding pens were cleaned once daily, six days per week.

Animals were provided with commercially available piglet diet, medical pre-starter for piglets (Kefar yeoshua' feedmil, Kefar yeoshua', Israel), at approximately 2-4% of a given pig's body weight per day, twice a day, and pigs were allowed free access to drinking water supplied by automated watering valves. Environmental conditions were set to maintain temperature at 24±6° C. with a relative humidity (RH) of about 30-70% and a 12-hr light/12-hr dark cycle. RH and temperature were recorded daily.

### 8. Safety of Study Personnel:

The procedures that were used in this study are considered of low risk to the operators. All procedures were performed with all necessary protective equipment. Face masks were used in order to prevent any spill off penetration.

Disposal of study products: According to Biotech farm approved procedures.
Disposal of study animals: According to Biotech farm approved procedures.

### 9. Assessment of Vaccination:

At several time points post vaccination, blood was collected into CPT tubes and peripheral blood mononuclear cells (PBMCs) were separated for measuring cellular immune response. Cells (2.5*10⁵, 5*10⁵, 1*10⁶per well) were incubated with each peptide. The positive control was concanavalin A (ConA) and the negative control was medium only. ELISpot assays were performed using CTL or the MabTech IFNγ ELISPOT kit.

In view of the many possible embodiments to which the principles of the disclosed invention may be applied, a person of ordinary skill in the art will recognize that the illustrated embodiments are only preferred examples of the invention and should not be taken as limiting the scope of the invention. Rather, the scope of the invention is defined by the following claims. We therefore claim as our invention all that comes within the scope and spirit of these claims.

APPENDIX I

| | | Amino Acid |
|---|---|---|
| | SEQ ID NO. | Sequence |
| 1 | 2 | NEMDIVQIFY |
| 2 | 3 | EMDIVQIFY |
| 3 | 7 | RAMVTSVKNFY |
| 4 | 11 | SSNVSLLSL |
| 5 | 17 | ANANRAMLI |
| 6 | 18 | KTLADIYGY |
| 7 | 21 | YANHCRFCW |
| 8 | 57 | RYTQIYKYPLI |
| 9 | 67 | YMENCKFCW |
| 10 | 69 | KSMPLIVENSY |
| 11 | 70 | CTYAKSCDF |
| 12 | 94 | NIDEVHHAY |
| 13 | 95 | RALERLISF |
| 14 | 97 | STYKNTESF |
| 15 | 98 | STYKNTESFY |
| 16 | 99 | KNTESFYPF |
| 17 | 100 | KMIKNTYVL |
| 18 | 102 | NVDEIHHAYF |
| 19 | 103 | SNVHFCISL |
| 20 | 109 | KNYSLSTLY |
| 21 | 110 | YSLSTLYCIF |
| 22 | 113 | RSDIDHMYAF |
| 23 | 124 | FEIYFARLY |
| 24 | 138 | LYVYSKTFYRK |
| 25 | 139 | LYVYSKTFY |
| 26 | 147 | SKTFYRKSWYW |
| 27 | 149 | KTFYRKSWY |
| 28 | 154 | TFYRKSWYW |
| 29 | 159 | RKSWYWFCIF |
| 30 | 161 | RKSWYWFCIFM |
| 31 | 162 | KSWYWFCIF |
| 32 | 163 | KSWYWFCIFM |
| 33 | 169 | KMLSYGMEW |
| 34 | 171 | KVRTFVCCY |
| 35 | 172 | YTLGGTASL |
| 36 | 179 | KLKRAISFFY |
| 37 | 186 | LLSDNPLFL |
| 38 | 187 | TLDNISFNEM |
| 39 | 188 | TLDNISFNEML |
| 40 | 189 | DNISFNEML |
| 41 | 191 | ISFNEMLTR |
| 42 | 195 | SFNEMLTRYW |
| 43 | 198 | NEMLTRYWY |
| 44 | 201 | EMLTRYWYSM |
| 45 | 202 | EMLTRYWYS |
| 46 | 205 | LTRYWYSMAI |
| 47 | 231 | AARQQIAVY |
| 48 | 234 | KTLPSIQNL |
| 49 | 241 | MNFFCNIKL |
| 50 | 247 | YHQKKIWTPY |
| 51 | 251 | QAMFHSIQF |
| 52 | 253 | AMFHSIQFY |
| 53 | 257 | SAMLACVRFY |
| 54 | 266 | MGANINQAM |
| 55 | 269 | LAWEGNLYY |
| 56 | 270 | YSKFRVLLY |
| 57 | 274 | ATYNHRKILIY |
| 58 | 275 | KISHYVATY |
| 59 | 278 | KTDLLNNEF |
| 60 | 279 | SLSTLLLKY |
| 61 | 280 | YAIAIRYNL |
| 62 | 283 | NVFDLHEAY |
| 63 | 287 | YTDLNEWRL |
| 64 | 293 | KSCAGVLLGY |
| 65 | 294 | ALRHNFTKAI |
| 66 | 297 | RHNFTKAIHY |
| 67 | 309 | TKAIHYFYK |
| 68 | 321 | KRHKNHLYWR |
| 69 | 328 | ASLDYGMNL |
| 70 | 329 | NNNTLNMFF |
| 71 | 330 | YMYNLSNIF |
| 72 | 333 | RAYLHETLF |
| 73 | 335 | TMYSLGYIF |
| 74 | 343 | STCSLKCLF |
| 75 | 345 | VSIKGLLPF |
| 76 | 357 | HVIQRLGLY |
| 77 | 370 | QIQDWHILL |
| 78 | 371 | MAIDNGLLPF |
| 79 | 375 | KQIVHTIKY |
| 80 | 379 | KHTLHLLGL |
| 81 | 385 | RTENYNLVCEY |
| 82 | 386 | HSQIQDWHVL |
| 83 | 389 | QIQDWHVLL |
| 84 | 425 | KTLNLLLSY |
| 85 | 429 | STLVIRLLL |
| 86 | 435 | STYFQVKEF |
| 87 | 437 | MQDFSISPEKF |
| 88 | 447 | IIHTIYQSY |
| 89 | 462 | IMQAYALEY |
| 90 | 463 | MQAYALEYAM |
| 91 | 467 | QAYALEYAMY |
| 92 | 469 | RQYDLIQKY |
| 93 | 471 | VTCTFQCLF |
| 94 | 477 | STYTEIVKY |
| 95 | 478 | HNITGYTYL |
| 96 | 481 | AVHNATCLF |
| 97 | 517 | KMIDSYNDY |
| 98 | 527 | LANAFIPPY |
| 99 | 554 | VLIEFLTGFF |
| 100 | 555 | LIEFLTGFFY |
| 101 | 557 | EFLTGFFYLY |
| 102 | 559 | TGFFYLYGK |
| 103 | 563 | RLFSISKVM |
| 104 | 569 | MDMICLDYY |
| 105 | 570 | YTIIPAPLAM |
| 106 | 573 | LAMMLAARL |
| 107 | 608 | KHDARMLINY |
| 108 | 609 | ARMLINYCV |
| 109 | 619 | HIRNGNLTLF |
| 110 | 621 | KTDPWIVNR |
| 111 | 625 | GRIDFLKFLF |
| 112 | 633 | KAAIRGRSL |
| 113 | 646 | GADPTQKDY |
| 114 | 647 | HRGFTAWDW |
| 115 | 651 | FTAWDWAVF |
| 116 | 655 | SLNHDYQNL |
| 117 | 661 | GGLRKSPKL |
| 118 | 662 | LRKSPKLLL |
| 119 | 665 | AVNIMSMKNF |
| 120 | 675 | LSMKNRELF |
| 121 | 687 | YINDISEHEL |
| 122 | 701 | ESTVITTAY |
| 123 | 703 | TVITTAYNF |
| 124 | 711 | FLAFSLHSDMY |
| 125 | 713 | LAFSLHSDMY |
| 126 | 724 | SDMYSVIFNI |
| 127 | 725 | DMYSVIFNIKY |
| 128 | 726 | DMYSVIFNI |
| 129 | 735 | VIFNIKYFLSK |
| 130 | 746 | QMDKLGFLL |
| 131 | 750 | TNFFTLHEL |
| 132 | 756 | CFMMQCKSIY |
| 133 | 762 | SSFSKAVRL |
| 134 | 769 | AMDEAIHAALY |
| 135 | 770 | AAMQGLRNGY |
| 136 | 771 | AMQGLRNGY |
| 137 | 784 | SKQASISSIL |
| 138 | 788 | KQASISSI |
| 139 | 790 | ASISSILNF |
| 140 | 810 | FFFYIMEYF |
| 141 | 815 | IMDVFYETY |
| 142 | 816 | YSLPYNINL |
| 143 | 818 | RTSPSYCEI |
| 144 | 819 | GTNNFVETY |
| 145 | 823 | RTYNILQRF |
| 146 | 825 | SHFNNVSYYW |
| 147 | 826 | NNVSYYWGL |
| 148 | 827 | QTISNHQLSF |
| 149 | 842 | SSMHSGMLYK |
| 150 | 848 | FLKKNIYLY |
| 151 | 849 | HSKALATLLY |
| 152 | 860 | RFNTLHIHY |
| 153 | 863 | MMRRVHASY |
| 154 | 865 | RVHASYPGY |
| 155 | 869 | CTQPARVTY |
| 156 | 872 | RVDMNRFFQFY |
| 157 | 880 | KTVEPTNFL |
| 158 | 896 | ITNKIYMFF |
| 159 | 908 | VGYNNVCYYF |
| 160 | 917 | ESNYWVNYSL |
| 161 | 918 | SNYWVNYSL |
| 162 | 920 | SVLLRDSGYY |
| 163 | 921 | SVLLRDSGY |
| 164 | 923 | KKQKHVSLLY |
| 165 | 925 | KQKHVSLLY |
| 166 | 926 | KQKHVSLLYI |
| 167 | 931 | VSFNKTIIL |
| 168 | 934 | VSWNFFNNSF |
| 169 | 954 | ISTSNETTL |
| 170 | 955 | STSNETTLI |
| 171 | 960 | TTLINCTYL |
| 172 | 962 | TLINCTYLTL |
| 173 | 963 | LINCTYLTL |
| 174 | 971 | LTLSSNYFYTF |
| 175 | 972 | LTLSSNYFYT |
| 176 | 986 | SNYFYTFF |
| 177 | 991 | YFYTFFKLY |
| 178 | 1000 | FKLYYIPLSI |
| 179 | 1006 | LLPKPYSRY |
| 180 | 1009 | SRYQYNTPI |
| 181 | 1010 | SRYQYNTPIYY |
| 182 | 1013 | RYQYNTPIY |
| 183 | 1026 | GSFSPETLGY |
| 184 | 1028 | FTHQYIELY |
| 185 | 1035 | LLYDLYRAGY |
| 186 | 1047 | ASLEFNTFY |
| 187 | 1048 | SLEFNTFYAF |
| 188 | 1049 | AVIEAIGAM |
| 189 | 1064 | KTRGTRLFF |
| 190 | 1065 | KTLKTVYPEY |
| 191 | 1090 | LFPQYISYY |
| 192 | 1091 | FPQYISYY |
| 193 | 1092 | FPQYISYYTKY |
| 194 | 1094 | FPQYISYYTK |
| 195 | 1101 | LIPKHLWSY |
| 196 | 1102 | WIRNNFSISY |
| 197 | 1106 | RTIPVAWDRF |
| 198 | 1107 | MTSLLKTDF |
| 199 | 1118 | FTRFANTSPF |
| 200 | 1129 | ITSNVLTTF |
| 201 | 1139 | SILAEYVYSY |
| 202 | 1141 | YSYNGMLEHY |
| 203 | 1156 | YGVETHWPLY |
| 204 | 1184 | SSIPKNKLF |
| 205 | 1187 | VSNILHSVF |
| 206 | 1193 | MLDSFYKYF |
| 207 | 1194 | ITTEKMLPF |
| 208 | 1196 | KEMQDYSLTFL |
| 209 | 1202 | MQDYSLTFLLK |
| 210 | 1203 | MQDYSLTF |
| 211 | 1204 | QDYSLTFLLK |
| 212 | 1210 | YSLTFLLK |
| 213 | 1227 | SSYNRSLLH |
| 214 | 1228 | RSSTSKSSY |
| 215 | 1231 | KTFNQSGLF |
| 216 | 1239 | RLIMTSFIGY |
| 217 | 1248 | KTLISEMMHY |
| 218 | 1264 | INRNYYPYY |
| 219 | 1265 | INRNYYPYYI |
| 220 | 1276 | NYYPYYIYK |
| 221 | 1277 | NYYPYYIY |
| 222 | 1278 | YPYYIYKIF |
| 223 | 1279 | YIYKIFDAI |
| 224 | 1285 | HLVHFNAHF |
| 225 | 1286 | VHFNAHFKPY |
| 226 | 1287 | HFNAHFKPY |
| 227 | 1288 | KPYVPVGFEY |
| 228 | 1295 | HGQLQTFPR |
| 229 | 1296 | QLQTFPRNGY |
| 230 | 1302 | TKNAYRNLVY |
| 231 | 1318 | ITDATYLDI |
| 232 | 1319 | YLDIRRNVHY |
| 233 | 1323 | AIPSVSIPF |
| 234 | 1329 | SRRNIRFKPW |
| 235 | 1338 | FVTPEIHNLF |
| 236 | 1340 | VTPEIHNLF |
| 237 | 1345 | KLMSALKWPI |
| 238 | 1347 | LMSALKWPIEY |
| 239 | 1348 | MSALKWPIEY |
| 240 | 1366 | FCSSYIPFHY |
| 241 | 1368 | KTPDDPGAMM |
| 242 | 1369 | GAMMITFAL |
| 243 | 1370 | FALKPREEY |
| 244 | 1372 | VSRAREFYI |
| 245 | 1375 | EFYISWDTDY |
| 246 | 1377 | YISWDTDYV |
| 247 | 1378 | VVSASAINF |
| 248 | 1379 | VSASAINFL |
| 249 | 1382 | SASAINFLLL |
| 250 | 1385 | LLQNGSAVL |
| 251 | 1388 | ATSHVATSY |
| 252 | 1389 | QQMLTRHIY |
| 253 | 1390 | NLAGITTLM |
| 254 | 1394 | KTVPKFVPTY |
| 255 | 1399 | KESAETIYTF |
| 256 | 1400 | ESAETIYTF |
| 257 | 1413 | SSMSVSTFW |
| 258 | 1415 | SMSVSTFWPY |
| 259 | 1426 | KAANTPQYY |
| 260 | 1432 | QQNKANKAF |
| 261 | 1436 | KANKAFYINH |
| 262 | 1437 | KANKAFYI |
| 263 | 1438 | KANKAFYINHL |
| 264 | 1441 | NKAFYINHLY |
| 265 | 1448 | AFYINHLYK |
| 266 | 1452 | YINHLYKFL |
| 267 | 1454 | INHLYKFLLI |
| 268 | 1460 | YTLLSPLQSY |
| 269 | 1461 | LLSPLQSYTY |
| 270 | 1468 | AADDTTCYY |
| 271 | 1469 | KSSEWTTIL |
| 272 | 1470 | MSLFWHQKL |
| 273 | 1471 | KVDLPYHLM |
| 274 | 1472 | GILSYTSLY |
| 275 | 1480 | GQYNLKLVY |
| 276 | 1482 | KTIKHYEQL |
| 277 | 1483 | GTSYLRMAY |
| 278 | 1484 | IAHVNTPNF |
| 279 | 1485 | KNLPIDILFY |
| 280 | 1488 | HLQAFLDSY |
| 281 | 1491 | IADAINQEF |
| 282 | 1492 | ASICRQIVLY |
| 283 | 1499 | FLNKSTQAY |
| 284 | 1500 | ALDLSLIGFY |
| 285 | 1501 | KTDPNFKNLY |
| 286 | 1503 | NQAINTFMYY |
| 287 | 1507 | RALEGLDLY |
| 288 | 1508 | TLAQVFESF |
| 289 | 1509 | FTDNAPAGHYY |
| 290 | 1510 | RSLSNFQAL |
| 291 | 1511 | QIYKTLLEY |
| 292 | 1512 | ETEDVFFTF |
| 293 | 1514 | RLAEFYQKL |
| 294 | 1517 | RTMNDFGMM |
| 295 | 1518 | FGMMNQTNY |
| 296 | 1523 | SLMADTKYF |
| 297 | 1528 | RVFSRLVFY |
| 298 | 1531 | FSQAVMEMGY |
| 299 | 1541 | RSIPLANIY |
| 300 | 1543 | GSLYPTQFDY |
| 301 | 1544 | SLYPTQFDY |
| 302 | 1556 | VVFHAGSLY |
| 303 | 1557 | HAGSLYNWF |
| 304 | 1564 | SARIYAGQGY |
| 305 | 1566 | QAQEEWNMIL |
| 306 | 1567 | AQEEWNMIL |
| 307 | 1571 | EQYGKAPDF |
| 308 | 1573 | IRAHNFIQTI |
| 309 | 1574 | IRAHNFIQTIY |
| 310 | 1576 | RAHNFIQTIY |
| 311 | 1577 | AHNFIQTIY |
| 312 | 1580 | MKQFCKISVW |
| 313 | 1592 | KTLESLILPF |
| 314 | 1601 | IMESGSMPL |
| 315 | 1619 | DFDPLVTFY |
| 316 | 1627 | SAMLEFKKF |
| 317 | 1628 | SAMLEFKKFF |
| 318 | 1630 | FTQITRQTF |
| 319 | 1631 | TQITRQTFM |
| 320 | 1633 | IADSATKEV |
| 321 | 1648 | TLMDQPTY |
| 322 | 1649 | RNLRFSRPG |
| 323 | 1651 | RFSRPGNNYI |
| 324 | 1658 | FINSTDFLY |
| 325 | 1666 | RAQQTVRNIL |
| 326 | 1668 | RNILSNDCL |
| 327 | 1669 | RTHLITTLDY |
| 328 | 1684 | NINRINIPYF |
| 329 | 1685 | RQYPGCSRVY |
| 330 | 1693 | ATQQLALNY |
| 331 | 1698 | ALSTSSTGY |
| 332 | 1701 | SSTSGVLPF |
| 333 | 1706 | SVSEPLTQY |
| 334 | 1719 | VVTPKHLTY |
| 335 | 1736 | NQNYFPVQF |
| 336 | 1744 | SFKDWIPEF |
| 337 | 1749 | SMSYFDGKTEY |
| 338 | 1750 | MSYFDGKTEY |
| 339 | 1758 | VQLANSSVYY |
| 340 | 1759 | QLANSSVYY |
| 341 | 1760 | SVYHVQEEL |
| 342 | 1761 | RAFFPCDPY |
| 343 | 1776 | TVLNTFEAY |
| 344 | 1785 | MQDGIRWFYLF |
| 345 | 1823 | SMMDFERVHY |
| 346 | 1824 | MMDFERVHY |
| 347 | 1828 | YVGKGTTIYY |
| 348 | 1835 | GKTMPVEFYY |
| 349 | 1836 | KTMPVEFYY |
| 350 | 1840 | SMFKHFDNM |
| 351 | 1849 | SLNRIVEEF |
| 352 | 1850 | GIIEFNTYY |
| 353 | 1852 | KALQGCYTY |
| 354 | 1853 | FLIDFSNLF |
| 355 | 1863 | SMPMSMIGPY |
| 356 | 1864 | SMIGPYLNVY |
| 357 | 1868 | FVQKLWAAY |
| 358 | 1872 | KLYTAALGVY |
| 359 | 1873 | YSDYVGSGY |
| 360 | 1875 | TMDPQVLNL |
| 361 | 1877 | SHLNNFLPI |
| 362 | 1880 | MTVFPFMIPF |
| 363 | 1882 | AVSDVNGMQY |
| 364 | 1896 | MVAVNLFRF |
| 365 | 1897 | YLKEVYEKY |
| 366 | 1905 | RQVHILEPY |
| 367 | 1908 | ANQKMFYSI |
| 368 | 1911 | KQFEMFNMVY |
| 369 | 1912 | KIHKKLLSPY |
| 370 | 1916 | ISFKHMTSI |
| 371 | 1923 | ILNHICHQY |
| 372 | 1929 | MDSEFFQPV |
| 373 | 1941 | YQDQQWVEV |
| 374 | 1942 | VTDNPVTDRL |
| 375 | 1944 | SAPAHPAEPY |
| 376 | 1945 | TASQTMSAI |
| 377 | 1953 | RTASSAELY |
| 378 | 1960 | SNLNNSCFI |
| 379 | 1969 | ATNFFIQPI |
| 380 | 1982 | LTHNHILFTY |
| 381 | 1986 | ATQFVQHGIY |
| 382 | 1989 | HIYETNLYL |
| 383 | 1991 | YAANLLTNY |
| 384 | 2032 | RSNTPTYLY |
| 385 | 2034 | RSNTPTYL |
| 386 | 2036 | MCGKRNCPLY |
| 387 | 2037 | CGKRNCPLYY |
| 388 | 2038 | CGKRNCPLY |
| 389 | 2044 | RNCPLYYFLL |
| 390 | 2052 | KRLPQFFLRRI |
| 391 | 2053 | KRLPQFFLRR |
| 392 | 2061 | FSNNNTFLYHF |
| 393 | 2068 | RTKFPEINI |
| 394 | 2075 | KSCYPLVF |
| 395 | 2076 | SILCSCISF |
| 396 | 2080 | KSSHNYIPL |
| 397 | 2087 | TTSANSPIVY |
| 398 | 2092 | IAFPPEYPY |
| 399 | 2097 | KIYRQVLTF |
| 400 | 2099 | MVGEYPMCY |
| 401 | 2103 | CALYFNDPF |
| 402 | 2104 | KNVSTVFTYY |
| 403 | 2118 | MQTAIQKNY |
| 404 | 2125 | TAIQKNYFRF |
| 405 | 2126 | AIQKNYFRF |
| 406 | 2127 | AIQKNYFRFFK |
| 407 | 2128 | AIQKNYFRFF |
| 408 | 2129 | IQKNYFRFFK |
| 409 | 2134 | KNYFRFFK |
| 410 | 2144 | KLLTHFNIY |
| 411 | 2146 | LLTHFNIYR |
| 412 | 2153 | QMAPGGSYF |
| 413 | 2159 | RIHTRFGQY |
| 414 | 2166 | KIDDFIRLYP |
| 415 | 2175 | RLYPHIFY |
| 416 | 2183 | PHIFYRPLY |
| 417 | 2185 | HIFYRPLYR |
| 418 | 2205 | MTSSEWIAEY |
| 419 | 2211 | SLVTVNTEY |
| 420 | 2213 | ELFSNNLLF |
| 421 | 2214 | FILDDISFSEM |
| 422 | 2218 | DDISFSEML |
| 423 | 2220 | ISFSEMLTR |
| 424 | 2221 | ISFSEMLTRYW |
| 425 | 2222 | SFSEMLTRY |
| 426 | 2223 | SFSEMLTRYWY |
| 427 | 2225 | FSEMLTRYWY |
| 428 | 2228 | SEMLTRYWYSM |
| 429 | 2229 | SEMLTRYWYS |
| 430 | 2236 | AILYNLTEAI |
| 431 | 2239 | YNLTEAIQYFY |
| 432 | 2241 | YNLTEAIQY |
| 433 | 2242 | NLTEAIQYF |
| 434 | 2245 | TEAIQYFYQRY |
| 435 | 2247 | TEAIQYFYQR |
| 436 | 2251 | AIQYFYQRYR |
| 437 | 2252 | IQYFYQRYR |
| 438 | 2253 | IQYFYQRY |
| 439 | 2255 | QYFYQRYRHF |
| 440 | 2262 | RYRHFKDWR |
| 441 | 2265 | YRHFKDWRL |
| 442 | 2266 | YRHFKDWRLI |

APPENDIX II
Positive Peptides Identified by a Full Screen,
Along with ELISpot Assay Results (Number of
Spots Counted for Each Peptide) for Animal 14S
(Swine 14 From Farm S)

| | SEQ ID | Amino Acid | Number |
|---|---|---|---|
| | NO. | Sequence | of Spots |
| 1 | 69 | KSMPLIVENSY | 255 |
| 2 | 70 | CTYAKSCDF | 35 |
| 3 | 241 | MNFFCNIKL | 16 |
| 4 | 275 | KISHYVATY | 21 |
| 5 | 278 | KTDLLNNEF | 15 |
| 6 | 279 | SLSTLLLKY | 17 |
| 7 | 280 | YAIAIRYNL | 12 |
| 8 | 283 | NVFDLHEAY | 18 |
| 9 | 285 | AMLSSIQYY | 14 |
| 10 | 297 | RHNFTKAIHY | 16 |
| 11 | 309 | TKAIHYFYK | 12 |
| 12 | 321 | KRHKNHLYWR | 14 |
| 13 | 328 | ASLDYGMNL | 13 |
| 14 | 329 | NNNTLNMFF | 12 |
| 15 | 335 | TMYSLGYIF | 12 |
| 16 | 357 | HVIQRLGLY | 13 |
| 17 | 386 | HSQIQDWHVL | 110 |
| 18 | 447 | IIHTIYQSY | 331 |
| 19 | 467 | QAYALEYAMY | 17 |
| 20 | 469 | RQYDLIQKY | 14 |
| 21 | 478 | HNITGYTYL | 33 |
| 22 | 523 | FSKPFMRFIL | 12 |
| 23 | 534 | FTFKFAAHL | 12 |
| 24 | 557 | EFLTGFFYLY | 16 |
| 25 | 585 | RAQKRELLR | 12 |
| 26 | 607 | INCFNYCILY | 12 |
| 27 | 608 | KHDARMLINY | 13 |
| 28 | 625 | GRIDFLKFLF | 15 |
| 29 | 633 | KAAIRGRSL | 14 |
| 30 | 635 | RGRSLNMLSL | 12 |
| 31 | 641 | RSLNMLSLI | 16 |
| 32 | 647 | HRGFTAWDW | 14 |
| 33 | 703 | TVITTAYNF | 13 |
| 34 | 724 | SDMYSVIFNI | 15 |
| 35 | 725 | DMYSVIFNIKY | 15 |
| 36 | 726 | DMYSVIFNI | 14 |
| 37 | 769 | AMDEAIHAALY | 13 |
| 38 | 784 | SKQASISSIL | 12 |
| 39 | 827 | QTISNHQLSF | 13 |
| 40 | 842 | SSMHSGMLYK | 13 |
| 41 | 848 | FLKKNIYLY | 19 |
| 42 | 849 | HSKALATLLY | 13 |
| 43 | 860 | RFNTLHIHY | 14 |
| 44 | 865 | RVHASYPGY | 15 |
| 45 | 869 | CTQPARVTY | 13 |
| 46 | 872 | RVDMNRFFQFY | 15 |
| 47 | 880 | KTVEPTNFL | 66 |
| 48 | 888 | MMHYPTFNW | 12 |
| 49 | 896 | ITNKIYMFF | 18 |
| 50 | 906 | WVGYNNVCY | 12 |
| 51 | 920 | SVLLRDSGYY | 17 |
| 52 | 921 | SVLLRDSGY | 14 |
| 53 | 923 | KKQKHVSLLY | 14 |
| 54 | 925 | KQKHVSLLY | 14 |
| 55 | 926 | KQKHVSLLYI | 16 |
| 56 | 931 | VSFNKTIIL | 12 |
| 57 | 954 | ISTSNETTL | 15 |
| 58 | 960 | TTLINCTYL | 13 |
| 59 | 962 | TLINCTYLTL | 12 |
| 60 | 963 | LINCTYLTL | 19 |
| 61 | 971 | LTLSSNYFYTF | 12 |
| 62 | 1006 | LLPKPYSRY | 13 |
| 63 | 1049 | AVIEAIGAM | 15 |
| 64 | 1065 | KTLKTVYPEY | 36 |
| 65 | 1090 | LFPQYISYY | 106 |
| 66 | 1106 | RTIPVAWDRF | 130 |
| 67 | 1107 | MTSLLKTDF | 18 |
| 68 | 1111 | LSYMPPNIF | 13 |
| 69 | 1120 | FSYEKNLLF | 13 |
| 70 | 1127 | RALKMYEDY | 12 |
| 71 | 1129 | ITSNVLTTF | 14 |
| 72 | 1139 | SILAEYVYSY | 12 |
| 73 | 1141 | YSYNGMLEHY | 18 |
| 74 | 1150 | NLSEVVTAY | 12 |
| 75 | 1159 | RSIETYYPEW | 12 |
| 76 | 1172 | SEDFQYWTF | 12 |
| 77 | 1187 | VSNILHSVF | 15 |
| 78 | 1188 | TSIKPVSPF | 13 |
| 79 | 1196 | KEMQDYSLTFL | 17 |
| 80 | 1204 | QDYSLTFLLK | 17 |
| 81 | 1227 | SSYNRSLLH | 12 |
| 82 | 1228 | RSSTSKSSY | 15 |
| 83 | 1264 | INRNYYPYY | 12 |
| 84 | 1265 | INRNYYPYYI | 12 |
| 85 | 1278 | YPYYIYKIF | 16 |
| 86 | 1279 | YIYKIFDAI | 12 |
| 87 | 1287 | HFNAHFKPY | 14 |
| 88 | 1288 | KPYVPVGFEY | 16 |
| 89 | 1295 | HGQLQTFPR | 17 |
| 90 | 1345 | KLMSALKWPI | 16 |
| 91 | 1347 | LMSALKWPIEY | 17 |
| 92 | 1348 | MSALKWPIEY | 12 |
| 93 | 1370 | FALKPREEY | 14 |
| 94 | 1372 | VSRAREFYI | 12 |
| 95 | 1375 | EFYISWDTDY | 15 |
| 96 | 1379 | VSASAINFL | 13 |
| 97 | 1388 | ATSHVATSY | 14 |
| 98 | 1390 | NLAGITTLM | 12 |
| 99 | 1394 | KTVPKFVPTY | 14 |
| 100 | 1400 | ESAETIYTF | 14 |
| 101 | 1413 | SSMSVSTFW | 12 |
| 102 | 1436 | KANKAFYINH | 13 |
| 103 | 1437 | KANKAFYI | 12 |
| 104 | 1454 | INHLYKFLLI | 12 |
| 105 | 1461 | LLSPLQSYTY | 13 |
| 106 | 1468 | AADDTTCYY | 13 |
| 107 | 1472 | GILSYTSLY | 16 |
| 108 | 1483 | GTSYLRMAY | 12 |
| 109 | 1484 | IAHVNTPNF | 14 |
| 110 | 1488 | HLQAFLDSY | 13 |
| 111 | 1491 | IADAINQEF | 13 |
| 112 | 1499 | FLNKSTQAY | 14 |
| 113 | 1501 | KTDPNFKNLY | 14 |
| 114 | 1503 | NQAINTFMYY | 13 |
| 115 | 1507 | RALEGLDLY | 13 |
| 116 | 1509 | FTDNAPAGHYY | 17 |
| 117 | 1510 | RSLSNFQAL | 15 |
| 118 | 1511 | QIYKTLLEY | 17 |
| 119 | 1512 | ETEDVFFTF | 21 |
| 120 | 1514 | RLAEFYQKL | 15 |
| 121 | 1517 | RTMNDFGMM | 13 |
| 122 | 1519 | MMNQTNYSI | 13 |
| 123 | 1523 | SLMADTKYF | 14 |
| 124 | 1528 | RVFSRLVFY | 13 |
| 125 | 1531 | FSQAVMEMGY | 12 |
| 126 | 1543 | GSLYPTQFDY | 12 |
| 127 | 1544 | SLYPTQFDY | 14 |
| 128 | 1556 | VVFHAGSLY | 13 |
| 129 | 1566 | QAQEEWNMIL | 14 |
| 130 | 1567 | AQEEWNMIL | 13 |
| 131 | 1571 | EQYGKAPDF | 14 |
| 132 | 1573 | IRAHNFIQTI | 12 |
| 133 | 1580 | MKQFCKISVW | 12 |
| 134 | 1619 | DFDPLVTFY | 16 |
| 135 | 1627 | SAMLEFKKF | 12 |
| 136 | 1628 | SAMLEFKKFF | 12 |
| 137 | 1630 | FTQITRQTF | 12 |
| 138 | 1631 | TQITRQTFM | 12 |
| 139 | 1633 | IADSATKEV | 14 |
| 140 | 1648 | TLMDQPTY | 14 |
| 141 | 1649 | RNLRFSRPG | 18 |
| 142 | 1651 | RFSRPGNNYI | 17 |
| 143 | 1658 | FINSTDFLY | 12 |
| 144 | 1685 | RQYPGCSRVY | 12 |
| 145 | 1693 | ATQQLALNY | 12 |
| 146 | 1701 | SSTSGVLPF | 13 |
| 147 | 1706 | SVSEPLTQY | 13 |
| 148 | 1718 | KAQFIKEGY | 15 |
| 149 | 1736 | NQNYFPVQF | 13 |
| 150 | 1749 | SMSYFDGKTEY | 15 |
| 151 | 1750 | MSYFDGKTEY | 17 |
| 152 | 1753 | FANAMQAYL | 13 |
| 153 | 1759 | QLANSSVYY | 15 |
| 154 | 1761 | RAFFPCDPY | 12 |
| 155 | 1767 | RIFAGKMLSY | 13 |
| 156 | 1783 | MQDGIRWFYL | 13 |
| 157 | 1810 | KIKNSVPSY | 13 |
| 158 | 1814 | KASPSPMEM | 17 |
| 159 | 1823 | SMMDFERVHY | 25 |
| 160 | 1824 | MMDFERVHY | 14 |
| 161 | 1828 | YVGKGTTIYY | 14 |
| 162 | 1830 | MALAKMYTL | 12 |
| 163 | 1835 | GKTMPVEFYY | 21 |
| 164 | 1836 | KTMPVEFYY | 17 |
| 165 | 1840 | SMFKHFDNM | 14 |
| 166 | 1852 | KALQGCYTY | 15 |
| 167 | 1864 | SMIGPYLNVY | 13 |
| 168 | 1873 | YSDYVGSGY | 12 |
| 169 | 1875 | TMDPQVLNL | 12 |
| 170 | 1880 | MTVFPFMIPF | 12 |
| 171 | 1912 | KIHKKLLSPY | 12 |
| 172 | 1923 | ILNHICHQY | 13 |
| 173 | 1941 | YQDQQWVEV | 12 |
| 174 | 1955 | RTARHNLSL | 14 |
| 175 | 1982 | LTHNHILFTY | 14 |
| 176 | 1986 | ATQFVQHGIY | 13 |
| 177 | 1989 | HIYETNLYL | 14 |
| 178 | 1991 | YAANLLTNY | 29 |
| 179 | 2037 | CGKRNCPLYY | 12 |
| 180 | 2038 | CGKRNCPLY | 12 |
| 181 | 2075 | KSCYPLVF | 12 |
| 182 | 2092 | IAFPPEYPY | 17 |
| 183 | 2118 | MQTAIQKNY | 13 |
| 184 | 2125 | TAIQKNYFRF | 14 |
| 185 | 2126 | AIQKNYFRF | 13 |
| 186 | 2127 | AIQKNYFRFFK | 15 |
| 187 | 2134 | KNYFRFFK | 16 |
| 188 | 2137 | NYFRFFKKL | 12 |
| 189 | 2141 | RFFKKLLTH | 12 |
| 190 | 2142 | KKLLTHFNI | 12 |
| 191 | 2146 | LLTHFNIYR | 16 |
| 192 | 2159 | RIHTRFGQY | 15 |
| 193 | 2166 | KIDDFIRLYP | 13 |
| 194 | 2175 | RLYPHIFY | 16 |
| 195 | 2181 | YPHIFYRPL | 12 |
| 196 | 2183 | PHIFYRPLY | 15 |
| 197 | 2185 | HIFYRPLYR | 15 |
| 198 | 2193 | RSCDYAPGFY | 12 |
| 199 | 2194 | TTADLQSPF | 12 |
| 200 | 2205 | MTSSEWIAEY | 13 |
| 201 | 2211 | SLVTVNTEY | 12 |
| 202 | 2213 | ELFSNNLLF | 12 |
| 203 | 2222 | SFSEMLTRY | 12 |
| 204 | 2223 | SFSEMLTRYWY | 12 |
| 205 | 2225 | FSEMLTRYWY | 12 |
| 206 | 2241 | YNLTEAIQY | 14 |
| 207 | 2242 | NLTEAIQYF | 12 |
| 208 | 2251 | AIQYFYQRYR | 12 |
| 209 | 2252 | IQYFYQRYR | 13 |
| 210 | 2265 | YRHFKDWRL | 13 |
| 211 | 2266 | YRHFKDWRLI | 14 |

APPENDIX III
Positive Peptides Identified by a Full Screen,
Along with ELISpot Assay Results (Number of
Spots Counted for Each Peptide) for Animal H
(Swine 9 From Farm H)

| | SEQ ID | Amino Acid | Number |
|---|---|---|---|
| | NO. | Sequence | of Spots |
| 1 | 56 | RYTQIYKYPL | 23 |
| 2 | 64 | VSRWYNQCTY | 32 |
| 3 | 66 | HVMDCSDPV | 62 |
| 4 | 84 | KSELSYWCTY | 16 |
| 5 | 85 | SMECLHPRPY | 20 |
| 6 | 369 | HTLQWLGLY | 16 |
| 7 | 439 | KRNVLIKGI | 16 |
| 8 | 449 | EILKLATFY | 19 |
| 9 | 458 | VSEYINYLF | 16 |
| 10 | 478 | HNITGYTYL | 19 |
| 11 | 554 | VLIEFLTGFF | 16 |
| 12 | 565 | KVMDMICLDY | 17 |
| 13 | 653 | AVFTGNMEL | 16 |
| 14 | 743 | SKFCNHMFFR | 16 |
| 15 | 744 | NHMFFRSCV | 18 |
| 16 | 756 | CFMMQCKSIY | 19 |
| 17 | 757 | FMMQCKSIY | 16 |
| 18 | 835 | RSEWASSNTF | 26 |
| 19 | 836 | SEWASSNTF | 31 |
| 20 | 839 | ASSSMHSGMLY | 16 |
| 21 | 842 | SSMHSGMLYK | 23 |
| 22 | 847 | KTFYISPNKY | 21 |
| 23 | 848 | FLKKNIYLY | 71 |
| 24 | 857 | KQMFNVDITY | 17 |
| 25 | 860 | RFNTLHIHY | 19 |
| 26 | 872 | RVDMNRFFQFY | 18 |
| 27 | 884 | SAINHFNYTM | 19 |
| 28 | 889 | ALEDHYGLY | 16 |
| 29 | 896 | ITNKIYMFF | 26 |
| 30 | 908 | VGYNNVCYYF | 16 |
| 31 | 920 | SVLLRDSGYY | 35 |
| 32 | 923 | KKQKHVSLLY | 16 |
| 33 | 977 | LSSNYFYTFF | 18 |
| 34 | 1001 | KLYYIPLSI | 21 |
| 35 | 1019 | QTNCQLYFF | 21 |
| 36 | 1020 | IITAMTHLM | 18 |
| 37 | 1024 | LVDEIYSTL | 20 |
| 38 | 1033 | FTSGYMPLLY | 16 |
| 39 | 1080 | FRDHFIALF | 16 |
| 40 | 1091 | FPQYISYY | 17 |
| 41 | 1151 | SVLEKYLQW | 16 |
| 42 | 1184 | SSIPKNKLF | 16 |
| 43 | 1187 | VSNILHSVF | 16 |
| 44 | 1205 | QDYSLTFLL | 17 |
| 45 | 1207 | DYSLTFLLK | 17 |
| 46 | 1212 | FLLKKRMEL | 19 |
| 47 | 1296 | QLQTFPRNGY | 16 |
| 48 | 1437 | KANKAFYI | 16 |
| 49 | 1454 | INHLYKFLLI | 18 |
| 50 | 1459 | SVTEFYTKL | 19 |
| 51 | 1461 | LLSPLQSYTY | 17 |
| 52 | 1767 | RIFAGKMLSY | 21 |
| 53 | 1841 | SMFKHFDNMVY | 17 |
| 54 | 1950 | LVDHIFNYL | 16 |
| 55 | 1952 | SRTASSAELY | 17 |
| 56 | 2139 | YFRFFKKLL | 19 |
| 57 | 2140 | RFFKKLLTHF | 16 |
| 58 | 2171 | IRLYPHIFYR | 18 |
| 59 | 2197 | KAIELYWVF | 17 |

APPENDIX IV
201 Positive Peptides from ELISpot Screenings

| | | Amino Acid |
|---|---|---|
| | SEQ ID NO. | Sequence |
| 1 | 1 | STNEDNQLM |
| 2 | 2 | NEMDIVQIFY |
| 3 | 3 | EMDIVQIFY |
| 4 | 8 | AMVTSVKNFY |
| 5 | 9 | MVTSVKNFY |
| 6 | 18 | KTLADIYGY |
| 7 | 26 | YIKIHQHYY |
| 8 | 32 | HQHYYINI |
| 9 | 36 | HYYINIYMYL |
| 10 | 37 | YYINIYMYL |
| 11 | 67 | YMENCKFCW |
| 12 | 69 | KSMPLIVENSY |
| 13 | 70 | CTYAKSCDF |
| 14 | 81 | YISQCSIARY |
| 15 | 84 | KSELSYWCTY |
| 16 | 87 | VLNRPLSIFY |
| 17 | 89 | YMNCSLPTYF |
| 18 | 93 | MTRNTLVLKF |
| 19 | 94 | NIDEVHHAY |
| 20 | 99 | KNTESFYPF |
| 21 | 100 | KMIKNTYVL |
| 22 | 101 | NVDEIHHAY |
| 23 | 118 | GANEKFAHY |
| 24 | 124 | FEIYFARLY |
| 25 | 128 | IYFARLYVY |
| 26 | 129 | IYFARLYVYSK |
| 27 | 159 | RKSWYWFCIF |
| 28 | 173 | YQNERYMIM |
| 29 | 180 | RAISFFYQTY |
| 30 | 185 | SMVDCCHKNY |
| 31 | 186 | LLSDNPLFL |
| 32 | 187 | TLDNISFNEM |
| 33 | 192 | ISFNEMLTRYW |
| 34 | 193 | SFNEMLTRY |
| 35 | 210 | RYWYSMAIL |
| 36 | 220 | YSMAILYK |
| 37 | 221 | MAILYKLTEAI |
| 38 | 265 | SYSAIYYCF |
| 39 | 268 | KLPEFFDEY |
| 40 | 271 | WIYENLHIY |
| 41 | 272 | HIYNMIDTF |
| 42 | 275 | KISHYVATY |
| 43 | 277 | MRIEIFWEL |
| 44 | 278 | KTDLLNNEF |
| 45 | 279 | SLSTLLLKY |
| 46 | 283 | NVFDLHEAY |
| 47 | 284 | QAMLSSIQYY |
| 48 | 285 | AMLSSIQYY |
| 49 | 294 | ALRHNFTKAI |
| 50 | 302 | HNFTKAIHY |
| 51 | 308 | FTKAIHYFYKR |
| 52 | 312 | KAIHYFYK |
| 53 | 313 | KAIHYFYKRHK |
| 54 | 343 | STCSLKCLF |
| 55 | 357 | HVIQRLGLY |
| 56 | 360 | KKTLNLLLSY |
| 57 | 363 | YMVDFMREF |
| 58 | 364 | RLHYLKSLVY |
| 59 | 365 | KEMFNLARFY |
| 60 | 369 | HTLQWLGLY |
| 61 | 370 | QIQDWHILL |
| 62 | 371 | MAIDNGLLPF |
| 63 | 375 | KQIVHTIKY |
| 64 | 377 | NTFFLPSDF |
| 65 | 386 | HSQIQDWHVL |
| 66 | 394 | NMLSILVKY |
| 67 | 400 | RKLEILTWM |
| 68 | 404 | EMFSLGYKI |
| 69 | 405 | SLGYKIVFEY |
| 70 | 435 | STYFQVKEF |
| 71 | 447 | IIHTIYQSY |
| 72 | 449 | EILKLATFY |
| 73 | 452 | RRTESKKLFL |
| 74 | 455 | SIVSEYINY |
| 75 | 456 | IVSEYINYL |
| 76 | 457 | IVSEYINYLF |
| 77 | 461 | EIMQAYALEY |
| 78 | 462 | IMQAYALEY |
| 79 | 463 | MQAYALEYAM |
| 80 | 467 | QAYALEYAMY |
| 81 | 468 | HVVQRLGLY |
| 82 | 469 | RQYDLIQKY |
| 83 | 478 | HNITGYTYL |
| 84 | 486 | AAAGGLLNF |
| 85 | 492 | IVDDYIRFLF |
| 86 | 496 | ISLRLFEVK |
| 87 | 497 | RLFEVKPKY |
| 88 | 527 | LANAFIPPY |
| 89 | 529 | RKYIHKIIL |
| 90 | 541 | CNACKTLNY |
| 91 | 544 | LNYKHYKTL |
| 92 | 547 | SHLEGFMRTY |
| 93 | 548 | EGFMRTYLL |
| 94 | 549 | RYIWSGLVY |
| 95 | 553 | VLIEFLTGF |
| 96 | 554 | VLIEFLTGFF |
| 97 | 559 | TGFFYLYGK |
| 98 | 561 | KRLFSISKVM |
| 99 | 570 | YTIIPAPLAM |
| 100 | 578 | KNYDLMKRL |
| 101 | 584 | VVDDVPSIDY |
| 102 | 588 | KYLMNCSGF |
| 103 | 589 | KNIIKELVF |
| 104 | 619 | HIRNGNLTLF |
| 105 | 621 | KTDPWIVNR |
| 106 | 633 | KAAIRGRSL |
| 107 | 636 | RGRSLNMLSLI |
| 108 | 639 | GRSLNMLSL |
| 109 | 640 | GRSLNMLSLI |
| 110 | 645 | SLNMLSLIKF |
| 111 | 647 | HRGFTAWDW |
| 112 | 651 | FTAWDWAVF |
| 113 | 652 | WAVFTGNMEL |
| 114 | 653 | AVFTGNMEL |
| 115 | 662 | LRKSPKLLL |
| 116 | 670 | YTLCDSPAY |
| 117 | 680 | SIIPFADAL |
| 118 | 681 | RTVEICCRY |
| 119 | 711 | FLAFSLHSDMY |
| 120 | 713 | LAFSLHSDMY |
| 121 | 728 | MYSVIFNIK |
| 122 | 731 | YSVIFNIKYF |
| 123 | 732 | SVIFNIKYF |
| 124 | 743 | SKFCNHMFFR |
| 125 | 773 | VSQSMSLNY |
| 126 | 796 | ISSILNFFF |
| 127 | 822 | YFAPSFAIF |
| 128 | 828 | ISNHQLSFTY |
| 129 | 885 | AINHFNYTM |
| 130 | 888 | MMHYPTFNW |
| 131 | 914 | KTLNLTKTY |
| 132 | 927 | VSLLYICSK |
| 133 | 957 | NETTLINCTY |
| 134 | 1012 | RYQYNTPIYY |
| 135 | 1019 | QTNCQLYFF |
| 136 | 1049 | AVIEAIGAM |
| 137 | 1064 | KTRGTRLFF |
| 138 | 1069 | YLMQHFRDH |
| 139 | 1096 | QYISYYTKY |
| 140 | 1104 | SISYIPLIY |
| 141 | 1106 | RTIPVAWDRF |
| 142 | 1111 | LSYMPPNIF |
| 143 | 1141 | YSYNGMLEHY |
| 144 | 1156 | YGVETHWPLY |
| 145 | 1188 | TSIKPVSPF |
| 146 | 1196 | KEMQDYSLTFL |
| 147 | 1203 | MQDYSLTF |
| 148 | 1233 | RQTMMSSIY |
| 149 | 1248 | KTLISEMMHY |
| 150 | 1253 | RTDLNNCVSL |
| 151 | 1256 | KGRINRNYY |
| 152 | 1280 | TTNRRILQY |
| 153 | 1282 | ASGGAFCLI |
| 154 | 1288 | KPYVPVGFEY |
| 155 | 1295 | HGQLQTFPR |
| 156 | 1325 | VSIPFGERF |
| 157 | 1340 | VTPEIHNLF |
| 158 | 1345 | KLMSALKWPI |
| 159 | 1348 | MSALKWPIEY |
| 160 | 1372 | VSRAREFYI |
| 161 | 1374 | RAREFYISW |
| 162 | 1380 | VSASAINFLL |
| 163 | 1437 | KANKAFYI |
| 164 | 1440 | ANKAFYINHL |
| 165 | 1464 | LTQRPVMGY |
| 166 | 1472 | GILSYTSLY |
| 167 | 1512 | ETEDVFFTF |
| 168 | 1531 | FSQAVMEMGY |
| 169 | 1543 | GSLYPTQFDY |
| 170 | 1556 | VVFHAGSLY |
| 171 | 1560 | SGRIVTTAI |
| 172 | 1561 | VTTAIKTLL |
| 173 | 1584 | MLGNLSAAKY |
| 174 | 1623 | FLIPETILF |
| 175 | 1635 | YSDPETVHSY |
| 176 | 1652 | FSRPGNNYI |
| 177 | 1653 | ELNITSPAM |
| 178 | 1676 | VVIMAIMLY |
| 179 | 1715 | RVYLDGELY |
| 180 | 1740 | ASSIVSNLF |
| 181 | 1744 | SFKDWIPEF |
| 182 | 1745 | FQNFSKSLY |
| 183 | 1823 | SMMDFERVHY |
| 184 | 1832 | KTHYSIPSSF |
| 185 | 1836 | KTMPVEFYY |
| 186 | 1860 | AAFNQQYIF |
| 187 | 1865 | FMNFDPAHNEY |
| 188 | 1911 | KQFEMFNMVY |
| 189 | 1924 | GVKHFLHEY |
| 190 | 1929 | MDSEFFQPV |
| 191 | 1952 | SRTASSAELY |
| 192 | 1991 | YAANLLTNY |
| 193 | 2020 | KTFQDIRII |
| 194 | 2021 | CGMKNISEI |
| 195 | 2044 | RNCPLYYFLL |
| 196 | 2049 | YLKRLPQFF |
| 197 | 2112 | IISMMQTAI |
| 198 | 2113 | ISMMQTAIQK |
| 199 | 2136 | KNYFRFFKKL |
| 200 | 2204 | SVEELLSAV |
| 201 | 2205 | MTSSEWIAEY |

APPENDIX V
77 Positive Peptides from ELISpot Screenings,
and Corresponding ASFV Proteins

| | SEQ | Amino Acid | Corresponding |
|---|---|---|---|
| | ID NO. | Sequence | ASFV Protein |
| 1 | 32 | HQHYYINI | AYW33961.1 |
| 2 | 67 | YMENCKFCW | AYW33963.1 |
| 3 | 69 | KSMPLIVENSY | AYW33963.1 |
| 4 | 70 | CTYAKSCDF | AYW33964.1 |
| 5 | 101 | NVDEIHHAY | AYW33969.1 |
| 6 | 128 | IYFARLYVY | AYW33971.1 |
| 7 | 187 | TLDNISFNEM | AYW33974.1 |
| 8 | 278 | KTDLLNNEF | AYW33992.1 |
| 9 | 279 | SLSTLLLKY | AYW33992.1 |
| 10 | 363 | YMVDFMREF | AYW33999.1 |
| 11 | 377 | NTFFLPSDF | AYW34001.1 |
| 12 | 400 | RKLEILTWM | AYW34001.1 |
| 13 | 404 | EMFSLGYKI | AYW34001.1 |
| 14 | 435 | STYFQVKEF | AYW34001.1 |
| 15 | 447 | IIHTIYQSY | AYW34001.1 |
| 16 | 449 | EILKLATFY | AYW34001.1 |
| 17 | 455 | SIVSEYINY | AYW34001.1 |
| 18 | 456 | IVSEYINYL | AYW34001.1 |
| 19 | 457 | IVSEYINYLF | AYW34001.1 |
| 20 | 461 | EIMQAYALEY | AYW34001.1 |
| 21 | 462 | IMQAYALEY | AYW34001.1 |
| 22 | 463 | MQAYALEYAM | AYW34001.1 |
| 23 | 467 | QAYALEYAMY | AYW34001.1 |
| 24 | 468 | HVVQRLGLY | AYW34002.1 |
| 25 | 469 | RQYDLIQKY | AYW34002.1 |
| 26 | 478 | HNITGYTYL | AYW34002.1 |
| 27 | 486 | AAAGGLLNF | AYW34003.1 |
| 28 | 492 | IVDDYIRFLF | AYW34003.1 |
| 29 | 496 | ISLRLFEVK | AYW34004.1 |
| 30 | 497 | RLFEVKPKY | AYW34004.1 |
| 31 | 527 | LANAFIPPY | AYW34004.1 |
| 32 | 529 | RKYIHKIIL | AYW34004.1 |
| 33 | 541 | CNACKTLNY | AYW34004.1 |
| 34 | 544 | LNYKHYKTL | AYW34004.1 |
| 35 | 547 | SHLEGFMRTY | AYW34005.1 |
| 36 | 548 | EGFMRTYLL | AYW34005.1 |
| 37 | 549 | RYIWSGLVY | AYW34007.1 |
| 38 | 553 | VLIEFLTGF | AYW34010.1 |
| 39 | 554 | VLIEFLTGFF | AYW34010.1 |
| 40 | 561 | KRLFSISKVM | AYW34010.1 |
| 41 | 578 | KNYDLMKRL | AYW34010.1 |
| 42 | 584 | VVDDVPSIDY | AYW34010.1 |
| 43 | 589 | KNIIKELVF | AYW34010.1 |
| 44 | 619 | HIRNGNLTLF | AYW34011.1 |
| 45 | 621 | KTDPWIVNR | AYW34011.1 |
| 46 | 633 | KAAIRGRSL | AYW34011.1 |
| 47 | 636 | RGRSLNMLSLI | AYW34011.1 |
| 48 | 639 | GRSLNMLSL | AYW34011.1 |
| 49 | 640 | GRSLNMLSLI | AYW34011.1 |
| 50 | 645 | SLNMLSLIKF | AYW34011.1 |
| 51 | 651 | FTAWDWAVF | AYW34011.1 |
| 52 | 652 | WAVFTGNMEL | AYW34011.1 |
| 53 | 653 | AVFTGNMEL | AYW34011.1 |
| 54 | 662 | LRKSPKLLL | AYW34011.1 |
| 55 | 670 | YTLCDSPAY | AYW34012.1 |
| 56 | 711 | FLAFSLHSDMY | AYW34013.1 |
| 57 | 713 | LAFSLHSDMY | AYW34013.1 |
| 58 | 743 | SKFCNHMFFR | AYW34013.1 |
| 59 | 1049 | AVIEAIGAM | AYW34032.1 |
| 60 | 1106 | RTIPVAWDRF | AYW34037.1 |
| 61 | 1156 | YGVETHWPLY | AYW34044.1 |
| 62 | 1248 | KTLISEMMHY | AYW34052.1 |
| 63 | 1253 | RTDLNNCVSL | AYW34052.1 |
| 64 | 1280 | TTNRRILQY | AYW34052.1 |
| 65 | 1282 | ASGGAFCLI | AYW34053.1 |
| 66 | 1288 | KPYVPVGFEY | AYW34053.1 |
| 67 | 1437 | KANKAFYI | AYW34060.1 |
| 68 | 1440 | ANKAFYINHL | AYW34060.1 |
| 69 | 1531 | FSQAVMEMGY | AYW34063.1 |
| 70 | 1556 | VVFHAGSLY | AYW34064.1 |
| 71 | 1560 | SGRIVTTAI | AYW34064.1 |
| 72 | 1561 | VTTAIKTLL | AYW34064.1 |
| 73 | 1584 | MLGNLSAAKY | AYW34065.1 |
| 74 | 1991 | YAANLLTNY | AYW34108.1 |
| 75 | 2021 | CGMKNISEI | AYW34111.1 |
| 76 | 2112 | IISMMQTAI | AYW34117.1 |
| 77 | 2204 | SVEELLSAV | AYW34126.1 |

APPENDIX VI
18 “Top” Peptides from ELISpot Screenings,
and Corresponding ASFV Proteins

| | SEQ | Amino Acid | Corresponding |
|---|---|---|---|
| | ID NO. | Sequence | ASFV Protein |
| 1 | 67 | YMENCKFCW | AYW33963.1 |
| 2 | 69 | KSMPLIVENSY | AYW33963.1 |
| 3 | 70 | CTYAKSCDF | AYW33964.1 |
| 4 | 279 | SLSTLLLKY | AYW33992.1 |
| 5 | 435 | STYFQVKEF | AYW34001.1 |
| 6 | 461 | EIMQAYALEY | AYW34001.1 |
| 7 | 469 | RQYDLIQKY | AYW34002.1 |
| 8 | 478 | HNITGYTYL | AYW34002.1 |
| 9 | 486 | AAAGGLLNF | AYW34003.1 |
| 10 | 547 | SHLEGFMRTY | AYW34005.1 |
| 11 | 548 | EGFMRTYLL | AYW34005.1 |
| 12 | 549 | RYIWSGLVY | AYW34007.1 |
| 13 | 561 | KRLFSISKVM | AYW34010.1 |
| 14 | 589 | KNIIKELVF | AYW34010.1 |
| 15 | 639 | GRSLNMLSL | AYW34011.1 |
| 16 | 652 | WAVFTGNMEL | AYW34011.1 |
| 17 | 653 | AVFTGNMEL | AYW34011.1 |
| 18 | 1253 | RTDLNNCVSL | AYW34052.1 |

APPENDIX VII
Forty-four Peptides of Appendices V
and/or VI Clustered Within Seven
ASFV Proteins

| | | | Peptide |
|---|---|---|---|
| | | Peptide | Amino |
| | ASFV | SEQ ID | Acid |
| | Protein | NO. | Sequence |
| 1 | AYW34011.1 | 619 | HIRNGNLTLF |
| | | 621 | KTDPWIVNR |
| | | 633 | KAAIRGRSL |
| | | 636 | RGRSLNMLSLI |
| | | 639 | GRSLNMLSL |
| | | 640 | GRSLNMLSLI |
| | | 645 | SLNMLSLIKF |
| | | 651 | FTAWDWAVF |
| | | 652 | WAVFTGNMEL |
| | | 653 | AVFTGNMEL |
| | | 662 | LRKSPKLLL |
| 2 | AYW34004.1 | 496 | ISLRLFEVK |
| | | 497 | RLFEVKPKY |
| | | 527 | LANAFIPPY |
| | | 529 | RKYIHKIIL |
| | | 541 | CNACKTLNY |
| | | 544 | LNYKHYKTL |
| 3 | AYW34001.1 | 377 | NTFFLPSDF |
| | | 400 | RKLEILTWM |
| | | 404 | EMFSLGYKI |
| | | 435 | STYFQVKEF |
| | | 447 | IIHTIYQSY |
| | | 449 | EILKLATFY |
| | | 455 | SIVSEYINY |
| | | 456 | IVSEYINYL |
| | | 457 | IVSEYINYLF |
| | | 461 | EIMQAYALEY |
| | | 462 | IMQAYALEY |
| | | 463 | MQAYALEYAM |
| | | 467 | QAYALEYAMY |
| 4 | AYW34010.1 | 553 | VLIEFLTGF |
| | | 554 | VLIEFLTGFF |
| | | 561 | KRLFSISKVM |
| | | 578 | KNYDLMKRL |
| | | 584 | VVDDVPSIDY |
| | | 589 | KNIIKELVF |
| 5 | AYW34052.1 | 1248 | KTLISEMMHY |
| | | 1253 | RTDLNNCVSL |
| | | 1280 | TTNRRILQY |
| 6 | AYW34002.1 | 468 | HVVQRLGLY |
| | | 469 | RQYDLIQKY |
| | | 478 | HNITGYTYL |
| 7 | AYW33963.1 | 67 | YMENCKFCW |
| | | 69 | KSMPLIVENSY |

APPENDIX VIII
Peptides (125 total) IV that of APPENDIX
Met or Exceeded the Stringent
Threshold

| | SEQ | |
|---|---|---|
| | ID | Amino Acid |
| | NO. | Sequence |
| 1 | 1 | STNEDNQLM |
| 2 | 2 | NEMDIVQIFY |
| 3 | 3 | EMDIVQIFY |
| 4 | 8 | AMVTSVKNFY |
| 5 | 9 | MVTSVKNFY |
| 6 | 18 | KTLADIYGY |
| 7 | 26 | YIKIHQHYY |
| 8 | 36 | HYYINIYMYL |
| 9 | 67 | YMENCKFCW |
| 10 | 69 | KSMPLIVENSY |
| 11 | 70 | CTYAKSCDF |
| 12 | 81 | YISQCSIARY |
| 13 | 84 | KSELSYWCTY |
| 14 | 87 | VLNRPLSIFY |
| 15 | 89 | YMNCSLPTYF |
| 16 | 93 | MTRNTLVLKF |
| 17 | 101 | NVDEIHHAY |
| 18 | 124 | FEIYFARLY |
| 19 | 128 | IYFARLYVY |
| 20 | 129 | IYFARLYVYSK |
| 21 | 159 | RKSWYWFCIF |
| 22 | 185 | SMVDCCHKNY |
| 23 | 186 | LLSDNPLFL |
| 24 | 187 | TLDNISFNEM |
| 25 | 210 | RYWYSMAIL |
| 26 | 221 | MAILYKLTEAI |
| 27 | 268 | KLPEFFDEY |
| 28 | 272 | HIYNMIDTF |
| 29 | 275 | KISHYVATY |
| 30 | 278 | KTDLLNNEF |
| 31 | 279 | SLSTLLLKY |
| 32 | 285 | AMLSSIQYY |
| 33 | 294 | ALRHNFTKAI |
| 34 | 357 | HVIQRLGLY |
| 35 | 360 | KKTLNLLLSY |
| 36 | 363 | YMVDFMREF |
| 37 | 365 | KEMFNLARFY |
| 38 | 369 | HTLQWLGLY |
| 39 | 371 | MAIDNGLLPF |
| 40 | 394 | NMLSILVKY |
| 41 | 400 | RKLEILTWM |
| 42 | 404 | EMFSLGYKI |
| 43 | 435 | STYFQVKEF |
| 44 | 447 | IIHTIYQSY |
| 45 | 449 | EILKLATFY |
| 46 | 456 | IVSEYINYL |
| 47 | 457 | IVSEYINYLF |
| 48 | 461 | EIMQAYALEY |
| 49 | 462 | IMQAYALEY |
| 50 | 463 | MQAYALEYAM |
| 51 | 467 | QAYALEYAMY |
| 52 | 468 | HVVQRLGLY |
| 53 | 469 | RQYDLIQKY |
| 54 | 478 | HNITGYTYL |
| 55 | 486 | AAAGGLLNF |
| 56 | 492 | IVDDYIRFLF |
| 57 | 496 | ISLRLFEVK |
| 58 | 497 | RLFEVKPKY |
| 59 | 527 | LANAFIPPY |
| 60 | 541 | CNACKTLNY |
| 61 | 544 | LNYKHYKTL |
| 62 | 547 | SHLEGFMRTY |
| 63 | 548 | EGFMRTYLL |
| 64 | 549 | RYIWSGLVY |
| 65 | 553 | VLIEFLTGF |
| 66 | 554 | VLIEFLTGFF |
| 67 | 561 | KRLFSISKVM |
| 68 | 570 | YTIIPAPLAM |
| 69 | 578 | KNYDLMKRL |
| 70 | 588 | KYLMNCSGF |
| 71 | 589 | KNIIKELVF |
| 72 | 619 | HIRNGNLTLF |
| 73 | 621 | KTDPWIVNR |
| 74 | 633 | KAAIRGRSL |
| 75 | 639 | GRSLNMLSL |
| 76 | 640 | GRSLNMLSLI |
| 77 | 645 | SLNMLSLIKF |
| 78 | 651 | FTAWDWAVF |
| 79 | 652 | WAVFTGNMEL |
| 80 | 653 | AVFTGNMEL |
| 81 | 662 | LRKSPKLLL |
| 82 | 711 | FLAFSLHSDMY |
| 83 | 713 | LAFSLHSDMY |
| 84 | 728 | MYSVIFNIK |
| 85 | 743 | SKFCNHMFFR |
| 86 | 773 | VSQSMSLNY |
| 87 | 885 | AINHFNYTM |
| 88 | 888 | MMHYPTFNW |
| 89 | 957 | NETTLINCTY |
| 90 | 1012 | RYQYNTPIYY |
| 91 | 1049 | AVIEAIGAM |
| 92 | 1064 | KTRGTRLFF |
| 93 | 1069 | YLMQHFRDH |
| 94 | 1106 | RTIPVAWDRF |
| 95 | 1111 | LSYMPPNIF |
| 96 | 1233 | RQTMMSSIY |
| 97 | 1248 | KTLISEMMHY |
| 98 | 1253 | RTDLNNCVSL |
| 99 | 1256 | KGRINRNYY |
| 100 | 1280 | TTNRRILQY |
| 101 | 1282 | ASGGAFCLI |
| 102 | 1288 | KPYVPVGFEY |
| 103 | 1295 | HGQLQTFPR |
| 104 | 1325 | VSIPFGERF |
| 105 | 1345 | KLMSALKWPI |
| 106 | 1348 | MSALKWPIEY |
| 107 | 1437 | KANKAFYI |
| 108 | 1440 | ANKAFYINHL |
| 109 | 1531 | FSQAVMEMGY |
| 110 | 1556 | VVFHAGSLY |
| 111 | 1560 | SGRIVTTAI |
| 112 | 1561 | VTTAIKTLL |
| 113 | 1715 | RVYLDGELY |
| 114 | 1860 | AAFNQQYIF |
| 115 | 1865 | FMNFDPAHNEY |
| 116 | 1911 | KQFEMFNMVY |
| 117 | 1929 | MDSEFFQPV |
| 118 | 1952 | SRTASSAELY |
| 119 | 1991 | YAANLLTNY |
| 120 | 2021 | CGMKNISEI |
| 121 | 2049 | YLKRLPQFF |
| 122 | 2112 | IISMMQTAI |
| 123 | 2113 | ISMMQTAIQK |
| 124 | 2136 | KNYFRFFKKL |
| 125 | 2204 | SVEELLSAV |

We claim:

1. A peptide, comprising at least a portion of one or more amino acid sequences selected from SEQ ID NOs: 2310-2322 or 2330-2335.

2. The peptide of claim 1, further comprising one or more spacer sequences located between two or more of the amino acid sequences.

3. The peptide of claim 2, wherein the one or more spacer sequences comprise GPGPG, AAY, or a combination thereof.

4. An isolated nucleic acid molecule encoding an amino acid sequence having at least 85% sequence identity to the peptide of claim 1.

5. An isolated nucleic acid molecule encoding an amino acid sequence the amino acid sequence of the peptide of claim 1.

6. The isolated nucleic acid molecule of claim 4, operably linked to an expression control sequence, a selection-related sequence, a sequence comprising multiple cloning sites, or a combination thereof.

7. A viral vector comprising the isolated nucleic acid molecule of claim 4, wherein the vector is a viral vector.

8. The viral vector of claim 7, wherein the virus is a Herpesvirus, Adenovirus, Circovirus, Alphavirus, Orthopoxvirus, Avulavirus, or Poxvirus.

9. The viral vector of claim 8, wherein the virus is a Pseudorabies virus, Porcine circovirus, Sindbis virus, Vaccinia virus, Newcastle virus, or Suipoxvirus.

10. An isolated host cell comprising the vector of claim 7.

11. The isolated host cell of claim 10, wherein the cell is a recombinant yeast cell or a recombinant bacterial cell.

12. The isolated host cell of claim 10, wherein the cell is a recombinant yeast cell selected from the genus Saccharomyces or Pichia.

13. The isolated host cell of claim 10, wherein the cell is a recombinant bacterial cell selected from the genus Salmonella, Escherichia, Listeria, Shigella, Pseudomonas, Bordetella, Bacillus, Yersinia, Mycobacterium, Lactobacillus, Lactococcus, or Vibrio.

14. A composition, comprising:
the peptide of claim 1 or a nucleic acid encoding the peptide of claim 1; and
an adjuvant.

15. A composition, comprising:
the vector of claim 7; and
an adjuvant.

16. A composition, comprising:
at least one peptide, or a nucleic acid encoding the at least one peptide, wherein the at least one peptide comprises an amino acid sequence having at least 85% sequence identity to 5 or more consecutive amino acids of an amino acid sequence encoded by SEQ ID NO: 1; and
an adjuvant.

17. The composition according to claim 16 comprising at least one peptide comprising an amino acid sequence having at least 85% sequence identity to 5 or more consecutive amino acids of an amino acid sequence encoded by any one of SEQ ID NOs: 2339-2345.

18. The composition of claim 16, wherein the at least one peptide comprises an amino acid sequence having at least 85% sequence identity to an amino acid sequence encoded by any one of SEQ ID NOs: 2274-2291.

19. The composition of claim 18, wherein the at least one peptide comprises at least one construct sequence, and the sequence has at least 85% sequence identity to any one of SEQ ID NOs: 2310-2322 or 2330-2335.

20. The composition of claim 16, wherein the at least one peptide is 5 to 50 amino acids in length.

21. The composition of claim 16, comprising 2 or more peptides.

22. The composition of claim 21, further comprising one or more spacer sequences located between at least two peptides.

23. The composition of claim 16, wherein the at least one adjuvant is selected from oil adjuvants, oil-in-water adjuvants, water-in-oil adjuvants, water-in-oil-in-water adjuvants, immune-stimulating complexes (ISCOMs), liposomes, polysaccharides, derivatized polysaccharides, oligonucleotides, cytokines, bacterial derivatives, viral derivatives, aluminum hydroxide, potassium hydroxide, complete Freund's adjuvant, incomplete Freund's adjuvant, saponin, squalene, gel adjuvants, polyacrylic acid adjuvants, or a combination thereof.

24. The composition of claim 16, wherein the at least one adjuvant is Carbigen 222.

25. The composition of claim 16, further comprising at least one additional component selected from a carrier, at least one additional therapeutic, or a combination thereof.

26. The composition of claim 16, wherein the at least one peptide is glycosylated, PEGylated, lipidated, cyclized, acetylated, amidated, conjugated, has undergone D-amino acid incorporation, or a combination thereof.

27. The composition of claim 16, formulated for administration to an animal by injection, aerosol delivery, mucosal administration, oral administration, topical administration, or a combination thereof.

28. The composition of claim 16, wherein the composition reduces or prevents infection by ASFV in an animal, reduces or ameliorates at least one symptom associated with ASFV in an animal, prevents mortality associated with ASFV in an animal, or a combination thereof, relative to an animal that is not administered the composition.

29. The composition of claim 28, wherein the animal is a swine.

30. A vaccine, comprising at least one peptide of claim 1.

31. A vaccine, comprising the composition of claim 16.

32. A method, comprising administering to swine an effective amount of the vaccine according to claim 30.

33. A method, comprising administering to swine an effective amount of the vaccine according to claim 31.
